SEQUENCE LISTING (36 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 537 bp, Artificial Sequence
  Description of Artificial Sequence Synthetic polynucleotide
atgcactctagcgccctgctgtgttgtctggtgctgctgacaggcgtcagagcctctcctggacagggaacccagagcgagaatagctgcacccactttccaggcaacctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactaa

SEQ ID NO: 3 - protein, 181 aa, Homo sapiens
MGWRAAGALLLALLLHGRLLASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - DNA, 546 bp, Artificial Sequence
  Description of Artificial Sequence Synthetic polynucleotide
atgggctggcgcgccgccggcgccctgctgctggccctgctgctgcacggccgcctgctggccagccccggccagggcacccagagcgagaacagctgcacccacttccccggcaacctgcccaacatgctgcgcgacctgcgcgacgccttcagccgcgtgaagaccttcttccagatgaaggaccagctggacaacctgctgctgaaggagagcctgctggaggacttcaagggctacctgggctgccaggccctgagcgagatgatccagttctacctggaggaggtgatgccccaggccgagaaccaggaccccgacatcaaggcccacgtgaacagcctgggcgagaacctgaagaccctgcgcctgcgcctgcgccgctgccaccgcttcctgccctgcgagaacaagagcaaggccgtggagcaggtgaagaacgccttcaacaagctgcaggagaagggcatctacaaggccatgagcgagttcgacatcttcatcaactacatcgaggcctacatgaccatgaagatccgcaactaa

SEQ ID NO: 5 - DNA, 546 bp, Artificial Sequence
  Description of Artificial Sequence Synthetic polynucleotide
atgggctggcgagctgctggtgcacttctgctggctctgctgcttcatggcagactgctggcttctcctggacagggcacacagagcgagaacagctgcacacacttccccggcaacctgcctaacatgctgcgggatctgagagatgccttcagccgcgtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgaacagcctgggcgagaacctgaaaaccctgcggctgagactgcggcggtgccacagatttctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaactaa

SEQ ID NO: 6 - protein, 178 aa, Artificial Sequence
  Description of Artificial Sequence Synthetic polypeptide
MHSSALLCCLVLLTGVRASPGQGTQSENSCTHFPGILPNMLRDLRDAFSRVKTFFQMKDQLDNLLLKESLLEDFKGYLGCQALSEMIQFYLEEVMPQAENQDPDIKAHVISLGENLNTLRLRLRRCHRLLPCENKSKAVEQVKNAFNKLQEKGIYKAMSEFDIFINYIEAYMTMKIRN

SEQ ID NO: 7 - DNA, 534 bp, Artificial Sequence
  Description of Artificial Sequence Synthetic polynucleotide
atgcactctagcgccctgctgtgttgtctggtgctgctgacaggcgtcagagcctctcctggacagggaacccagagcgagaatagctgcacccactttccaggcatcctgcctaacatgctgcgggacctgagagatgccttcagcagagtgaaaacattcttccagatgaaggaccagctggacaacctgctgctgaaagagagcctgctggaagatttcaagggctacctgggctgtcaggccctgagcgagatgatccagttctacctggaagaagtgatgccccaggccgagaatcaggaccccgatattaaggcccacgtgatcagcctgggcgagaacctgaataccctgcggctgagactgcggagatgccacagactgctgccctgcgagaacaagagcaaggccgtggaacaagtgaagaacgccttcaacaagctgcaagagaagggcatctacaaggccatgtccgagttcgacatcttcatcaactacatcgaggcctacatgaccatgaagatccggaac

SEQ ID NO: 8 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 9 - DNA, 540 bp, Homo sapiens
atggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttga

SEQ ID NO: 10 - DNA, 540 bp, Artificial Sequence
  Description of Artificial Sequence Synthetic polynucleotide
atggccgctctgcagaaaagcgtgtccagcttcctgatgggcaccctggccacatcttgtctgctgctgcttgccctgctggtgcaaggcggagctgctgcccctatcagctctcactgcagactggacaagagcaacttccagcagccttacatcaccaaccggaccttcatgctggccaaagaagccagcctggccgacaacaataccgacgtgcggctgatcggcgagaagctgtttcatggcgtgtccatgagcgagcggtgctacctgatgaagcaggtcctgaacttcaccctggaagaggtgctgttccctcagagcgaccggtttcagccctacatgcaagaggtggtgcccttcctggccagactgagcaacagactgtccacctgtcacatcgagggcgacgacctgcacatccagagaaacgtgcagaaactgaaggacaccgtgaagaagctgggcgagagcggagagatcaaggccatcggagaactggacctgctgttcatgagcctgcggaacgcctgtatctaa

SEQ ID NO: 11 - protein, 167 aa, Homo sapiens
MGWRAAGALLLALLLHGRLLAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 12 - DNA, 504 bp, Artificial Sequence
  Description of Artificial Sequence Synthetic polynucleotide
atgggctggcgagctgctggtgcacttctgctggctctgctgcttcatggcagactgctggccgctcctatcagcagccactgcagactggacaagagcaacttccagcagccttacatcaccaaccggaccttcatgctggccaaagaagccagcctggccgacaacaataccgacgtgcggctgatcggcgagaagctgtttcatggcgtgtccatgagcgagcggtgctacctgatgaagcaggtcctgaacttcaccctggaagaggtgctgttccctcagagcgaccggtttcagccctacatgcaagaggtggtgcccttcctggccagactgagcaacagactgtccacctgtcacatcgagggcgacgacctgcacatccagagaaacgtgcagaaactgaaggacaccgtgaagaagctgggcgagagcggagagatcaaggccatcggagaactggacctgctgttcatgagcctgcggaacgcctgtatctaa

SEQ ID NO: 13 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 14 - protein, 378 aa, Artificial Sequence
  Description of Artificial Sequence Synthetic polypeptide
MGWRAAGALLLALLLHGRLLAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1137 bp, Artificial Sequence
  Description of Artificial Sequence Synthetic polynucleotide
atgggctggcgagctgctggtgcacttctgctggctctgctgcttcatggcagactgctggccatgcatgtggctcagcctgctgtggtgctggcctcttctagaggaatcgccagcttcgtgtgcgagtacgcctctcctggaaaggccactgaagtgcgcgtgaccgttctgagacaggccgatagccaagtgaccgaagtgtgcgccgccacctacatgatgggcaacgagctgaccttcctggacgacagcatctgtaccggcaccagctccggcaatcaagtgaacctgaccatccagggcctgagagccatggataccggcctgtacatctgcaaggtggaactgatgtaccctcctccttactacctcggcatcggcaacggcacccagatctacgtgatcgaccctgagccttgtcctgacagcgaccaagagcctaagagcagcgacaagacccacacaagccctccatctccagctccagaactgctcggcggcagctccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggatgagctgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgtctctgagccctggcaaataa

SEQ ID NO: 16 - protein, 378 aa, Artificial Sequence
  Description of Artificial Sequence Synthetic polypeptide
MGWRAAGALLLALLLHGRLLAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1137 bp, Artificial Sequence
  Description of Artificial Sequence Synthetic polynucleotide
atgggctggcgagctgctggtgcacttctgctggctctgctgcttcatggcagactgctggccatgcatgtggctcagcctgctgtggtgctggcctcttctagaggaatcgccagcttcgtgtgcgagtacgcctctcctggcaagtacaccgaagtgcgcgtgaccgttctgagacaggccgatagccaagtgaccgaagtgtgcgccgccacctacatgatgggcaacgagctgaccttcctggacgacagcatctgtaccggcaccagcagcggcaatcaagtgaacctgaccatccagggcctgagagccatggataccggcctgtacatctgcaaggtggaactgatgtaccctccgccttactacgaaggcatcggcaacggcacccagatctacgtgatcgaccctgagccttgtcctgacagcgaccaagagcctaagagcagcgacaagacccacacaagccctccatctccagctccagaactgctcggcggcagctccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggatgagctgaccaagaatcaggtgtccctgacctgcctggtcaagggcttctacccttccgatatcgccgtggaatgggagagcaatggccagcctgagaacaactacaagaccacacctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgtctctgagccctggcaaataa

SEQ ID NO: 18 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 19 - protein, 429 aa, Artificial Sequence
  Description of Artificial Sequence Synthetic polypeptide
MGWRAAGALLLALLLHGRLLAMRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEETEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1290 bp, Artificial Sequence
  Description of Artificial Sequence Synthetic polynucleotide
atgggctggcgagctgctggtgcacttctgctggctctgctgcttcatggcagactgcttgctatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 21 - protein, 21 aa, Artificial Sequence
  Description of Artificial Sequence Synthetic peptide
MGWRAAGALLLALLLHGRLLA

SEQ ID NO: 22 - protein, 18 aa, Artificial Sequence
  Description of Artificial Sequence Synthetic peptide
MKWVTFISLLFLFSSAYS

SEQ ID NO: 23 - protein, 20 aa, Artificial Sequence
  Description of Artificial Sequence Synthetic peptide
MVLQTQVFISLLLWISGAYG

SEQ ID NO: 24 - protein, 23 aa, Artificial Sequence
  Description of Artificial Sequence Synthetic peptide
MQMSPALTCLVLGLALVFGEGSA

SEQ ID NO: 25 - protein, 18 aa, Artificial Sequence
  Description of Artificial Sequence Synthetic peptide
MGLAWGLGVLFLMHVCGT

SEQ ID NO: 26 - protein, 31 aa, Artificial Sequence
  Description of Artificial Sequence Synthetic polypeptide
MLRGPGPGLLLLAVQCLGTAVPSTGASKSKR

SEQ ID NO: 27 - protein, 21 aa, Artificial Sequence
  Description of Artificial Sequence Synthetic peptide
MGWRAAGALLLALLLHGRLLA

SEQ ID NO: 28 - protein, 29 aa, Artificial Sequence
  Description of Artificial Sequence Synthetic peptide
MAGRSHPGPLRPLLPLLVVAACVLPGAGG

SEQ ID NO: 29 - protein, 19 aa, Artificial Sequence
  Description of Artificial Sequence Synthetic peptide
MEFGLSWVFLVALFRGVQC

SEQ ID NO: 30 - protein, 22 aa, Artificial Sequence
  Description of Artificial Sequence Synthetic peptide
MKYLLPTAAAGLLLLAAQPAMA

SEQ ID NO: 31 - protein, 23 aa, Artificial Sequence
  Description of Artificial Sequence Synthetic peptide
MRPTWAWWLFLVLLLALWAPARG

SEQ ID NO: 32 - protein, 21 aa, Artificial Sequence
  Description of Artificial Sequence Synthetic peptide
MWWRLWWLLLLLLLLWPMVWA

SEQ ID NO: 33 - protein, 17 aa, Artificial Sequence
  Description of Artificial Sequence Synthetic peptide
MGVKVLFALICIAVAEA

SEQ ID NO: 34 - protein, 24 aa, Artificial Sequence
  Description of Artificial Sequence Synthetic peptide
MPSSVSWGILLLAGLCCLVPVSLA

SEQ ID NO: 35 - protein, 18 aa, Artificial Sequence
  Description of Artificial Sequence Synthetic peptide
MHSSALLCCLVLLTGVRA

SEQ ID NO: 36 - protein, 6 aa, Artificial Sequence
  Description of Artificial Sequence Synthetic peptide
GRGDSP

(19) United States
(12) Patent Application Publication
(10) Pub. No.: US 2024/0307455 A1
(43) Pub. Date: Sep. 19, 2024

(54) COMPOSITIONS, DEVICES AND METHODS FOR TREATING IMMUNE-MEDIATED INFLAMMATORY DISEASES

(71) Applicant: SIGILON THERAPEUTICS, INC., Cambridge, MA (US)

(72) Inventors: Sofia Brites Boss, Boston, MA (US); Christopher P. Hencken, Boston, MA (US); Hozefa Bandukwala, Southborough, MA (US)

(21) Appl. No.: 18/274,123

(22) PCT Filed: Jan. 26, 2022
(86) PCT No.: PCT/US2022/013940

§ 371 Date: Jul. 25, 2023

Related U.S. Application Data
(60) Provisional application No. 63/141,902, filed on Jan. 26, 2021.

(51) Int. Cl. A61K 35/36; A61K 9/48; A61K 31/436; A61K 31/573; A61K 45/06; A61P 37/06; C12N 5/00

(52) U.S. Cl. CPC A61K 35/36; A61K 9/4808; A61K 31/436; A61K 31/573; A61K 45/06; A61P 37/06; C12N 5/0012; C12N 2510/00

(57) ABSTRACT

Described herein are implantable devices comprising cells genetically modified to express and secrete one or more immunomodulatory proteins. The devices and compositions thereof may be useful for treating immune-mediated inflammatory diseases.

### BACKGROUND

Immune-mediated inflammatory diseases (IMIDs) are a group of seemingly unrelated diseases that share common inflammatory pathways and are triggered by, or result in, the dysregulation of innate and adaptive immune system functions. This dysregulation can result in chronic inflammation and autoimmunity. The development and approval of anti-inflammatory biologic products targeting TNF-alpha and various interleukins has provided transformational benefit to patients living with IMIDs such as rheumatoid arthritis and psoriasis. However, these biologics typically require multiple injections over months or years and have not been shown to be particularly effective in several IMIDs such as autoimmune hepatitis (AIH), graft vs host disease (GvHD), inflammatory bowel disease (IBD), hidradenitis suppurativa (HS), systemic lupus erythematosus (SLE), and TNF-refractory arthritis. Thus, additional therapies for treating IMIDs are desirable.

### SUMMARY

Described herein is an implantable device that contains cells genetically modified to express and secrete at least one immunomodulatory protein when the device is implanted into a recipient. The device is configured to shield the cells from the recipient's immune system and mitigate the foreign body response (FBR) (as defined herein) to the implanted device. In an embodiment, the device is capable of delivering the immunomodulatory protein for a sustained time period (e.g., one to several months up to one to several years) after implant into a subject. In an embodiment, the device also contains an extended release formulation of an immunosuppressant (e.g., a small molecule compound, a peptide). The device and extended release formulation are configured to provide continuous release of the immunosuppressant from the device during a release period (e.g., thirty days, sixty days, ninety days) after the device is implanted into a subject. The immunomodulatory protein and any immunosuppressant delivered by the device may be selected to modulate (e.g., inhibit or induce) one or more aspects of the dysregulated immune pathway in an IMID of interest.

In some embodiments, the immunomodulatory protein secreted by the cells is an anti-inflammatory cytokine (e.g., as defined herein), e.g., an interleukin-10 (IL-10) protein, an interleukin-22 (IL-22) protein, an IL-2 mutein protein, or an IL-1Ra protein, each as defined herein.

In other embodiments, the immunomodulatory protein secreted by the cells is an agonist of an immune checkpoint receptor or ligand, e.g., cytotoxic T lymphocyte associated protein 4 (CTLA-4), programmed cell death protein 1 (PD-1), programmed death-ligand 1 (PD-L1), T cell immunoreceptor with Ig and ITIM domains (TIGIT), CD200 Receptor (CD200R1) T cell immunoglobulin and mucin domain 3 (TIM-3), lymphocyte-activation gene 3 (LAG-3), V-domain Ig suppressor of T cell activation (VISTA). In an embodiment, the agonist is an agonist antibody against the checkpoint receptor, or a fusion protein of the extracellular domain (ECD) of the checkpoint molecule and an immunoglobulin molecule. In an embodiment, the agonist is sCTLA-4-Ig, which consists essentially of the extracellular binding domain of CTLA-4 linked to the Fc domain of a human IgG molecule.

In one aspect, a device described herein continuously releases one or more immunomodulatory non-protein compounds. In an embodiment, the compound is an immunosuppressant, e.g., cyclosporine, rapamycin, or triamcinolone hexacetonide (TAH).

In an embodiment, the surface of the device comprises a compound or polymer that mitigates the FBR (as defined herein) to the device (e.g., an afibrotic compound or afibrotic polymer). In an embodiment, an afibrotic polymer comprises a biocompatible, zwitterionic polymer, e.g., as described in WO 2017/218507, WO 2018/140834, or Liu et al., Zwitterionically modified alginates mitigate cellular overgrowth for cell encapsulation, Nature Communications (2019)10:5262. In an embodiment, the compound is a compound of Formula (I):

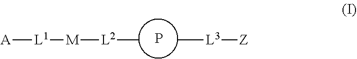

or a pharmaceutically acceptable salt thereof, wherein the variables A, L¹, M, L², P, L³, and Z, as well as related subvariables, are defined herein.

In an embodiment, the device is configured as a two-compartment hydrogel capsule in which an inner compartment comprising the cells expressing the immunomodulatory protein(s), and optionally an immunomodulatory small-molecule compound, is completely surrounded by a barrier compartment. In some embodiments, the barrier compartment comprises a polymer covalently modified with a compound that mitigates the foreign body response (FBR) to the device.

In an embodiment, a device described herein, or a plurality of the device, is combined with a pharmaceutically acceptable excipient to prepare a device preparation or a composition which may be administered to a subject (e.g., into the intraperitoneal cavity) in need of treatment with the immunomodulatory protein(s) produced by the device. In an embodiment, the subject has an IMID. In an embodiment, the subject is a human with AIH or IBD, genetically modified cells are derived from a human cell and the device preparation or composition is capable of continuously delivering a low-dose (as defined herein) of an IL-10 protein or an IL-22 protein for a sustained time period, e.g., at least any of 1 month, 2 months, 4 months or 8 months. In an embodiment, the subject has GvHD and the device preparation or composition is capable of continuously delivering an sCTLA-4-Ig protein.

Also provided herein is a genetically modified mammalian (e.g., human) cell that expresses and secretes one of more of the immunomodulatory proteins described herein. In an embodiment, the immunomodulatory protein is encoded by an exogenous coding sequence inserted into the cell genome at one or more locations. In an embodiment, the exogenous coding sequence is a codon-optimized to achieve higher expression by the cell. In an embodiment, the cell is derived from a human RPE cell, e.g., an ARPE-19 cell.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows the amino acid sequence (SEQ ID NO:1) of an exemplary precursor IL-10 monomer that may be expressed by genetically modified cells described herein, with the signal sequence indicated by underlining.

FIG. 1B shows an exemplary codon-optimized coding sequence (SEQ ID NO:2) for the amino acid sequence in FIG. 1A, with the coding sequence for the signal sequence indicated by shading.

FIG. 1C shows the amino acid sequence (SEQ ID NO:3) of another exemplary precursor IL-10 monomer that may be expressed by genetically modified cells described herein, with the heterologous (HSPG2) signal sequence indicated by underlining.

FIG. 1D and FIG. 1E show exemplary codon-optimized coding sequences (SEQ ID NO:4 and SEQ ID NO:5) for the amino acid sequence in FIG. 1C, with the coding sequence for the signal sequence indicated by shading.

FIG. 1F shows the amino acid sequence (SEQ ID NO:6) of an exemplary variant of precursor IL-10 monomer that may be expressed by genetically modified cells described herein, with the signal sequence indicated by underlining.

FIG. 1G shows an exemplary codon-optimized coding sequence for the amino acid sequence in FIG. 1F (SEQ ID NO:7), with the coding sequence indicated by shading.

FIG. 2A shows the amino acid sequence (SEQ ID NO:8) of an exemplary precursor IL-22 protein that may be expressed by genetically modified cells described herein, with the signal sequence indicated by underlining.

FIG. 2B shows an exemplary coding sequence (SEQ ID NO:9) for the amino acid sequence in FIG. 2A.

FIG. 2C show an exemplary codon-optimized coding sequence (SEQ ID NO:10) for the amino acid sequence in FIG. 2A.

FIG. 2D shows the amino acid sequence (SEQ ID NO:11) of an exemplary precursor IL-22 protein that may expressed by genetically modified cells described herein, with the heterologous (HSPG2) signal sequence indicated by underlining and the extracellular domain indicated with double underlining.

FIG. 2E shows an exemplary codon-optimized coding sequence (SEQ ID NO: 12) for the amino acid sequence in FIG. 2D.

FIG. 3A shows the amino acid sequence (SEQ ID NO: 13) of the membrane-bound isoform of human precursor CTLA-4 protein, with the signal sequence indicated by underlining and the extracellular domain indicated with shading.

FIG. 3B shows the amino acid sequence (SEQ ID NO: 14) for an exemplary precursor sCTLA-4-Ig fusion protein that may be expressed by genetically modified cells described herein.

FIG. 3C shows an exemplary coding sequence (SEQ ID NO:15) for the amino acid sequence in FIG. 3B.

FIG. 3D shows the amino acid sequence (SEQ ID NO: 16) of another exemplary precursor sCTLA-4-Ig fusion protein that may be expressed by genetically modified cells described herein, with the signal sequence indicated by underlining and the extracellular domain indicated with shading.

FIG. 3E shows an exemplary coding sequence (SEQ ID NO:17) for the amino acid sequence in FIG. 3D.

FIG. 3F shows the amino acid sequence (SEQ ID NO:18) of an exemplary precursor soluble CTLA-4 protein that may be expressed by genetically modified cells described herein.

FIG. 4A illustrates plasma levels of IL-10 in mice implanted intraperitoneally with hydrogel capsules encapsulating cells genetically modified to express and secrete human IL-10 (labeled IL-10 Spheres—line above baseline) or capsules without cells (labeled Control Spheres—line at baseline).

FIG. 4B illustrates plasma levels of human IL-10 in mice implanted intraperitoneally with empty capsules (Control), with hydrogel capsules encapsulating cells genetically modified to express and secrete human IL-10 (middle bar), or with the IL-10 producing capsules and treated with an IL-10 receptor blocking antibody (right bar).

FIG. 4C, FIGS. 4D, 4E and 4F illustrate various therapeutic effects of exemplary IL-10 producing devices of the disclosure in a mouse model of AIH.

FIGS. 5A-5C illustrate the biological activity of an exemplary human IL-22 protein secreted by genetically modified ARPE-19 cells. FIG. 5A shows recombinant IL-22 induced IL-10 production from Colo25 cells. FIG. 5B shows that IL-22 secreted from genetically modified ARPE-19 cells induces IL-10 production from Colo25 cells. FIG. 5C shows that IL-10 induced by IL-22 secreted from genetically modified ARPE-19 cells is inhibited by addition of IL-22 BP. In FIG. 5C, the ARPE WT supernatant (control) samples are shown on the left and the ARPE IL-22 supernatant samples are shown on the right.

FIGS. 6A, 6B and 6C illustrate continuous delivery of a soluble CTLA-4 protein and an immunosuppressant by exemplary hydrogel capsules described herein to prevent pathogenic graft versus host responses in a xenogeneic model of GvHD.

FIG. 7A shows the amino acid sequence (SEQ ID NO:19) of an exemplary precursor PD-L1-Fc protein that may expressed by genetically modified cells described herein, with the signal sequence indicated by underlining and the Fc portion indicated by shading.

FIG. 7B shows an exemplary codon-optimized coding sequence (SEQ ID NO:20) for the amino acid sequence in FIG. 7A.

### DETAILED DESCRIPTION

The present disclosure features an implantable device capable of continuous delivery of at least one immunomodulatory protein to a subject. The immunomodulatory protein is expressed and secreted by living cells contained in the device. A variety of device configurations and their use for treating IMIDs are contemplated by the present disclosure. Various embodiments will be described below.

### Abbreviations and Definitions

Throughout the detailed description and examples of the disclosure the following abbreviations will be used.
CM-Alg chemically modified alginate
CM-LMW-Alg chemically modified, low molecular weight alginate
CM-LMW-Alg-101 low molecular weight alginate, chemically modified with Compound 101 shown in Table 4
CM-HMW-Alg chemically modified, high molecular weight alginate
CM-HMW-Alg-101 high molecular weight alginate, chemically modified with Compound 101 shown in Table 4
CM-MMW-Alg chemically modified, medium molecular weight alginate
CM-MMW-Alg-101 medium molecular weight alginate, chemically modified with Compound 101 shown in Table 4
HMW-Alg high molecular weight alginate
MMW-Alg medium molecular weight alginate
U-Alg unmodified alginate
U-HMW-Alg unmodified high molecular weight alginate
U-LMW-Alg unmodified low molecular weight alginate
U-MMW-Alg unmodified medium molecular weight alginate
70:30 CM-Alg:U-Alg 70:30 mixture (V:V) of a chemically modified alginate and an unmodified alginate, e.g., as described in WO2020069429.

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” means when used herein to modify a numerically defined parameter (e.g., a physical description of a hydrogel capsule such as diameter, sphericity, number of cells encapsulated therein, the number of capsules in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer”, “administering”, or “administration”, as used herein, refer to implanting, absorbing, ingesting, injecting or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Table 4) is lower than the FBR induced by implantation of an afibrotic-null reference device, i.e., a device that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Anti-inflammatory cytokine”, as used herein, refers to a naturally-occurring cytokine, as well that exhibits one or more anti-inflammatory and/or immunosuppressive activities in the regulation of the immune system, as well as variants thereof (e.g., modified amino acid sequence, fusion proteins) that have of the naturally-occurring cytokine. An anti-inflammatory cytokine may either inhibit pro-inflammatory cytokine synthesis or control pro-inflammatory cytokine-mediated cellular activities. Anti-inflammatory cytokines that may be produced by, or induced by, devices and compositions described herein include, but are not limited to, interleukin-4 (IL-4), interleukin-5 (IL-5), interleukin-6 (IL-6), interleukin-10 (IL-10) (and its family members, including IL-19, IL-22, IL-24, and IL-26), interleukin-11 (IL-11), interleukin-12 (IL-12), interleukin-13 (IL-13), interleukin-27 (IL-27), interleukin-35 (IL-35), interleukin-33 (IL-33), interleukin-37 (IL-37), interferon beta and transforming growth factor-beta (TGF-ß).

“Autoimmune hepatitis” and “AIH”, as used herein, refer to a non-contagious, chronic, inflammatory autoimmune disease in which a subject's immune system attacks liver cells. The pathology of AIH results from a breakdown in immune tolerance leading to production of pro-inflammatory cytokines by autoreactive T-cells. This process leads to persistent inflammation in the liver that can result in scarring, cirrhosis, liver failure requiring a liver transplant, and death. There are two clinically relevant types of AIH: type 1, typically diagnosed in adulthood, and type 2, diagnosed during childhood. AIH occurs more frequently in females than males and is commonly associated with other autoimmune conditions including type 1 diabetes, Hashimoto's thyroiditis, and celiac disease.

“Cell,” as used herein, refers to a genetically modified cell or a cell that is not genetically modified. In an embodiment, a cell is an immortalized cell or a genetically modified cell derived from an immortalized cell. In an embodiment, the cell is a live cell, e.g., is viable as measured by any technique described herein or known in the art.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface | Asp, Glu, Asn, Aln, His, Arg, Lys |
| Exposure | Cys, Ser, Tyr, Pro, Ala, Gly, Trp, Tyr |
| | Met, Ile, Leu, Val, Phe |
| Secondary Structure | Ala, Glu, Aln, His, Lys, Met, Leu, Arg |
| Propensity | Cys, Thr, Ile, Val, Phe, Tyr, Trp |
| | Ser, Gly, Pro, Asp, Asn |
| Evolutionary Conservation | Asp, Glu |
| | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, an immunomodulatory protein that consists essentially of a recited amino acid sequence may also include one or more amino acids, including substitutions in the recited amino acid sequence, of one or more amino acid residues, which do not materially affect the relevant biological activity of the immunomodulatory protein.

“CTLA-4” and “CTLA4” refers to Cytotoxic T-Lymphocyte Antigen 4, also known as CD 152 (Cluster of differentiation 152), a protein that binds via its extracellular domain to costimulatory ligands B7-1 (CD80) and B7-2 (CD86) on the surface of antigen presenting cells (APCs). CLTA-4 inhibits immune response in two principal ways: it competes with CD28 for binding to B7-1 and B7-2 and thereby blocks co-stimulation, and it negatively signals to inhibit T cell activation.

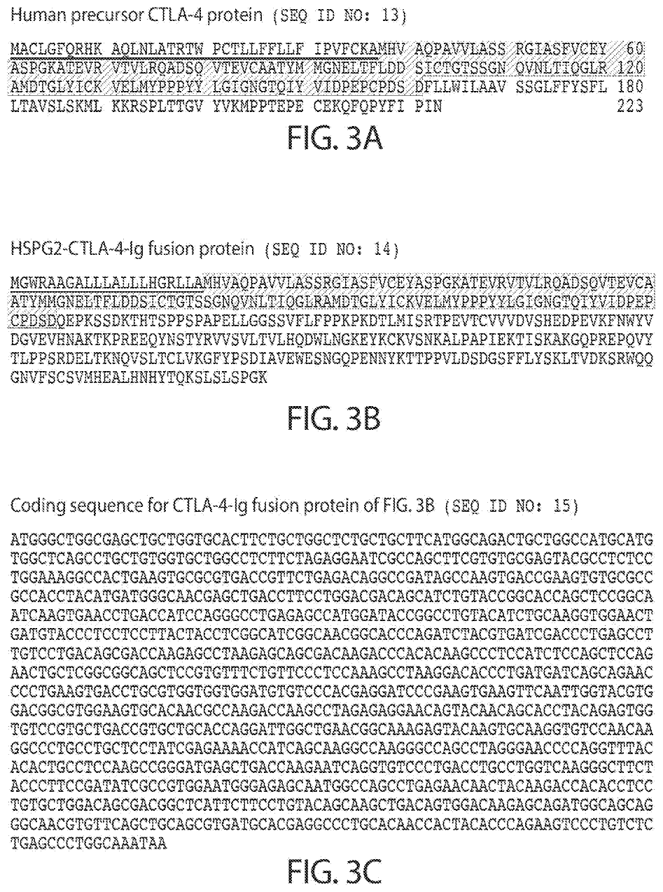

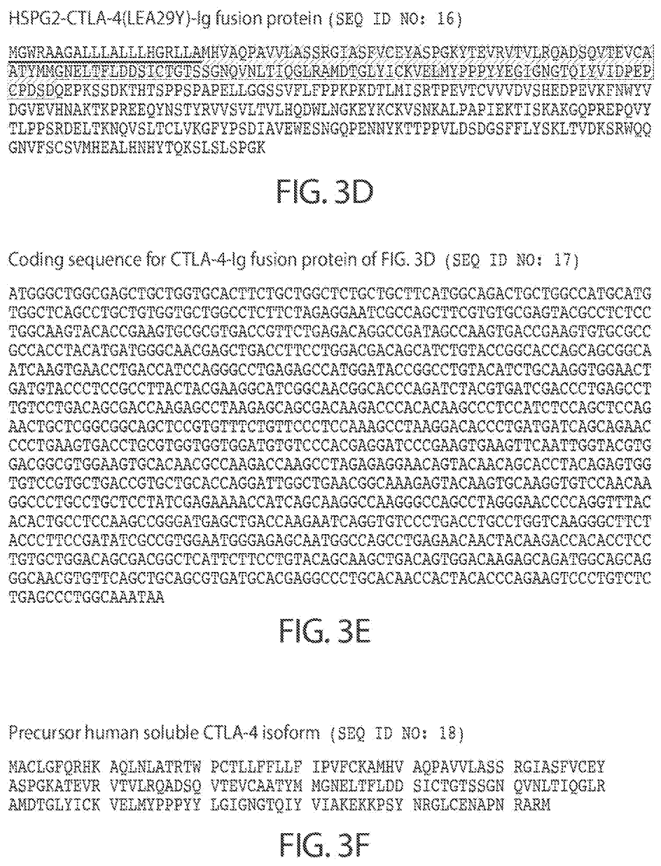

“Soluble CTLA-4” and “sCTLA-4”, as used herein, refers to a secreted protein (e.g., lacks an operable transmembrane domain), which comprises the CTLA-4 extracellular domain (ECD) or portions thereof that bind B7-1 and/or B7-2. In an embodiment, the CTLA-4 ECD is from a mammalian CTLA-4 protein (e.g., human CTLA-4) or a variant thereof. In an embodiment, a soluble CTLA-4 protein comprises the ECD of human CTLA-4 (e.g., amino acids 38-161 of FIG. 3A) or a variant thereof (e.g., comprising one or more amino acid substitutions). A sCTLA-4 protein comprising a variant of a mammalian CTLA-4 ECD amino acid sequence retains the ability to bind to one or more of B7-1 and/or B7-2 at substantially the same or greater avidity as a protein comprising the wild-type mammalian CTLA-4 ECD sequence. In an embodiment, an sCTLA-4 protein comprises any of the CTLA-4 variants disclosed in EP3029062A1 or WO201103584A2. In an embodiment, an sCTLA-4 protein comprises the amino acid sequence shown in FIG. 3F or a variant thereof.

“sCTLA-4 fusion protein”, as used herein, refers to an sCTLA-4 protein operably linked to all or a portion of a heterologous protein that confers a beneficial property, e.g., higher expression, better stability, longer half-life in vivo. Examples of sCTLA-4 fusion proteins are described in United States patent application publication US 2014/0147418 A1.

“CTLA-4-Ig fusion protein”, as used herein, is a sCTLA-4 fusion protein in which the heterologous component of the fusion comprises a mammalian immunoglobulin protein (IgG) or a portion thereof (e.g., Fc region). In an embodiment, the CTLA-4 amino acid sequence is linked to the amino acid sequence of the IgG Fc region via a linker, e.g., a single glutamine residue (as described in EP3029062A1) or any of the linkers described in WO201103584.

In some embodiments, the cells in a device described herein are genetically modified to express a precursor CTLA-4-Ig protein that comprises a signal sequence operably linked to the N-terminus of the mature CTLA-4 component. The signal sequence may be any sequence that will permit secretion of the precursor fusion protein. In an embodiment, the signal sequence is from a mammalian CTLA-4 (e.g., a human CTLA-4, e.g., amino acids 1-37 of FIG. 3A) In another embodiment, the signal sequence is from a secreted protein, e.g., the signal sequence from human HSPG2 (e.g., amino acids 1-21 of FIG. 3B).

“Derived from”, as used herein with respect to cells, refers to cells obtained from tissue, cell lines, or cells, which optionally are then cultured, passaged, differentiated, induced, etc. to produce the derived cells. For example, mesenchymal stem cells can be derived from mesenchymal tissue and then differentiated into a variety of cell types.

“Device”, as used herein, refers to any implantable object (e.g., a particle, a hydrogel capsule, an implant, a medical device), which contains a cell or cells (e.g., live cells) capable of expressing and secreting an immunomodulatory protein or immunomodulatory proteins following implant of the device, and has a configuration that supports the viability of the cells by allowing cell nutrients to enter the device.

“Differential volume,” as used herein, refers to a volume of one compartment within a device described herein that excludes the space occupied by another compartment(s). For example, the differential volume of the second (e.g., outer) compartment in a 2-compartment device with inner and outer compartments, refers to a volume within the second compartment that excludes space occupied by the first (inner) compartment.

“Endogenous nucleic acid” as used herein, is a nucleic acid that occurs naturally in a subject cell.

“Endogenous polypeptide,” as used herein, is a polypeptide that occurs naturally in a subject cell.

“Exogenous nucleic acid,” as used herein, is a nucleic acid that does not occur naturally in a subject cell.

“Exogenous polypeptide,” as used herein, is a polypeptide that does not occur naturally in a subject cell, e.g., genetically modified cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Extended-release formulation”, as used herein, means a formulation that releases an immunosuppressant (as defined herein) from a device in a sustained-release (SR) or controlled-release (CR) profile. SR maintains release of the immunosuppressant over a sustained period but not at a constant rate. CR maintains release of the immunosuppressant over a sustained period at a nearly constant rate. In some embodiments, extended release refers to SR over a period of at least 30 days, at least 45 days, at least any of 50, 60, 70, 80, 90, 100, 110, 120 days or more.

“Genetically-modified cell,” as used herein, is a cell (e.g., an RPE cell) having a non-naturally occurring alteration, and typically comprises a nucleic acid sequence (e.g., an exogenous DNA or RNA) or a polypeptide not present (or present at a different level than) in an otherwise similar cell under similar conditions that is not genetically modified (e.g., lacks the exogenous nucleic acid sequence). In an embodiment, a genetically modified cell comprises an exogenous nucleic acid (e.g., a vector or an altered chromosomal sequence). In an embodiment, a genetically modified cell comprises an exogenous polypeptide. In an embodiment, a genetically modified cell comprises an exogenous nucleic acid sequence, e.g., a sequence, e.g., DNA or RNA, not present in a similar cell that is not genetically modified. In an embodiment, the exogenous nucleic acid sequence is chromosomal, e.g., the exogenous nucleic acid sequence is an exogenous sequence disposed in endogenous chromosomal sequence. In an embodiment, the exogenous nucleic acid sequence is chromosomal or extra chromosomal, e.g., a non-integrated vector. In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a chromosomal or extra-chromosomal nucleic acid sequence, which comprises a sequence that encodes a polypeptide, or which is expressed as a polypeptide. In an embodiment, the exogenous nucleic acid sequence comprises a first chromosomal or extra-chromosomal exogenous nucleic acid sequence that modulates the conformation or expression of a second nucleic acid sequence, wherein the second amino acid sequence can be exogenous or endogenous. For example, a genetically modified cell can comprise an exogenous nucleic acid that controls the expression of an endogenous sequence. In an embodiment, a genetically modified cell comprises a polypeptide present at a level or distribution which differs from the level found in a similar cell that has not been genetically modified. In an embodiment, a genetically modified cell comprises an RPE genetically modified to produce an RNA or a polypeptide. For example, a genetically modified cell may comprise an exogenous nucleic acid sequence comprising a chromosomal or extra-chromosomal exogenous nucleic acid sequence that comprises a sequence which is expressed as RNA, e.g., mRNA or a regulatory RNA. In an embodiment, a genetically modified cell (e.g., an RPE cell) comprises an exogenous nucleic acid sequence that comprises a chromosomal or extra-chromosomal nucleic acid sequence comprising a sequence that encodes a polypeptide, or which is expressed as a polypeptide. In an embodiment, the polypeptide is encoded by a codon optimized sequence to achieve higher expression of the polypeptide than a naturally-occurring coding sequence. The codon optimized sequence may be generated using a commercially available algorithm, e.g., GeneOptimizer (ThermoFisher Scientific), OptimumGene™ (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl_2, pp. W526-W531 (2005)). In an embodiment, a genetically modified cell (e.g., an RPE cell) comprises an exogenous nucleic acid sequence that modulates the conformation or expression of an endogenous sequence. In an embodiment, a genetically modified cell (e.g., RPE cell) is cultured from a population of stably-transfected cells, or from a monoclonal cell line.

“Glucocorticoid”, as used herein, means a naturally occurring or synthetic compound (e.g., hormone, small molecule) which binds to the glucocorticoid receptor expressed by mammalian cells (e.g., human cells), which binding results in up-regulation of the expression of anti-inflammatory proteins (e.g. TGF-beta, interleukin (IL)-1 receptor antagonist, IL-4, IL-10, IL-11, IL-13) and down-regulation of the expression of pro-inflammatory proteins (e.g., interferon gamma, granulocyte-macrophage stimulating factor (GM-CSF), IL-1, IL-12, tumor necrosis factor alpha (TNF-, MCP-1). Non-limiting examples of glucocorticoids include triamcinolone and triamcinolone derivatives (e.g., triamcinolone hexacetonide (TAH), triamcinolone acetonide, triamcinolone benetonide, triamcinolone diacetate), fluticasone and fluticasone derivatives (e.g., fluticasone furoate, fluticasone propionate), mometasone and mometasone derivatives (e.g., mometasone furoate), clobetasol and clobetasol derivatives (e.g., clobetasol propionate), beclomethasone and beclomethasone derivatives (e.g., beclomethasone dipropionate), prednisone, prednisolone, methylprednisolone, hydrocortisone, betamethasone, and dexamethasone. In an embodiment, the glucocorticoid is a compound of Formula (IV), as defined herein. In some embodiments, the glucocorticoid is not dexamethasone, prednisone, methylprednisolone, prednisolone, hydrocortisone, or fludrocortisone. In some embodiments, the glucocorticoid is not triamcinolone, triamcinolone acetonide, or clobetasol propionate.

“Graft vs Host Disease” and “GvHD”, as used herein, refer to an acute or chronic condition characterized by inflammation in different organs, resulting from donor immune cells in a transplant (graft) (or immune cells derived from such donor immune cells) recognizing the recipient (host) cells and tissues as foreign and mounting a pathogenic inflammatory response. GvHD is most commonly associated with non-autologous (allogeneic) bone marrow and stem cell transplants (e.g., hematopoietic stem cell transplant (HSCT)), but may also result from other types of transplanted tissues, including solid organ transplants.

“Immunosuppressant compound” or “immunosuppressant” as used herein, refers to a compound other than a protein that inhibits or prevents an activity of the immune system. Non-limiting examples of immunosuppressants that may be released by devices and compositions described herein include: (i) compounds that act on immunophilins (e.g., cyclosporine, everolimus, rapamycin, tacrolimus, zotarolimus); (ii) corticosteroids, including synthetic glucocorticoids (e.g., TAH, fluticasone furoate, fluticasone propionate, mometasone furioate); and (iii) cytostatics (e.g., azathioprine, mercaptopurine, cyclophosphamide, methotrexate).

“Inflammatory bowel disease” and “IBD”, as used herein, refer to a disorder or condition characterized by chronic inflammation in part or all of the gastrointestinal (GI) tract. While the exact cause of IBD is unknown, it is a result of dysregulation in the immune system and includes both autoimmune and immune-mediated phenomena. The two most common IBDs are Crohn's disease (CD), which can affect any part of the GI tract (e.g., mouth to anus, but most commonly the small intestine), and ulcerative colitis (UC), which affects the large intestine (colon) and the rectum.

“Interleukin-1 receptor antagonist protein” and “IL-1Ra protein”, as used herein, refer to a protein that specifically inhibits IL-1 mediated inflammation and comprises the amino acid sequence of a mammalian (e.g., human) precursor or mature IL-1Ra protein or a variant thereof. IL-1Ra is structurally related to IL-1 and is capable of binding to IL-1RI but fails to interact with IL-1RAcP. Thus, IL-1Ra inhibits the pro-inflammatory effects of IL-1 by functioning as a competitive inhibitor in receptor binding. In some embodiments, an IL-1Ra protein produced by a device described herein comprises an amino acid sequence that is a variant of a mammalian IL-1Ra amino acid sequence, e.g., a human precursor or mature IL-1Ra sequence, provided that the resulting variant IL-1Ra protein exhibits an activity that is comparable to or greater than the corresponding activity exhibited by the wild-type mammalian IL-1Ra protein. The wild-type human IL-1Ra is expressed as a 177 amino acid precursor polypeptide with a 25 amino acid signal sequence (UniProtKB/Swiss-Prot: P18510.1) Exemplary variants of human ILIRa amino acid sequence are described in U.S. Pat. No. 7,619,066 and in WO 2008/132485.

“Interleukin-2 mutein protein” and “IL-2 mutein protein”, as used herein, refer to a protein that preferentially stimulates Treg cells to suppress autoimmune inflammation and comprises a mutant of the amino acid sequence of a mammalian (e.g., human) precursor or mature IL-2 protein. The wild-type human IL-2 is expressed as a 153 amino acid precursor polypeptide with a 20 amino acid signal sequence (NCBI Reference Sequence: NP_000577.2). An IL-2 mutein useful as an immunomodulatory protein promotes the proliferation, survival, activation and/or function of CD3+FoxP3+ T cells over CD3+FoxP3− T cells. Exemplary IL-2 muteins are described in WO 2021/119093, WO 2020/30602, WO 2019/112852, WO 2019/112854, WO 2019/104092, WO 2018/217989, WO 2016/164937, WO 2016/025385, WO 2016/014428, WO2014153111, and WO 2010/085495.

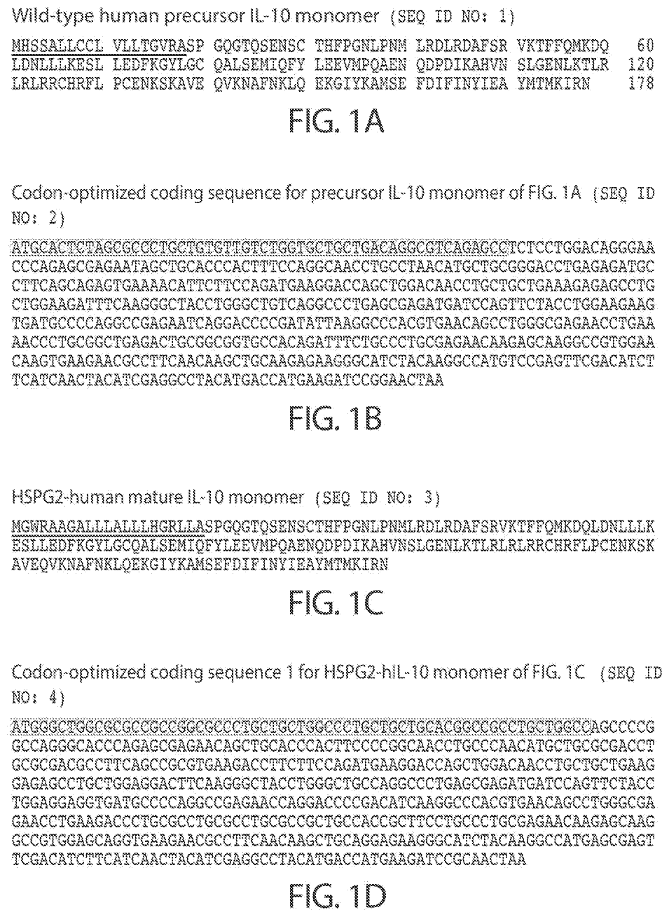

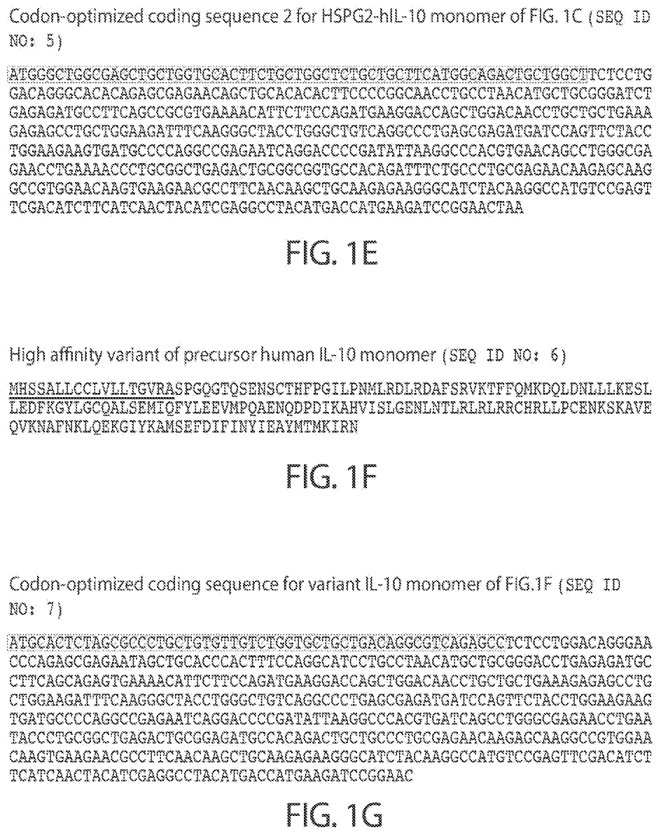

“Interleukin-10 protein” and “IL-10 protein”, as used herein, refer to a dimeric protein comprising two, non-covalently joined monomers, which becomes biologically inactive upon disruption of the non-covalent interactions between the two monomers. In some embodiments, each monomer in an IL-10 protein produced by a device described herein comprises an amino acid sequence that is identical to the amino acid sequence of a mammalian IL-10 monomer, e.g., human IL-10. In an embodiment, the IL-10 amino acid sequence in one or both of the monomers is a variant of a mammalian IL-10 amino acid sequence, provided that the resulting homodimer exhibits an activity that is comparable to or greater than the corresponding activity exhibited by the wild-type mammalian IL-10 protein. In an embodiment, a device described herein produces a high affinity variant of hIL-10, e.g., in which each of the monomers comprises amino acids 19-170 shown in FIG. 1F. IL-10 activity is described in, e.g., U.S. Pat. No. 5,231,012 and in International Patent Publication Nos. WO 97/42324 and WO 2014/023673, which provide in vitro assays suitable for measuring such activity. In particular, IL-10 inhibits the synthesis of at least one cytokine in the group consisting of IFN-γ, lymphotoxin, IL-2, IL-3, and GM-CSF in a population of T helper cells induced to synthesize one or more of these cytokines by exposure to antigen and antigen presenting cells (APCs). In an embodiment, one or both of the monomers in the IL-10 protein comprises an amino acid sequence from a different protein (e.g., an IgG Fc region, albumin) operably linked to the N-terminus or C-terminus of the IL-10 amino acid sequence.

In some embodiments, the cells in a device described herein are genetically modified to encode a precursor IL-10 monomer, which comprises a signal sequence operably linked to the N-terminus of a mammalian mature IL-10 amino acid sequence of 160 amino acids (or a variant thereof) which includes two pairs of cysteine residues that form two intramolecular disulfide bonds. The wild-type human IL-10 (hIL-10) monomer comprises the mature amino acid sequence set forth in FIG. 1A. The signal sequence may be any sequence that will permit secretion of the precursor protein. In an embodiment, the monomer polypeptide comprises the signal sequence from a mammalian IL-10 monomer, e.g., amino acids 1-18 of FIG. 1A. In another embodiment, the signal sequence is from a heterologous secreted protein, e.g., the signal sequence from human HSPG2 (e.g., amino acids 1-21 of FIG. 1C).

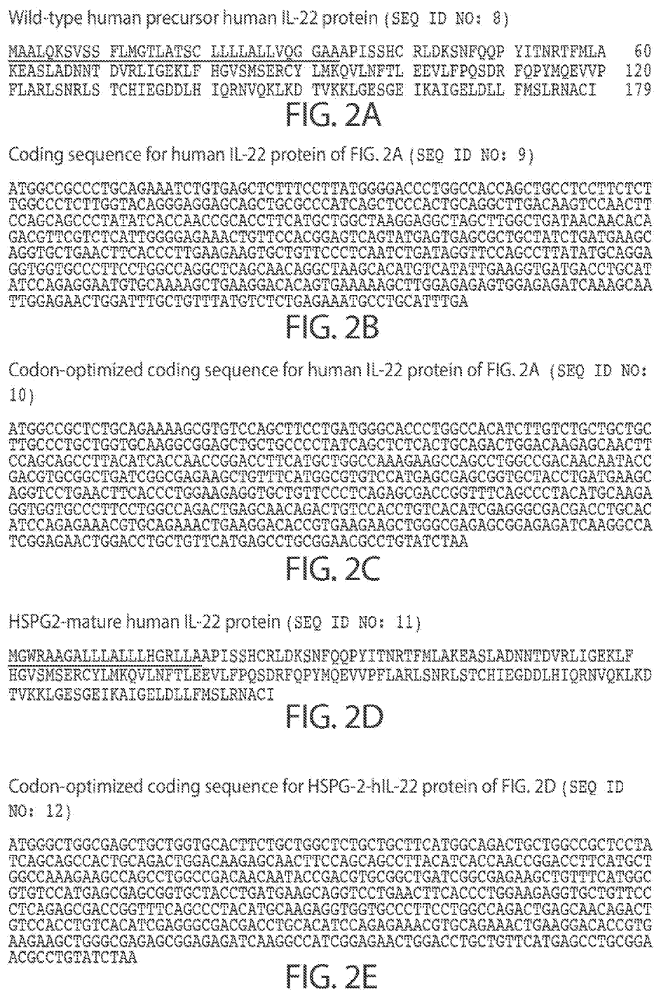

“Interleukin-22 protein” or “IL-22 protein”, as used herein, refers to a protein comprising the amino acid sequence of a mammalian precursor or mature IL-22 or a variant thereof. The active, secreted form of wild-type human IL-22 is a 146 amino acid monomer protein which signals through a heterodimeric receptor comprised of IL-10R2 subunit and IL-22R1 subunit. The wild-type precursor human IL-22 has the 179 amino acid sequence shown in FIG. 2A. In some embodiments, an IL-22 protein produced by a device described herein comprises an amino acid sequence that is a variant of a mammalian IL-22 amino acid sequence, e.g., a human precursor or mature IL-22 sequence, provided that the resulting variant IL-22 protein exhibits an activity that is comparable to or greater than the corresponding activity exhibited by the wild-type mammalian IL-22 protein. In an embodiment, the IL-22 protein may comprise two monomer subunits, with each subunit comprising the amino acid sequence of a mammalian IL-22 (e.g., hIL-22) or variant thereof. Exemplary IL-22 dimers are described in US published patent applications 20130171100 and US20160287670. In an embodiment, the IL-22 protein comprises an amino acid sequence from a different protein (e.g., an IgG Fc region, albumin) operably linked to the N-terminus or C-terminus of the IL-22 amino acid sequence.

In some embodiments, the cells in a device described herein are genetically modified to express a precursor IL-22 protein that comprises a signal sequence operably linked to the N-terminus of a mammalian mature IL-22 amino acid sequence (e.g., amino acids 34-179 of FIG. 2A). In an embodiment, the monomer polypeptide comprises the signal sequence from a mammalian IL-22, e.g., amino acids 1-33 of FIG. 2A. In another embodiment, the signal sequence is from a heterologous secreted protein, e.g., the signal sequence from human HSPG2 (e.g., amino acids 1-21 of FIG. 2C).

“Programmed death ligand-1 protein” and “PD-L1 protein”, as used herein, refer to a protein that interacts with programmed cell death protein 1 (PD-1) to suppress immune response against autoantigens. Precursor human PD-L1 is 290 amino acids in length with amino acids 1 to 18 constituting the signal sequence, amino acids 19-238 forming the extracellular domain (ECD), and amino acids 239-259 and 260-290 forming the transmembrane and cytoplasmic domains, respectively (UniProtKB/Swiss-Prot: Q9NZQ7.1).

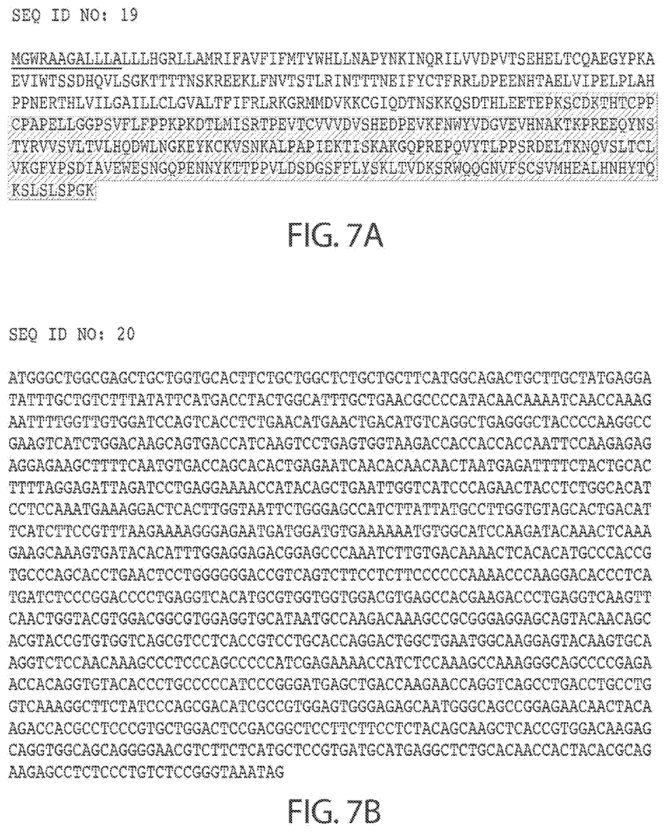

“Soluble PD-L1” and “sPD-L1”, as used herein, refers to a secreted protein (e.g., lacks an operable transmembrane domain), which comprises the amino acid sequence of the extracellular domain (ECD) of programmed death ligand-1 protein. PD-L1 interacts with programmed cell death protein 1 (PD-1) to suppress immune response against autoantigens. Precursor human PD-L1 is 290 amino acids in length with amino acids 1 to 18 constituting the signal sequence, amino acids 19-238 forming the extracellular domain (ECD), and amino acids 239-259 and 260-290 forming the transmembrane and cytoplasmic domains, respectively (UniProtKB/Swiss-Prot: Q9NZQ7.1). In an embodiment, the ECD in a sPD-L1 is from a mammalian PD-L1 protein (e.g., human PD-L1) or a variant thereof. In an embodiment, a soluble PD-L1 protein comprises the ECD of human PD-L1 (e.g., amino acids 19-238 or 19-239 of UniProtKB/Swiss-Prot: Q9NZQ7.1) or a variant thereof (e.g, comprising one or more amino acid substitutions) A sPD-L1 protein comprising a variant of a mammalian PD-L1 ECD amino acid sequence retains the ability to bind to one or more of PD-1 at substantially the same or greater avidity as a protein comprising the wild-type mammalian PD-L1 ECD sequence. In an embodiment, an sPD-L1 protein is secreted as part of a fusion protein that comprises the Fc region of an IgG, e.g., IgG1 In an embodiment, a SPD-L1 protein delivered by a device described herein is a PD-L1-Fc fusion protein that consists essentially of, or consists of, amino acids 22 to 197 of the precursor amino acid sequence shown in FIG. 7A.

“Peptide”, as used herein, is a polypeptide of less than 50 amino acids, typically, less than 25 amino acids.

“Polymer composition”, as used herein, is a composition (e.g., a solution, mixture) comprising one or more polymers. As a class, “polymers' includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, is a polymer comprising amino acid residues linked through peptide bonds and having at least two, and in some embodiments, at least 10, 50, 75, 100, 150 or 200 amino acid residues.

“Prevention,” “prevent,” and “preventing”, as used herein, refer to a treatment that comprises administering or applying a therapy, e.g., administering a composition of devices encapsulating cells (e.g., as described herein), prior to the onset of a disease, disorder, or condition to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

“Protein”, as used herein, comprises one or more polypeptide chains of at least 50 amino acids in length. In an embodiment, a protein has two or more polypeptide chains have identical or non-identical amino acid sequences of at least 50 amino acids in length. In an embodiment, the polypeptide chains in a protein are noncovalently associated or covalently joined, e.g., via disulfide bond(s).

“Proinflammatory response”, as used herein includes increased expression of one or more cytokines that contribute to inflammation in an autoimmune disease or other IMID of interest. In an embodiment, a pro-inflammatory response is an increased expression of one or more pro-inflammatory cytokines selected from the group consisting of IL-1, IL-2, IL-6, IL-17, IL-18, IL-23, IL-27, IL-32, IL-33, interferon (IFN)-γ, type 1 interferon-alpha, tumor necrosis factor (TNF)-α, granulocyte-macrophage colony-stimulating factor (GM-csf).

“RPE cell”, as used herein, refers to a cell having one or more of the following characteristics: a) it comprises a retinal pigment epithelial cell (RPE) (e.g., cultured using the ARPE-19 cell line (ATCC® CRL-2302™)) or a cell derived therefrom, e.g., by stably transfecting cells cultured from the ARPE-19 cell line with an exogenous sequence that encodes an immunomodulatory protein or otherwise engineering such cultured ARPE-19 cells to express an exogenous protein or other exogenous substance, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or aB-crystallin; ii) it does not express one or more of the biomarkers CRALBP, RPE-65, RLBP, BEST1, or aB-crystallin; iii) it is naturally found in the retina and forms a monolayer above the choroidal blood vessels in the Bruch's membrane; iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). In an embodiment, an RPE cell described herein is genetically modified, e.g., to have a new property, e.g., the cell is genetically modified to express and secrete one or more immunomodulatory proteins or an immunomodulatory agent. In other embodiments, an RPE cell is not genetically modified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, means a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a device (e.g., a hydrogel capsule or other particle), means the device has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female), e.g., of any age group, a pediatric subject (e.g., infant, child, adolescent) or adult subject (e.g., young adult, middle-aged adult, or senior adult). In an embodiment, the subject is a non-human animal, for example, a mammal (e.g., a mouse, a dog, a primate (e.g., a cynomolgus monkey or a rhesus monkey)). In an embodiment, the subject is a commercially relevant mammal (e.g., a cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of a disease, disorder, or condition. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, a therapy (e.g., a device composition) may be administered to a susceptible individual prior to the onset of symptoms (e.g., considering a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₄ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents, e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₅ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₅ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to:—CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′- and —R′C(O)₂—.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including 1H, 2H (D or deuterium), and 3H (T or tritium); C may be in any isotopic form, including ¹²C, ¹³C, and ¹⁴C; O may be in any isotopic form, including 160 and 180; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (I) in a prodrug form. Prodrugs are those compounds that readily undergo chemical changes under physiological conditions to provide the compounds useful for preparing devices in the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or surface of an implantable device, e.g., a particle, a hydrogel capsule. The connection represented by “ [symbol] ” may refer to direct attachment to the entity, e.g., a polymer or an implantable element, may refer to linkage to the entity through an attachment group. An “attachment group,” as described herein, refers to a moiety for linkage of a compound of Formula (I) to an entity (e.g., a polymer or an implantable element (e.g., a device) as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S, —S(O)ₓ—, —OS(O)ₓ—, —N(R^{C})S(O)ₓ—, —S(O)\N(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl. In some embodiments, an attachment group is a cross-linker. In some embodiments, the [symbol] attachment group is —C(O)(C₁-C₆¹, and R¹ is as described herein [symbol] . In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Genetically Modified Cells and Immunomodulatory Proteins

Devices of the present disclosure contain (e.g., encapsulate) cells genetically modified to express and secrete at least one immunomodulatory protein that has a biological activity or biological effect useful for treating an IMID of interest. In an embodiment, the immunomodulatory protein to be expressed and secreted is chosen based on an established or probable etiology of one or more symptoms typically exhibited by patients with the IMID to be treated.

Exemplary immunomodulatory proteins that may be secreted by a genetically modified cell described herein include: human IL-10 and fusions thereof (e.g., IL-10-Fc); human IL-22 and fusions thereof (e.g., IL-22-Fc); human IL-1Ra and fusions thereof (e.g., IL-1Ra-Fc); soluble human CTLA4 and fusions thereof (e.g., CTLA4-Ig, CTLA4-Fc); human IL-2 and variants thereof (e.g., contain mutations that decrease affinity for the CD22 receptor and/or mutations that increase affinity for CD25), and fusion proteins comprising IL-2 or an IL-2 variant; soluble forms of human PD-L1 and fusions thereof (e.g., PD-L1-Fc).

The immunomodulatory protein may be expressed as part of a fusion protein, which comprises a first amino acid sequence which forms a therapeutic domain (e.g., has substantially the same activity as the immunomodulatory protein) and a second amino acid sequence with a desired property. In an embodiment, the second amino acid sequence facilitates secretion of the fusion protein by the encapsulated cells, e.g., a signal peptide sequence from a heterologous protein. In an embodiment, the second amino acid sequence forms a tissue-targeting moiety, e.g., binds to a tissue-specific protein of interest, e.g., a blood-brain barrier transporter, a liver targeting moiety, a tumor targeting moiety. In an embodiment, the second amino acid sequence forms a half-life-extending domain, e.g., an immunoglobulin Fc, albumin, a serum albumin binding domain. In an embodiment, the fusion protein comprises amino acid sequences for both a tissue-targeting domain and a half-life extending domain in addition to the amino acid sequence for the therapeutic domain.

The genetically modified cell(s) may be derived from a variety of different cell types (e.g., human cells), including adipose cells, epidermal cells, epithelial cells, endothelial cells, fibroblast cells, islet cells, mesenchymal stem cells, keratinocyte cells, pericytes, subtypes of any of the foregoing, cells derived from any of the foregoing, cells derived from induced pluripotent stem cells and mixtures of any of the foregoing. Exemplary cell types include the cell types recited in WO 2017/075631. In some embodiments, the cells are derived from a cell-line shown in Table 2 below.

TABLE 2
Exemplary cell lines

| | | Germ | Commercial |
|---|---|---|---|
| Cell Line | Cell Type | Layer | Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841- | Epithelial (Colon) | Endoderm | ATCC (CRL-1790) |
| CoN | | | |
| HaCat | Keratinocyte | Ectoderm | Addexbio |
| | | | (T0020001) |
| HHSEC | Endothelial (Hepatic | Endoderm | Sciencellonline.com |
| | Sinusoidal) | | (#5000) |
| Huv-EC-C | Endothelial (Embryonic | Mesoderm | ATCC (CRL-1730) |
| | umbilical) | | |
| MCF-10A | Epithelial (Mammary | Ectoderm | ATCC (CRL-10317) |
| | Gland) | | |
| MRC-5 | Fibroblast (Lung) | Mesoderm | ATCC (CCL-171) |
| MSC, human | Mesenchyme (Bone | Mesoderm | ATCC (PCS-500- |
| | Marrow) | | 012) |
| MSC, mouse | Mesenchyme (Bone | Mesoderm | Cyagen |
| | Marrow) | | (MU BMX-01001) |
| WS-1 | Fibroblast (Skin) | Ectoderm | ATCC (CRL-1502) |
| 293F | Epithelial (Embryonic | Mesoderm | Thermo Fisher |
| | Kidney) | | (R790007) |

In some embodiments, the device does not comprise any islet cells or any cells capable of producing insulin in a glucose-response manner. In an embodiment, cells contained in a device of the disclosure, e.g., genetically modified RPE cells, have one or more of the following characteristics: (i) are not capable of producing insulin (e.g., insulin A-chain, insulin B-chain, or proinsulin) in an amount effective to treat diabetes or another disease or condition that may be treated with insulin; (ii) not capable of producing insulin in a glucose-responsive manner; or (iii) not derived from an induced pluripotent stem cell that was engineered or differentiated into insulin-producing pancreatic beta cells.

Cells may be genetically modified to express and secrete immunomodulatory protein(s) using any of a variety of genetic engineering techniques are known in the art. For example, a cell may be transfected with an expression vector comprising an exogenous nucleotide sequence(s) encoding the desired protein(s) operably linked to control elements necessary or useful for gene expression, promoters, ribosomal binding sites, enhancers, poly A signal. Typically, the exogenous sequence encodes a precursor form of the immunomodulatory protein, e.g., includes a secretory signal sequence. In some embodiments, the signal sequence in a precursor immunomodulatory protein consists essentially of an amino acid sequence shown in Table 3 below. In an embodiment, the signal sequence is MGWRAAGALLLALLLHGRLLA (SEQ ID NO:21).

TABLE 3
Exemplary secretory signal peptide sequences

| Source Protein | Amino Acid Sequence |
|---|---|
| Albumin | MKWVTFISLLFLFSSAYS |
| | (SEQ ID NO: 22) |
| Kappa Leader | MVLQTQVFISLLLWISGAYG |
| | (SEQ ID NO: 23) |
| Plasminogen | MQMSPALTCLVLGLALVFGEGSA |
| activator | (SEQ ID NO: 24) |
| inhibitor 1 | |
| Thrombospondin-1 | MGLAWGLGVLFLMHVCGT |
| | (SEQ ID NO: 25) |
| Fibronectin | MLRGPGPGLLLLAVQCLGTAVPSTGASKSKR |
| | (SEQ ID NO: 26) |
| Basement | MGWRAAGALLLALLLHGRLLA |
| membrane- | (SEQ ID NO: 27) |
| specific | |
| heparan sulfate | |
| proteoglycan | |
| core protein | |
| (HSPG2) | |
| Agrin | MAGRSHPGPLRPLLPLLVVAACVLPGAGG |
| | (SEQ ID NO: 28) |
| H7 Leader | MEFGLSWVFLVALFRGVQC |
| | (SEQ ID NO: 29) |
| L2 Leader | MKYLLPTAAAGLLLLAAQPAMA |
| | (SEQ ID NO: 30) |
| HMM34 | MRPTWAWWLFLVLLLALWAPARG |
| | (SEQ ID NO: 31) |
| HMM38 | MWWRLWWLLLLLLLLWPMVWA |
| | (SEQ ID NO: 32) |
| Gaussia | MGVKVLFALICIAVAEA |
| luciferase | (SEQ ID NO: 33) |
| Alpha-1- | MPSSVSWGILLLAGLCCLVPVSLA |
| antitrypsin | (SEQ ID NO: 34) |
| Interleukin-10 | MHSSALLCCLVLLTGVRA |
| | (SEQ ID NO: 35) |

When engineering cells to co-express two or more immunomodulatory proteins, a multicistronic vector may be employed. In some embodiments, a genetically modified cell described herein comprises an exogenous nucleotide sequence, which encodes an immunomodulatory protein, stably inserted into one or more genomic locations (e.g., open chromatin region(s).

In some embodiments, the cells are genetically modified with a regulatable expression system, to allow controlled expression of the immunomodulatory proteins. A variety of such systems are known in the art and include, for example: kill switches (see, e.g., Wu, C. et al., Mol. Ther. Methods Clin Dev. 2014; 1: 14053); On/Off systems (see, e.g., Gossen, M and Gujar, H., Proc. Natl. Acad. Sci. USA, Vol 89, pp. 5547-5551 (1992); Liberles, S., et al., Proc. Natl. Acad. Sci. USA, Vol. 94, pp. 7825-7830 (1997); feed forward and negative feedback systems (Lillacci, G. et al., Nuc. Acids Res, Vol 46, Issue 18 (12 Oct. 2018) pp. 9855-9863); temperature inducible systems (Miller, I. et al., ACS Synth Biol. 2018; 7(4): 1167-1173).

In some embodiments, a genetically modified cell described herein is derived from an ARPE-19 cell, and may express and secrete any of the immunomodulatory proteins described herein (or combinations of such proteins).

In an embodiment, a genetically modified cell described herein (e.g., derived from an ARPE-19 cell) comprises any of the coding sequences described herein, e.g., any of the coding sequences shown in FIGS. 1-3.

### Features of Devices

A device of the present disclosure comprises at least one barrier that prevents immune cells from contacting cells contained inside the device. At least a portion of the barrier needs to be sufficiently porous to allow proteins (e.g., immunomodulatory protein) expressed and secreted by the cells to exit the device. A variety of device configurations known in the art are suitable.

The device (e.g., particle) can have any configuration and shape appropriate for supporting the viability and productivity of the contained cells after implant into the intended target location. As non-limiting examples, device shapes may be cylinders, rectangles, disks, ovoids, stellates, or spherical. The device can be comprised of a mesh-like or nested structure. In some embodiments, a device is capable of preventing materials over a certain size from passing through a pore or opening. In some embodiments, a device (e.g., particle) is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

In an embodiment, the device is a macroencapsulation device. Nonlimiting examples of macrodevices are described in: WO 2019/068059, WO 2019/169089, U.S. Pat. Nos. 9,526,880, 9,724,430 and 8,278,106; European Patent No. EP742818B1, and Sang, S. and Roy, S., Biotechnol. Bioeng. 113(7):1381-1402 (2016).

In an embodiment, the device is a macrodevice having one or more cell-containing compartments. A device with two or more cell-containing compartments may be configured to produce two or more immunomodulatory proteins, e.g., cells expressing a first immunomodulatory protein would be placed in one compartment and cells expressing a second immunomodulatory protein would be placed in a separate compartment. WO 2018/232027 describes a device with multiple cell-containing compartments formed in a micro-fabricated body and covered by a porous membrane.

In an embodiment, the device is configured as a thin, flexible strand as described in U.S. Pat. No. 10,493,107. This strand comprises a substrate, an inner polymeric coating surrounding the substrate and an outer hydrogel coating surrounding the inner polymeric coating. The protein-expressing cells are positioned in the outer coating.

In some embodiments, a device (e.g., particle) has a largest linear dimension (LLD), e.g., mean diameter, or size that is at least about 0.5 millimeter (mm), preferably about 1.0 mm, about 1.5 mm or greater. In some embodiments, a device can be as large as 10 mm in diameter or size. For example, a device or particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm.

In some embodiments, a device of the disclosure (e.g., particle, capsule) comprises at least one pore or opening, e.g., to allow for the free flow of materials. In some embodiments, the mean pore size of a device is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of a device is between about 0.1 μm to 10 μm. In some embodiments, the mean pore size of a device is between about 0.1 μm to 5 μm. In some embodiments, the mean pore size of a device is between about 0.1 μm to 1 μm.

In some embodiments, the device comprises a semi-permeable, biocompatible membrane surrounding the genetically modified cells that are encapsulated in a polymer composition (e.g., an alginate hydrogel). The membrane pore size is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

In an embodiment, the device may contain a cell-containing compartment that is surrounded with a barrier compartment formed from a cell-free biocompatible material, such as the core-shell microcapsules described in Ma, M et al., Adv. Healthc Mater., 2(5):667-672 (2012). Such a barrier compartment could be used with or without the semi-permeable membrane.

Cells in the cell-containing compartment(s) of a device of the disclosure may be encapsulated in a polymer composition. The polymer composition may comprise one or more hydrogel-forming polymers. In addition to the polymer composition in the cell-containing compartment(s), the device (e.g., macrodevice, particle, hydrogel capsule) may comprise or be formed from materials such as metals, metallic alloys, ceramics, polymers, fibers, inert materials, and combinations thereof. A device may be completely made up of one type of material, or may comprise other materials within the cell-containing compartment and any other compartments.

In some embodiments, the device comprises a metal or a metallic alloy. In an embodiment, one or more of the compartments in the device (e.g., the first compartment, the second compartment, or all compartments) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing devices, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device comprises a ceramic. In an embodiment, one or more of the compartments in the device (e.g., the first compartment, the second compartment, or all compartments) comprises a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing devices, the amount of ceramic (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment. In such embodiments, the thickness of the second (outer) compartment means the average distance between the outer boundary of the second compartment and the interface between the two compartments, e.g., the average of the distances measured at each of the thinnest and thickest points visually observed in the outer compartment. In some embodiments (e.g., the device is about 1.5 mm in diameter), the thinnest and thickest distances for the outer compartment are between 25 and 110 micrometers (μm) and between 270 and 480 μm, respectively. In some embodiments, the thickness of the outer compartment is greater than about 10 nanometers (nm), preferably 100 nm or greater and can be as large as 1 millimeter (mm). For example, the thickness (e.g., average distance) of the outer compartment in a hydrogel capsule device described herein may be 10 nm to 1 mm, 100 nm to 1 mm, 500 nm to 1 millimeter, 1 micrometer (μm) to 1 mm, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness (e.g., average distance) of the outer compartment is 100 nm to 1 mm, between 1 μm and 1 mm, between 1 μm and 500 μm or between 5 μm and 1 mm. In some embodiments, the thickness (e.g., average distance) of the outer compartment is between about 50 μm and about 100 μm. In some embodiments (e.g., the device is about 1.5 mm in diameter), the thickness of the outer compartment (e.g., average distance) is between about 180 μm and 260 μm or between about 310 μm and 440 μm.

In some embodiments of a two-compartment hydrogel capsule device, the mean pore size of the cell-containing inner compartment and the outer compartment is substantially the same. In some embodiments, the mean pore size of the inner compartment and the second compartment differ by about 1.5%, 2%, 5%, 7.5%, 10%, 15%, 20%, 25%, 30%, 35%, 40%, 45%, 50%, 55%, 60%, 65%, 70%, 75%, or more. In some embodiments, the mean pore size of the device (e.g., mean pore size of the first compartment and/or mean pore size of the second compartment) is dependent on a number of factors, such as the material(s) within each compartment and the presence and density of a compound of Formula (I).

The device may form part of a plurality of substantially the same devices in a preparation (e.g., composition). In some embodiments, the devices (e.g., particles, hydrogel capsules) in the preparation have a mean diameter or size between about 0.5 mm to about 8 mm. In some embodiments, the mean diameter or size of devices in the preparation is between about 0.5 mm to about 4 mm or between about 0.5 mm to about 2 mm. In some embodiments, the devices in the preparation are two-compartment hydrogel capsules and have a mean diameter or size of about 0.7 mm to about 1.3 mm or about 1.2 mm to about 1.8 mm.

In some embodiments, the surface of the device comprises a compound capable of mitigating the FBR, an afibrotic compound as described herein below. For devices comprising a barrier compartment surrounding the cell-containing compartment, the afibrotic compound may covalently modify a polymer disposed throughout the barrier compartment and optionally throughout the cell-containing compartment.

In some embodiments, one or more compartments in a device comprises an afibrotic polymer, e.g., an afibrotic compound of Formula (I) covalently attached to a polymer. In an embodiment, some or all the monomers in the afibrotic polymer are modified with the same compound of Formula (I). In some embodiments, some or all the monomers in the afibrotic polymer are modified with different compounds of Formula (I). In some embodiments in which the device is a 2-compartment hydrogel capsule, the afibrotic polymer is present only in the outer, barrier compartment.

One or more compartments in a device may comprise an unmodified polymer that is the same or different than the polymer in any afibrotic polymer that is present in the device. In an embodiment, the first compartment, second compartment or all compartments in the device comprise the unmodified polymer.

Each of the modified and unmodified polymers in the device may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof. In polymer-containing devices, the amount of a polymer (e.g., by % weight of the device, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, one or more of the modified and unmodified polymers in the device comprises a polyethylene. Exemplary polyethylenes include ultra-low-density polyethylene (ULDPE) (e.g., with polymers with densities ranging from 0.890 to 0.905 g/cm3, containing comonomer); very-low-density polyethylene (VLDPE) (e.g., with polymers with densities ranging from 0.905 to 0.915 g/cm3, containing comonomer); linear low-density polyethylene (LLDPE) (e.g., with polymers with densities ranging from 0.915 to 0.935 g/cm3, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm3, may or may not contain comonomer); high-density polyethylene (HDPE) (e.g., with polymers with densities ranging from 0.940 to 0.970 g/cm3, may or may not contain comonomer) and polyethylene glycol.

In some embodiments, one or more of the modified and unmodified polymers in the device comprises a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3-Plastics Used in Medical Devices, (2014):21-53.

In some embodiments, one or more of the modified and unmodified polymers in the device comprises a polypropylene. Exemplary polystyrenes include general purpose or crystal (PS or GPPS), high impact (HIPS), and syndiotactic (SPS) polystyrene.

In some embodiments, one or more of the modified and unmodified polymers comprises a comprises a thermoplastic elastomer (TPE). Exemplary TPEs include (i) TPA-polyamide TPE, comprising a block copolymer of alternating hard and soft segments with amide chemical linkages in the hard blocks and ether and/or ester linkages in the soft blocks; (ii) TPC-co-polyester TPE, consisting of a block copolymer of alternating hard segments and soft segments, the chemical linkages in the main chain being ester and/or ether; (iii) TPO-olefinic TPE, consisting of a blend of a polyolefin and a conventional rubber, the rubber phase in the blend having little or no cross-linking; (iv) TPS-styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU-urethane TPE, consisting of a block copolymer of alternating hard and soft segments with urethane chemical linkages in the hard blocks and ether, ester or carbonate linkages or mixtures of them in the soft blocks; (vi) TPV—thermoplastic rubber vulcanizate consisting of a blend of a thermoplastic material and a conventional rubber in which the rubber has been cross-linked by the process of dynamic vulcanization during the blending and mixing step; and (vii) TPZ-unclassified TPE comprising any composition or structure other than those grouped in TPA, TPC, TPO, TPS, TPU, and TPV.

In some embodiments, the unmodified polymer is an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In an embodiment, the unmodified alginate has a molecular weight of 150 kDa-250 kDa and a G:M ratio of ≥1.5.

In some embodiments, the afibrotic polymer comprises an alginate chemically modified with a Compound of Formula (I) The alginate in the afibrotic polymer may be the same or different than any unmodified alginate that is present in the device. In an embodiment, the density of the Compound of Formula (I) in the afibrotic alginate (e.g., amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (I) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described in WO2020069429), and in certain embodiments, the density of a Compound of Formula (I) (e.g., Compound 101) is between about 1.0% w/w and about 3.0% w/w, between about 1.3% w/w and about 2.5% w/w or between about 1.5% w/w and 2.2% w/w.

In alginate-containing devices, the amount of modified and unmodified alginates (e.g., by % weight of the device, actual weight of the alginate) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate.

In an embodiment, the device comprises at least one cell-containing compartment, and in some embodiments contains two, three, four or more cell-containing compartments. In an embodiment, each cell-containing compartment comprises a plurality of cells (e.g., live cells) and the cells in at least one of the compartments are capable of expressing and secreting at least one immunomodulatory protein when the device is implanted into a subject. In some embodiments, the cells in a single cell-containing compartment express two or more immunomodulatory proteins, e.g., proteins with complementary activities useful for treating a particular IMID.

In an embodiment, all the cells in a cell-containing compartment are derived from a single parental cell-type or a mixture of at least two different parental cell types. In an embodiment, all of the cells in a cell-containing compartment are derived from the same parental cell type, but a first plurality of the derived cells are genetically modified to express a first immunomodulatory protein, and a second plurality of the derived cells are genetically modified to express a second immunomodulatory protein. In devices with two or more cell-containing compartments, the cells and the immunomodulatory protein(s) produced thereby may be the same or different in each cell-containing compartment. In some embodiments, all of the cell-containing compartments are surrounded by a single barrier compartment. In some embodiments, the barrier compartment is substantially cell-free.

In an embodiment, cells to be incorporated into a device described herein, e.g., a hydrogel capsule, are prepared in the form of a cell suspension prior to being encapsulated within the device. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In addition to immunomodulatory protein(s) expressed by the encapsulated cells, a device (e.g., capsule, particle) may comprise one or more exogenous agents that are not expressed by the cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), an active or inactive fragment of a protein or polypeptide, a small molecule, or drug. In an embodiment, the device is configured to release such an exogenous agent.

### Afibrotic (e.g., FBR-mitigating) Compounds

In some embodiments, the devices described herein comprise at least one compound of Formula (I):

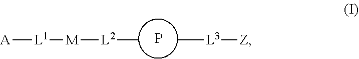

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D}), —NCN—, —C(═N(R^{C})(R^{D}))O—, —S—, —S(O)ₓ—, —OS(O)ₓ—, —N(R^{C})S(O)ₓ—, —S(O)\N(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶,
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷; each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, the compound of Formula (I) is a compound of Formula (I-a):

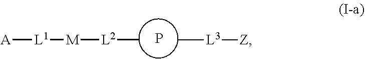

or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C}), —N(R^{C})N(R^{D}), N(R^{C})C(O)(C₁-C₆-alkylene)-, —N(R^{C})C(O)(C₁-C₆-alkenylene)-, —NCN—, —C(═N(R^{C})(R^{D}))O—, —S—, —S(O)—, —OS(O)—, —N(R^{C})S(O)—, —S(O)ₓN(R^{C})—, —P(R^{F})_{y}—, —Si(OR^{A})₂-, —Si(R^{G})(OR^{A})—, —B(OR^{A}), or a metal, each of which is optionally linked to an attachment group (e.g., an attachment group described herein) and optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is heteroaryl optionally substituted by one or more R⁴;
Z is alkyl, alkenyl, alkynyl, heteroalkyl, cycloalkyl, heterocyclyl, aryl, or heteroaryl, each of which is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

In some embodiments, for Formulas (I) and (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)—. In some embodiments, A is —N(R^{C})—. In some embodiments, A is —N(R^{C})—, and R^{C} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) and (I-a), L¹ is a bond, alkyl, or heteroalkyl. In some embodiments, L¹ is a bond or alkyl. In some embodiments, L¹ is a bond. In some embodiments, L′ is alkyl. In some embodiments, L¹ is C₁-C₆ alkyl. In some embodiments, L′ is —CH₂—, —CH(CH₃), —CH₂CH₂CH₂, or —CH₂CH₂—. In some embodiments, L³ is —CH₂CH₂—. In some embodiments, L′ is —CH₂— or —CH₂CH₂—.

In some embodiments, for Formulas (I) and (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) and (I-a), M is absent, alkyl, heteroalkyl, aryl, or heteroaryl. In some embodiments, for Formulas (I) and (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂—)_{z}, wherein z is an integer selected from 1 to 10. In some embodiments, z is an integer selected from 1 is to 5. In some embodiments, M is —(OCH₂)₂—, (—OCH₂CH₂—)₂, (—OCH₂CH₂—)₃, (—OCH₂CH₂—)₄, or (—OCH₂CH_{2 5}. In some embodiments, M is —OCH₂CH₂—, (—OCH₂CH₂—)₂, (—OCH₂CH₂—)₃, or (—OCH₂CH₂—)₄. In some embodiments, M is (—OCH₂—)₃. In some embodiments, M is aryl. In some embodiments, M is phenyl. In some embodiments, M is unsubstituted phenyl. In some embodiments, M is

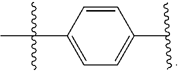 [SMILES: *c1ccc(*)cc1]

In some embodiments, M is

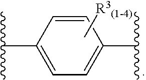

In some embodiments, M is phenyl substituted with 1-4 R³ (e.g., 1 R³). In some embodiments, R³ is CF₃.

In some embodiments, for Formulas (I) and (I-a), P is absent, heterocyclyl, or heteroaryl. In some embodiments, for Formulas (I) and (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, or pyrrolyl. In some embodiments, P is imidazolyl. In some embodiments, P is 1,2,3-triazolyl. In some embodiments, P is

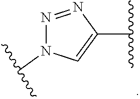 [SMILES: *c1cn(*)nn1]

In some embodiments, P is

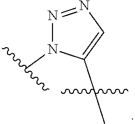 [SMILES: *c1cnnn1*]

In some embodiments, P is

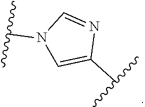 [SMILES: *c1cn(*)cn1]

In some embodiments, P is heterocyclyl. In some embodiments, P is heterocyclyl. In some embodiments, P is a 5-membered heterocyclyl. In some embodiments, P is imidazolidinonyl. In some embodiments, P is

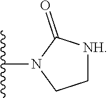 [SMILES: *N1CCNC1=O]

In some embodiments, P is thiomorpholinyl-1,1-dioxidyl. In some embodiments, P is

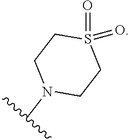 [SMILES: *N1CCS(=O)(=O)CC1]

In some embodiments, for Formulas (I) and (I-a), Z is alkyl, heteroalkyl, cycloalkyl, heterocyclyl, aryl, or heteroaryl. In some embodiments, for Formulas (I) and (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl, 5-membered heterocyclyl, or 6-membered heterocyclyl. In some embodiments, Z is a 6-membered oxygen-containing heterocyclyl. In some embodiments, Z is tetrahydropyranyl. In some embodiments, Z is

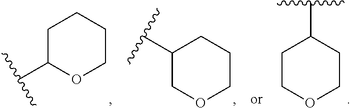

In some embodiments, Z is a 4-membered oxygen-containing heterocyclyl. In some embodiments, Z is

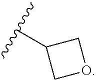 [SMILES: *C1COC1]

In some embodiments, Z is a bicyclic oxygen-containing heterocyclyl. In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. In some embodiments, Z is a 6-membered heterocyclyl containing a nitrogen atom and a sulfur atom. In some embodiments, Z is thiomorpholinyl-1,1-dioxidyl. In some embodiments, Z is

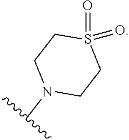 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

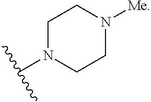 [SMILES: *N1CCN(C)CC1]

In some embodiments, Z is a bicyclic heterocyclyl. In some embodiments, Z is a bicyclic heterocyclyl In some embodiments, Z is a bicyclic nitrogen-containing heterocyclyl, optionally substituted with one or more R⁵. In some embodiments, Z is 2-oxa-7-azaspiro[3.5]nonanyl In some embodiments, Z is

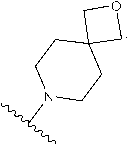 [SMILES: *N1CCC2(CC1)COC2]

In some embodiments, Z is 1-oxa-3,8-diazaspiro[4.5]decan-2-one. In some embodiments, Z is

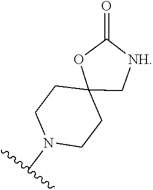 [SMILES: *N1CCC2(CC1)CNC(=O)O2]

In some embodiments, for Formulas (I) and (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) and (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) and (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

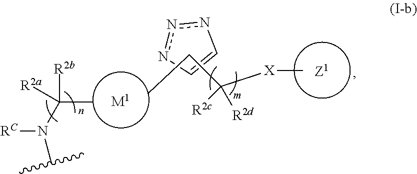

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SREI, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O) [symbol] , —C(O)N(R^{C1}), cycloalkyl, heterocyclyl or heteroaryl; each R^{A1}, R^{B1}, RCIDI, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

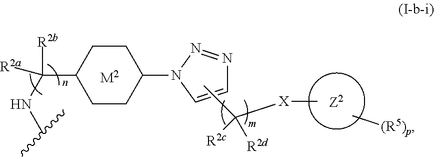

or a pharmaceutically acceptable salt thereof, wherein Ring M² is aryl or heteroaryl optionally substituted with one or more R³; Ring Z² is cycloalkyl, heterocyclyl, aryl or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} [symbol] is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

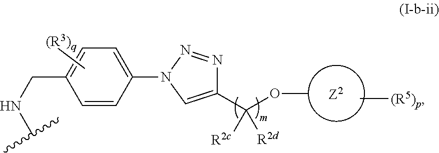

or a pharmaceutically acceptable salt thereof, wherein Ring Z² is cycloalkyl, heterocyclyl, aryl or heteroaryl; [symbol] each of R^{2c} and R^{2d} is independently hydrogen, alkyl, or heteroalkyl, or R^{2c} and R and taken together to form an oxo group; [symbol] leach R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} [symbol] and R^{B1} is independently hydrogen, alkyl, or heteroalkyl; m is 1, 2, 3, 4, 5, or 6; p is 0, 1, 2, 3, 4, 5, or 6; q is 0, 1, 2, 3, or 4; and “ [symbol] ” refers to a connection to an attachment group or a polymer described herein.

In some embodiments, the compound of Formula (I) is a compound of Formula (I-c):

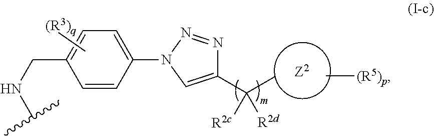

or a pharmaceutically acceptable salt thereof, wherein Ring Z² is cycloalkyl, heterocyclyl aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR, —C(O)OR, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

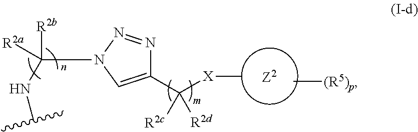

or a pharmaceutically acceptable salt thereof, wherein Ring Z² is cycloalkyl, heterocyclyl aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

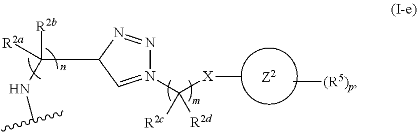

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

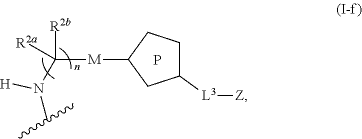

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, OR^{A1}, —C(O)OR^{A1}, or —C(O)R^{B1}; each R^{A1} and R^{B1} is independently hydrogen, alkyl, or heteroalkyl; n is 1, 2, 3, 4, 5, or 6; and “ [symbol] ” refers to a connection to an attachment group or a polymer described herein.

In some embodiments, the compound of Formula (I) is a compound of Formula (II):

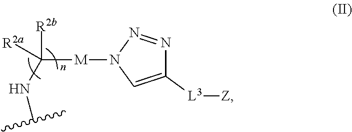

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR⁵, wherein alkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

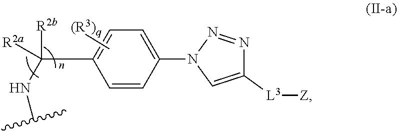

or a pharmaceutically acceptable salt thereof, wherein L³ is alkyl or heteroalkyl, each of which is optionally substituted with one or more R² [symbol] Z is hydrogen, alkyl, heteroalkyl or —OR^{A}, heteroalkyl are optionally substituted with one or more R⁵; each of R^{2a} and R^{2b} is independently hydrogen, alkyl, or heteroalkyl, or R^{2a} and R^{2b} is taken together to form an oxo group each R², R³, and R⁵ is independently heteroalkyl, halogen, oxo, —OR^{A1}, —C(O)OR^{A1B1}; R^{A} is hydrogen, alkyl, or heteralkyl; each R^{A1} and R^{B1} is independently hydrogen, alkyl, or heteroalkyl; n is 1, 2, 3, 4, 5, or 6; and “ [symbol] ” refers to a connection to an attachment group or a polymer described herein.

In some embodiments, the compound of Formula (I) is a compound of Formula (III):

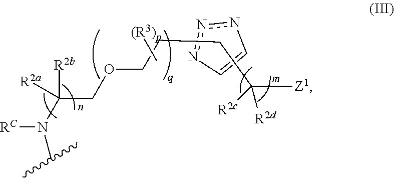

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b}, R^{2c} and R^{2d} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

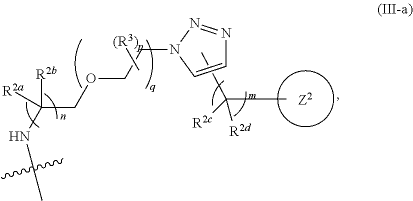

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}. —C(O)OR^{A1}, or —C(O)R^{B1}; each R^{A1} and R^{B1} is independently hydrogen, alkyl, or heteroalkyl; m and [symbol] are each independently 1, 2, 3, 4, 5, or 6; p is 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

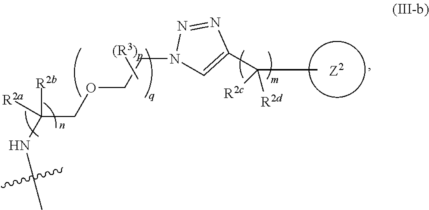

or a pharmaceutically acceptable salt thereof, wherein Ring Z² is cycloalkyl, heterocyclyl, aryl, or heteroaryl each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m is 0, 1, 2, 3, 4, 5, or 6; n is 1, 2, 3, 4, 5, or 6; p is 0, 1, 2, 3, or 4; q is an integer from 0 to 25; and “ [symbol] ” refers to a connection to an attachment group or a polymer described herein.

In some embodiments, the compound of Formula (III-a) is a compound of Formula (III-c):

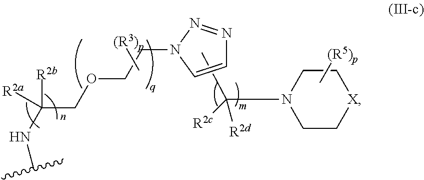

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

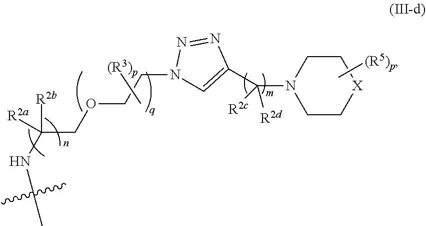

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4; q is an integer from 0 to 25; x is 0, 1, or 2; and “ [symbol] ” refers to a connection to an attachment group or a polymer described herein.

In some embodiments, the compound of Formula (I) is a compound of Formula (III-e):

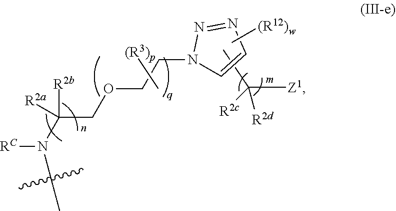

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; w is 0 or 1; and “ [symbol] ” refers to a connection to an attachment group or a polymer described herein.

In some embodiments, the compound of Formula (I) is a compound of Formula (III-f):

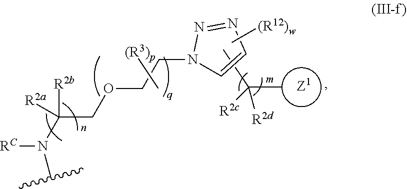

or a pharmaceutically acceptable salt thereof, wherein Ring Z¹ is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; w is 0 or 1; and “ [symbol] ” refers to a connection to an attachment group or a polymer described herein.

In some embodiments, the compound of Formula (I) is a compound of Formula (III-g):

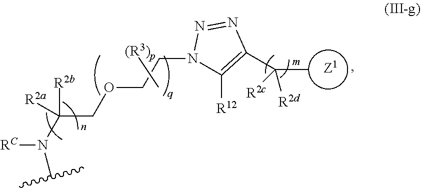

or a pharmaceutically acceptable salt thereof, wherein Ring Z¹ is cycloalkyl, heterocyclyl, aryl, or heteroaryl, each of which is optionally substituted with 1-5 R⁵; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; and “ [symbol] ” refers to a connection to an attachment group or a polymer described herein.

In some embodiments, the compound of Formula (I) is a compound of Formula (III-h):

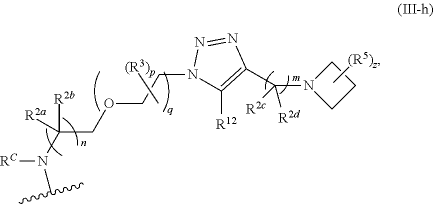

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound of Formula (I) is a compound of Formula (III-i):

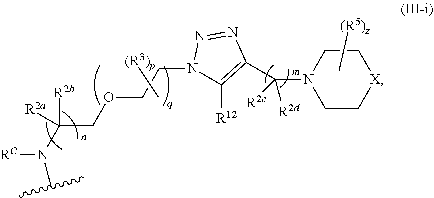

or a pharmaceutically acceptable salt thereof, wherein X is C(R′)(R″), N(R′), or S(O)ₓ; each of R′ and R″ is independently hydrogen, alkyl, or halogen; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹² is halo (e.g., C₁).

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L′ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b). In some embodiments of Formula (I-b), each of R^{2a} and R^{2b} is independently hydrogen or CH₃, each of R^{2c} and R^{2d} is independently hydrogen, m is 1 or 2, n is 1, X is O, M¹ is phenyl optionally substituted with one or more R³, R³ is —CF₃, and Z¹ is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 108, Compound 109, or Compound 111.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is O, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R²⁶, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-e). In some embodiments of Formula (I-e), each of R^{2a}, R^{2b}, R^{2c}, and R^{2d} is independently hydrogen, n is 1, m is 2, X is O, and Z² is heterocyclyl (e.g., an oxygen-containing heterocyclyl, e.g., tetrahydropyranyl). In some embodiments, the compound of Formula (I-e) is Compound 107.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0,
n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-a). In some embodiments of Formula (III-a), each of R^{2a}, R^{2b}, R^{2c}, R^{2d}, and R^{C} is independently hydrogen, m is 1, n is 2, q is 4, p is 0, w is 0, and Z¹ is heterocyclyl (e.g., a nitrogen-containing heterocyclyl, e.g., thiomorpholinyl-1,1-dioxide). In some embodiments, the compound of Formula (III-a) is Compound 119.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl). In some embodiments, the compound of Formula (III-b) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (III-c). In some embodiments of Formula (III-c), each of R^{2a}, R^{2b}, R^{2c}, R^{2d}, R^{C} and R¹² is independently hydrogen, m is 1, n is 2, q is 3, p is 0, z is 1, and R⁵ is (e.g., —S(O)₂(CH₃)). In some embodiments, the compound of Formula (III-c) is Compound 123.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 4, or a pharmaceutically acceptable salt thereof. In some embodiments, the exterior surface and/or one or more compartments within a device described herein comprises a small molecule compound shown in Table 4, or a pharmaceutically acceptable salt thereof.

TABLE 4
Exemplary afibrotic (FBR-mitigating) compounds

| Compound No. | Structure |
|---|---|
| 100 | 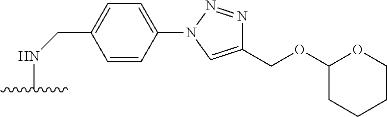 [SMILES: *NCc1ccc(-n2cc(COC3CCCCO3)nn2)cc1] |
| 101 | 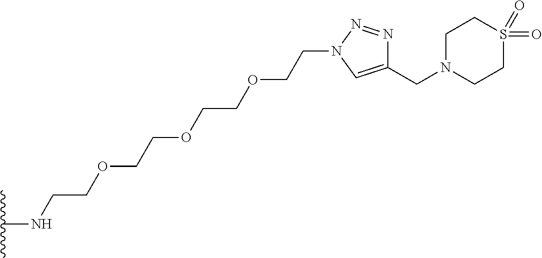 [SMILES: *NCCOCCOCCOCCn1cc(CN2CCS(=O)(=O)CC2)nn1] |
| 102 | 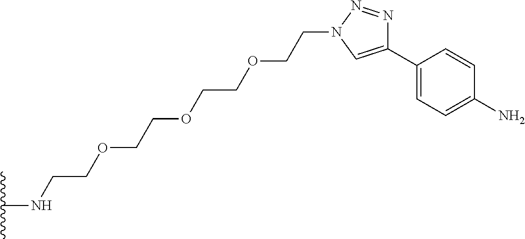 [SMILES: *NCCOCCOCCOCCn1cc(-c2ccc(N)cc2)nn1] |
| 103 | 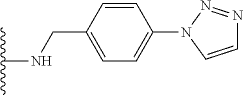 [SMILES: *NCc1ccc(-n2ccnn2)cc1] |
| 104 | 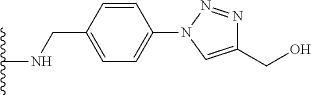 [SMILES: *NCc1ccc(-n2cc(CO)nn2)cc1] |
| 105 | 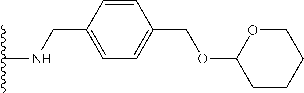 [SMILES: *NCc1ccc(COC2CCCCO2)cc1] |
| 106 | 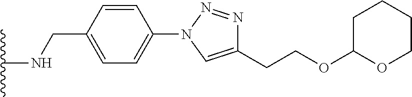 [SMILES: *NCc1ccc(-n2cc(CCOC3CCCCO3)nn2)cc1] |
| 107 | 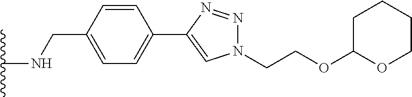 [SMILES: *NCc1ccc(-c2cn(CCOC3CCCCO3)nn2)cc1] |
| 108 | 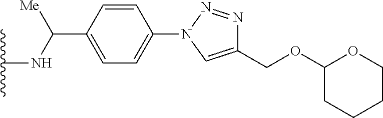 [SMILES: *NC(C)c1ccc(-n2cc(COC3CCCCO3)nn2)cc1] |
| 109 | 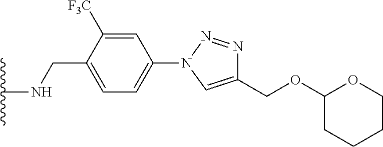 [SMILES: *NCc1ccc(-n2cc(COC3CCCCO3)nn2)cc1C(F)(F)F] |
| 110 | 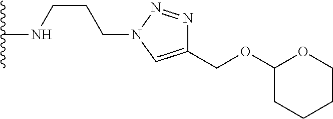 [SMILES: *NCCCn1cc(COC2CCCCO2)nn1] |
| 111 | 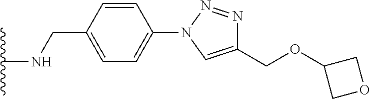 [SMILES: *NCc1ccc(-n2cc(COC3COC3)nn2)cc1] |
| 112 | 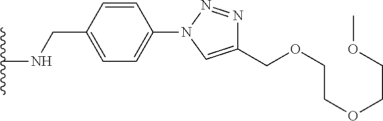 [SMILES: *NCc1ccc(-n2cc(COCCOCCOC)nn2)cc1] |
| 113 | 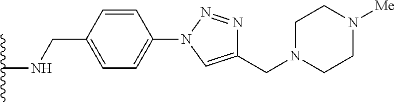 [SMILES: *NCc1ccc(-n2cc(CN3CCN(C)CC3)nn2)cc1] |
| 114 | 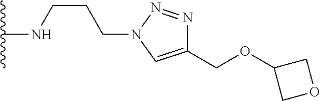 [SMILES: *NCCCn1cc(COC2COC2)nn1] |
| 115 | 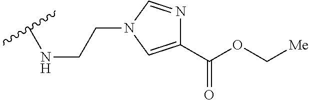 [SMILES: *NCCn1cnc(C(=O)OCC)c1] |
| 116 | 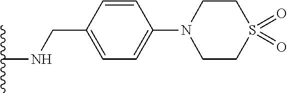 [SMILES: *NCc1ccc(N2CCS(=O)(=O)CC2)cc1] |
| 117 | 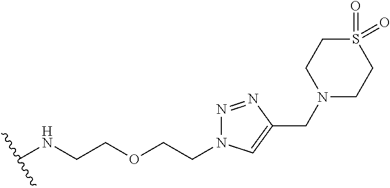 [SMILES: *NCCOCCn1cc(CN2CCS(=O)(=O)CC2)nn1] |
| 118 | 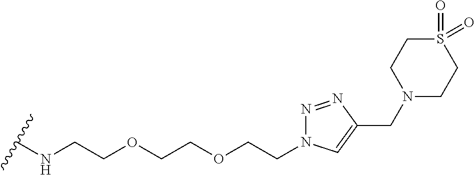 [SMILES: *NCCOCCOCCn1cc(CN2CCS(=O)(=O)CC2)nn1] |
| 119 | 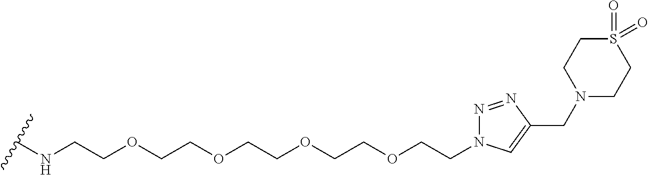 [SMILES: *NCCOCCOCCOCCOCCn1cc(CN2CCS(=O)(=O)CC2)nn1] |
| 120 | 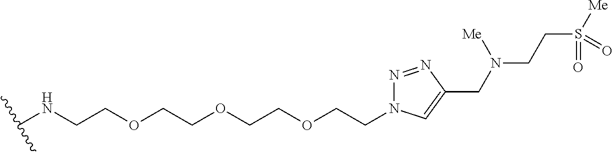 [SMILES: *NCCOCCOCCOCCn1cc(CN(C)CCS(C)(=O)=O)nn1] |
| 121 | 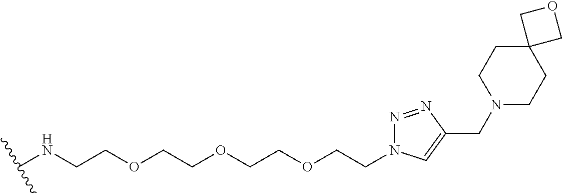 [SMILES: *NCCOCCOCCOCCn1cc(CN2CCC3(CC2)COC3)nn1] |
| 122 | 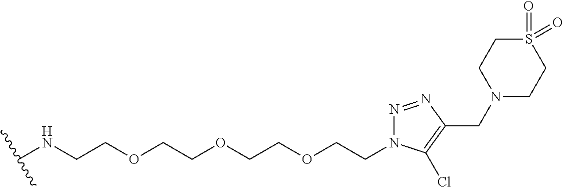 [SMILES: *NCCOCCOCCOCCn1nnc(CN2CCS(=O)(=O)CC2)c1Cl] |
| 123 | 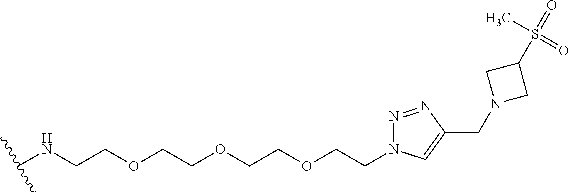 [SMILES: *NCCOCCOCCOCCn1cc(CN2CC(S(C)(=O)=O)C2)nn1] |
| 124 | 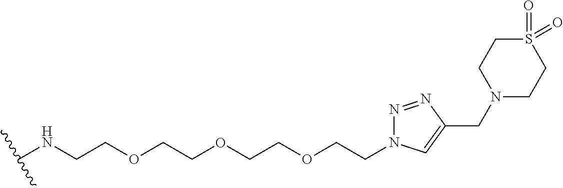 [SMILES: *NCCOCCOCCOCCn1cc(CN2CCS(=O)(=O)CC2)nn1] |
| 125 | 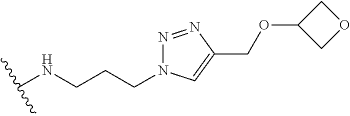 [SMILES: *NCCCn1cc(COC2COC2)nn1] |
| 126 | 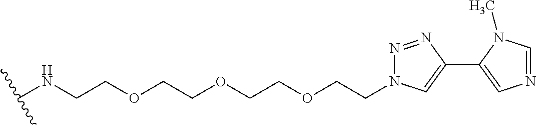 [SMILES: *NCCOCCOCCOCCn1cc(-c2cncn2C)nn1] |
| 127 | 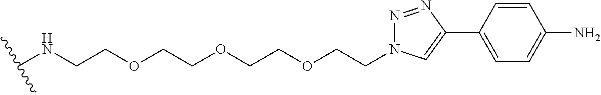 [SMILES: *NCCOCCOCCOCCn1cc(-c2ccc(N)cc2)nn1] |
| 128 | 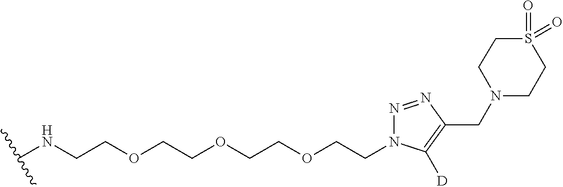 [SMILES: *NCCOCCOCCOCCn1nnc(CN2CCS(=O)(=O)CC2)c1[2H]] |
| 129 | 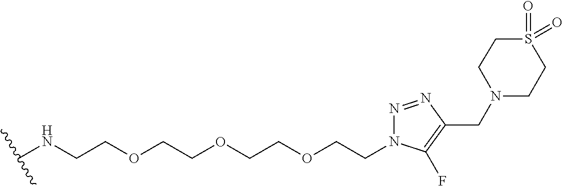 [SMILES: *NCCOCCOCCOCCn1nnc(CN2CCS(=O)(=O)CC2)c1F] |
| 130 | 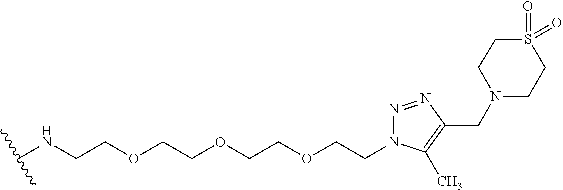 [SMILES: *NCCOCCOCCOCCn1nnc(CN2CCS(=O)(=O)CC2)c1C] |
| 131 | 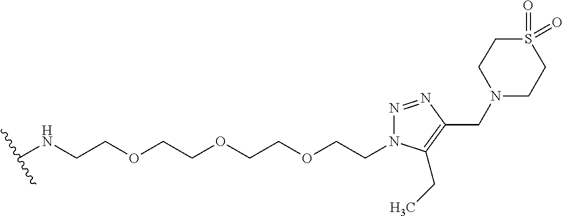 [SMILES: *NCCOCCOCCOCCn1nnc(CN2CCS(=O)(=O)CC2)c1CC] |
| 132 | 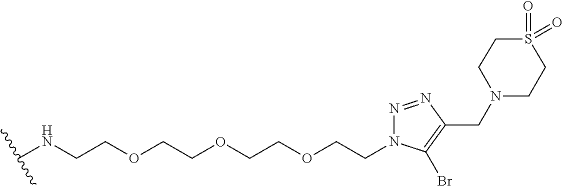 [SMILES: *NCCOCCOCCOCCn1nnc(CN2CCS(=O)(=O)CC2)c1Br] |
| 133 | 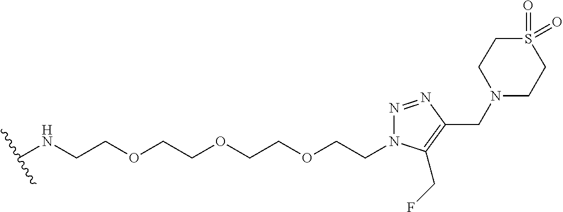 [SMILES: *NCCOCCOCCOCCn1nnc(CN2CCS(=O)(=O)CC2)c1CF] |
| 134 | 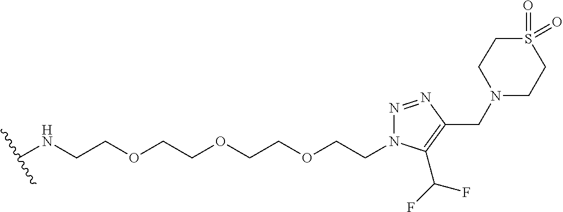 [SMILES: *NCCOCCOCCOCCn1nnc(CN2CCS(=O)(=O)CC2)c1C(F)F] |
| 135 | 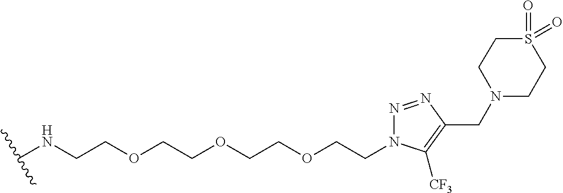 [SMILES: *NCCOCCOCCOCCn1nnc(CN2CCS(=O)(=O)CC2)c1C(F)(F)F] |
| 136 | 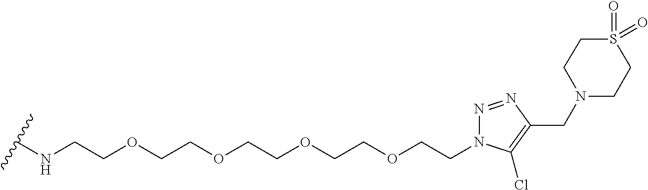 [SMILES: *NCCOCCOCCOCCOCCn1nnc(CN2CCS(=O)(=O)CC2)c1Cl] |
| 137 | 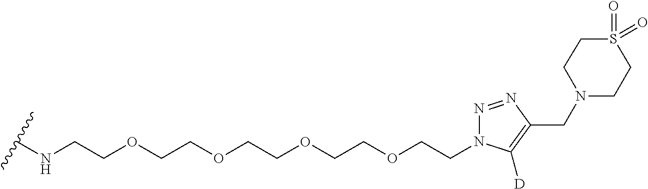 [SMILES: *NCCOCCOCCOCCOCCn1nnc(CN2CCS(=O)(=O)CC2)c1[2H]] |
| 138 | 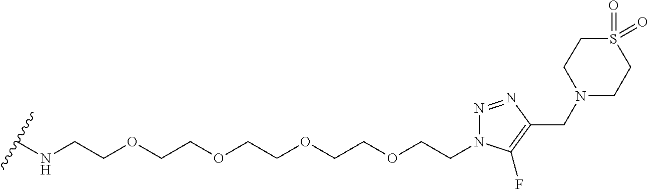 [SMILES: *NCCOCCOCCOCCOCCn1nnc(CN2CCS(=O)(=O)CC2)c1F] |
| 139 | 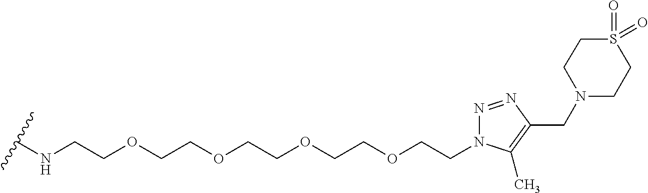 [SMILES: *NCCOCCOCCOCCOCCn1nnc(CN2CCS(=O)(=O)CC2)c1C] |
| 140 | 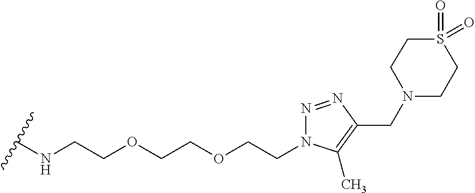 [SMILES: *NCCOCCOCCn1nnc(CN2CCS(=O)(=O)CC2)c1C] |
| 141 | 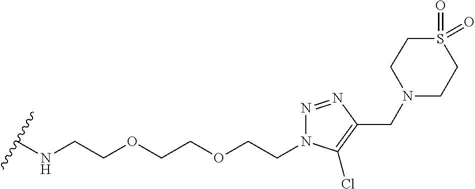 [SMILES: *NCCOCCOCCn1nnc(CN2CCS(=O)(=O)CC2)c1Cl] |
| 142 | 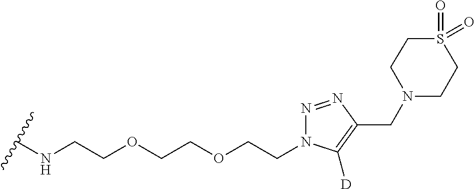 [SMILES: *NCCOCCOCCn1nnc(CN2CCS(=O)(=O)CC2)c1[2H]] |
| 143 | 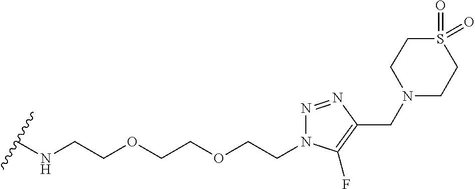 [SMILES: *NCCOCCOCCn1nnc(CN2CCS(=O)(=O)CC2)c1F] |
| 144 | 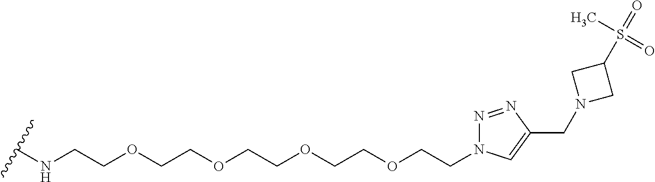 [SMILES: *NCCOCCOCCOCCOCCn1cc(CN2CC(S(C)(=O)=O)C2)nn1] |
| 145 | 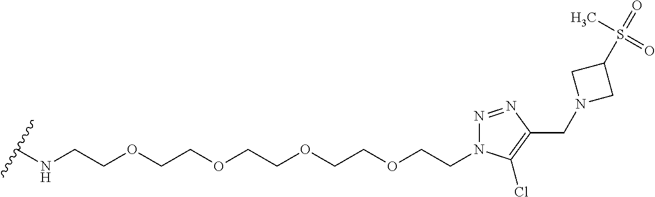 [SMILES: *NCCOCCOCCOCCOCCn1nnc(CN2CC(S(C)(=O)=O)C2)c1Cl] |
| 146 | 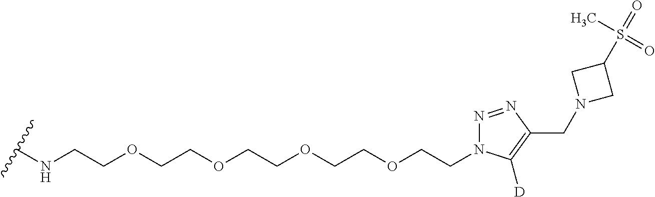 [SMILES: *NCCOCCOCCOCCOCCn1nnc(CN2CC(S(C)(=O)=O)C2)c1[2H]] |
| 147 | 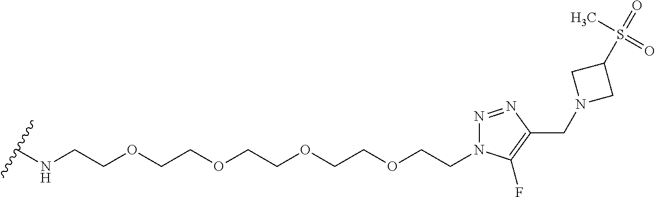 [SMILES: *NCCOCCOCCOCCOCCn1nnc(CN2CC(S(C)(=O)=O)C2)c1F] |
| 148 | 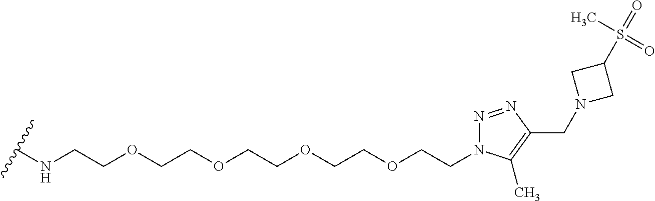 [SMILES: *NCCOCCOCCOCCOCCn1nnc(CN2CC(S(C)(=O)=O)C2)c1C] |
| 149 | 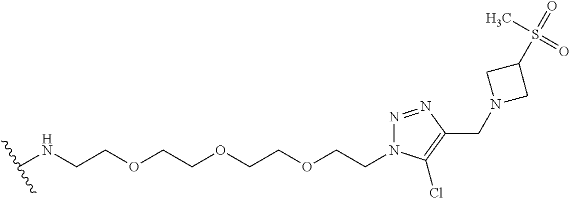 [SMILES: *NCCOCCOCCOCCn1nnc(CN2CC(S(C)(=O)=O)C2)c1Cl] |
| 150 | 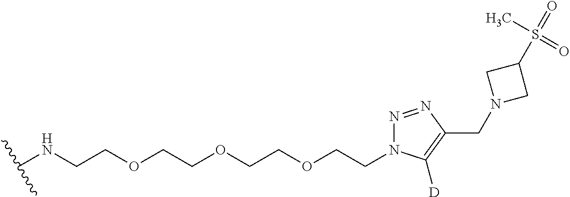 [SMILES: *NCCOCCOCCOCCn1nnc(CN2CC(S(C)(=O)=O)C2)c1[2H]] |
| 151 | 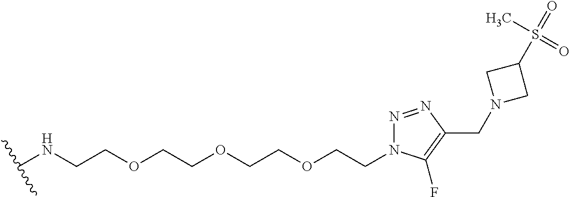 [SMILES: *NCCOCCOCCOCCn1nnc(CN2CC(S(C)(=O)=O)C2)c1F] |
| 152 | 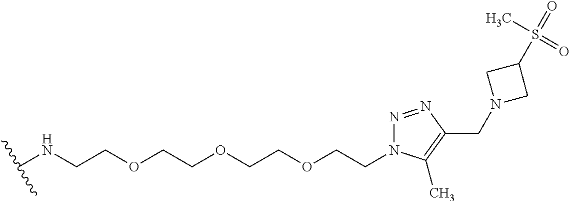 [SMILES: *NCCOCCOCCOCCn1nnc(CN2CC(S(C)(=O)=O)C2)c1C] |
| 153 | 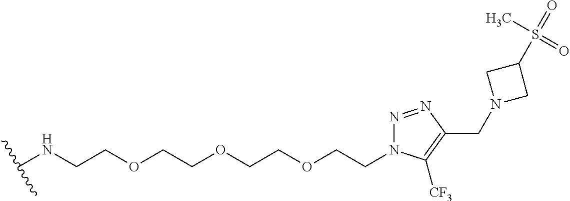 [SMILES: *NCCOCCOCCOCCn1nnc(CN2CC(S(C)(=O)=O)C2)c1C(F)(F)F] |
| 154 | 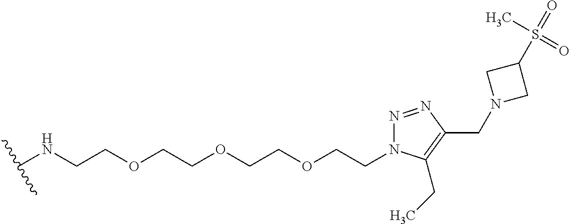 [SMILES: *NCCOCCOCCOCCn1nnc(CN2CC(S(C)(=O)=O)C2)c1CC] |

Conjugation of any of the compounds in Table 4 to a polymer (e.g., an alginate) may be performed as described in Example 2 of WO 2019/195055 or any other suitable chemical reaction.

In some embodiments, the compound is a compound of Formula (I) (e.g., Formulas (I-a), (1-b), (I-c), (I-d), (I-e), (I-f), (II), (II-a), (II), (III-a), (II-b), (III-c), or (III-d)), or a pharmaceutically acceptable salt thereof and is selected from:

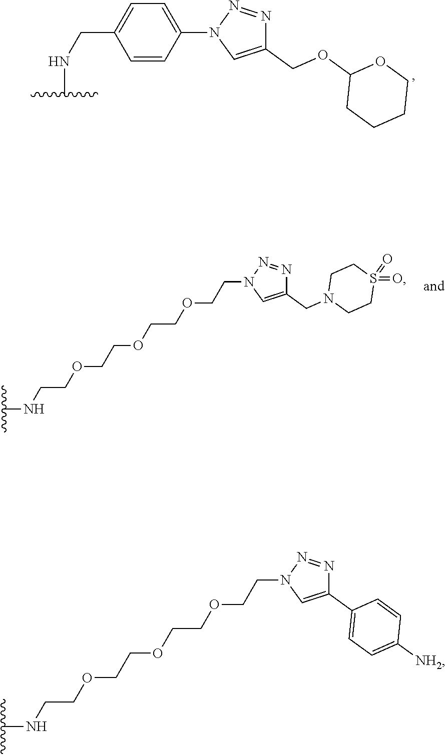 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In some embodiments, the device described herein comprises the compound of

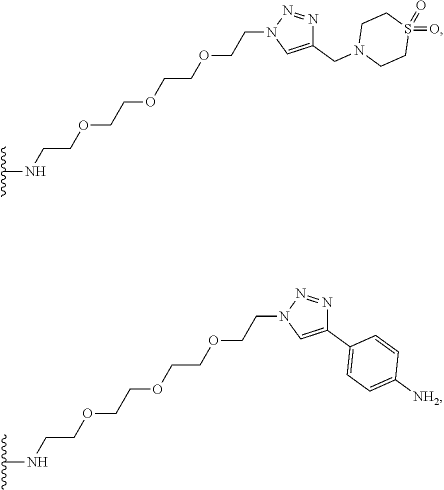 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.

In some embodiments, a compound of Formula (I) (e.g., Compound 101 in Table 4) is covalently attached to an alginate (e.g., an alginate with approximate MW<75 kDa, G:M ratio ≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429.

### Immunosuppressants

In some embodiments, the device is configured to locally release an immunosuppressant (e.g., as defined herein) during a desired release period after the device is implanted into a subject. In an embodiment, the desired release period is at least 30 days. In an embodiment, the immunosuppressant is continuously released throughout the release period by an extended release formulation present in the device.

The extended release formulation can comprise a solid, semi-solid, gel, or liquid form of the immunosuppressant. In addition, the extended release formulation may comprise the immunosuppressant in conjunction with another component, such as a polymer, solvent, or other excipient. The extended release formulations described herein may provide control over immunosuppressant release from the device; for example, the extended release formulation may liberate a small amount of immunosuppressant into or out of the device over time, e.g., to provide a substantially constant concentration of the immunosuppressant in or around the device and or subject over an extended period of time. In an embodiment, the extended release formulation provides a dosage of the immunosuppressant for longer than 1 day (e.g., greater than 2 days, 3 days, 4 days, 5 days, 6 days, 1 week, 1.5 weeks, 2 weeks, 2.5 weeks, 3 weeks, 3.5 weeks, 4 weeks, 5 weeks, 6 weeks, 2 months, 3 months or more), e.g., upon administration to a subject.

In an embodiment, the extended release formulation of the immunosuppressant is a solid (e.g., a crystal or a granule). In an embodiment, the extended release formulation of the immunosuppressant is a semi-solid or a gel. In an embodiment, the extended release formulation of the immunosuppressant is a liquid (e.g., a solution, e.g., an aqueous solution).

The extended release formulation of the immunosuppressant may comprise another component, such as a polymer. Exemplary polymers may be biodegradable or non-biodegradable. In an embodiment, the polymer is a synthetic biodegradable polymer, e.g., a polymer that is not naturally occurring. In an embodiment, the polymer is a naturally occurring biodegradable polymer, e.g., a polymer that is found in nature, e.g., a polypeptide or polysaccharide. Exemplary polymers include polyesters, polyethers, polycarbonates, polyvinyl alcohols, polyurethanes, polypropylenes, polyphosphazenes, polyanhydrides, alginate, dextran, hyaluronic acid, cellulose, xanthan gum, scleroglucan, and the like.

The polymer may be linear or branched. A branched polymer may be a star polymer, comb polymer, brush polymer, dendronized polymer, ladder, or dendrimer. In an embodiment, the polymer may be cross-linked. Further, the polymer may comprise selected molecular weight ranges, degrees of polymerization, viscosities or melt flow rates. The polymer may be thermoresponsive (e.g., a gel, e.g., which becomes a solid or liquid upon exposure to heat or a certain temperature) or can also be photocrosslinkable (e.g., a gel, e.g., which becomes a solid upon photocrosslinking).

In an embodiment, the polymer in an extended release formulation of the immunosuppressant has a molecular weight (Mw) greater than about 1,000 Da (e.g., greater than about 2,500 Da, 5,000 Da, 7,500 Da, 10,000 Da, 12,500 Da, 15,000 Da, 20,000 Da, 25,000 Da, 50,000 Da, or more). In an embodiment, the polymer has a Mw of less than about 100,000 Da (e.g., less than about 90,000 Da, 80,000 Da, 75,000 Da, 50,000 Da, 25,000 Da, 20,000 Da, 15,000 Da, 10,000 Da, or less). In an embodiment, the polymer has a Mw of between 1,000 Da and 100,000 Da. For example, the polymer may have a Mw between 1,000 Da and 50,000 Da, 2,500 Da and 40,000 Da, 5,000 Da and 30,000 Da, 5,000 Da and 20,000 Da, or 10,000 Da and 20,000. In an embodiment, the polymer has a Mw between 1,000 Da and 50,000 Da. In an embodiment, the polymer has a Mw between 5,000 Da and 20,000 Da.

The polymer may comprise any type of end group on its termini. For example, the polymer may comprise an amine, ester, acid, hydroxyl, acyl, amide, or ether end group on one or more of its termini. In an embodiment, the polymer comprises the same end group on each termini. In an embodiment, the polymer comprises a plurality of end groups on its termini (e.g., at least 2, 3, 4, 5 different end groups on its termini). In an embodiment, the polymer comprises an acid group on at least one of its termini. In an embodiment, the polymer comprises an ester group on at least one of its termini.

In an embodiment, the polymer in an extended release formulation of the immunosuppressant is a polyester. Polyesters degrade through the nonspecific hydrolysis of their ester bonds. Exemplary polyesters include poly(lactic acid) (PLA), poly(L-lactic acid) (PLLA), poly(D-lactic acid) (PDLA), poly(glycolic acid) (PGA), poly(lactic glycolic acid) (PLGA), poly(caprolactone) (PCL), and poly(lactic caprolactone) (PLCL). In an embodiment, the polyester polymer comprises lactic acid (e.g., a lactide). In an embodiment, the polyester polymer comprises a glycolic acid (e.g., a glycolide). In an embodiment, the polyester polymer comprises poly(lactic glycolic acid) (PLGA) (e.g., poly(D,L-lactic glycolic acid), poly(D-lactic glycolic acid), or poly(L-lactic glycolic acid)). The PLGA may be obtained from any commercial supplier or may be prepared de novo. In an embodiment, the PLGA is a Resomer R PLGA.

The polymer may be a homogeneous polymer or a blended polymer (e.g., a co-polymer of a block co-polymer). In the case of blended polymers, the polymer may comprise a plurality of monomer units (e.g., at least 2, 3, 4, 5, or 6 types of monomer units). In an embodiment, for blended polymers comprising 2 monomer units (Unit A and Unit B), the ratio of Unit A to Unit B is between 0.1:99.1 to 99.1:0.1. For example, the ratio of Unit A to Unit B may be 0.1:99.1, 0.5:99.5, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, 99:1, 99.5:0.5, or 99.1:0.1. Similarly, a blended polymer may comprise 3 monomer units (Unit A, Unit B, and Unit C) with exemplary ratios of Unit A to Unit B to Unit C between 1:1:99 to 99:1:1 (including varying amounts of Unit B).

In an embodiment, the polymer in an extended release formulation of the immunosuppressant has a viscosity (e.g., inherent viscosity) of between 0.01 dL/g and 2.5 dL/g. For example, the polymer may have a viscosity of between 0.05 dL/g and 2.0 dL/g, 0.1 dL/g to 0.5 dL/g, 0.1 dL/g to 0.3 dL/g, or 1.0 dL/g and 1.5 dL/g. In an embodiment, the polymer is free of a contaminant, such as a metal, catalyst, free radical, oxygen, microbe, or endotoxin. In an embodiment, the polymer is sterile. In an embodiment, the polymer is provided as a powder, granules, pellets, crystals, a gel, or a liquid.

The extended release formulation of the immunosuppressant may comprise another component, such as a solvent. The solvent may be an organic solvent that is miscible with water, or an organic solvent that is not miscible with water. In an embodiment, the solvent is an organic solvent that aids in the solubility and stability of the immunosuppressant over time. In an embodiment, the organic solvent is any organic solvent approved by the Food and Drug Administration (FDA) for use in humans. Exemplary organic solvents include dimethylamide (DMA), dimethylformamide (DMF), N-methyl-2-pyrrolidone (NMP), dimethylsulfoxide (DMSO), formamide, tetrahydrofuran (THF), ethanol, isopropanol, acetone, ethyl acetate, 1,4-dioxane, pyridine, and triethylamine (TEA). In an embodiment, the organic solvent comprises an amine (e.g., a primary amine, secondary amine, or tertiary amine). In an embodiment, the organic solvent comprises a ketone. In an embodiment, the organic solvent has a boiling point greater than about 100° C., e.g., greater than about 150° C., 200° C., 250° C., 300° C. or higher.

Exemplary extended release formulations described herein may comprise a both a polymer and an organic solvent, e.g., at a fixed ratio of polymer to organic solvent. Factors such as solubility, stability, toxicity, or volume of the final composition are often taken into consideration when determining the ratio of the polymer with the organic solvent. In one embodiment, the ratio of the polymer to the organic solvent is between 50:50 and 90:10 (w/w) (e.g., 50:50, 60:40, 70:30, 80:20, or 90:10). In another embodiment, the ratio of the polymer to the organic solvent is between 10:90 and 50:50 (w/w) (e.g., 10:90, 20:80, 30:70, 40:60, or 50:50).

The concentration of immunosuppressant in the extended release formulation may be within the range of 0.01% to 50% w/w (e.g., about 0.1% to 40% w/w or 1% to 25% w/w). In some embodiments, the concentration of immunosuppressant in the extended release formulation is about 0.1% to 25% w/w, e.g., between about 0.1% and 20%, about 0.1% and 15%, about 0.1% and 10%, about 0.5% and 25%, about 0.5% and 20%, about 0.5% and 15%, about 0.5% and 10%, about 1% and 25%, about 1% and 20%, about 1% and 15%, or about 1% and 10% w/w). In some embodiments, the concentration of immunosuppressant in the extended release formulation is about 0.05%, about 0.1%, about 0.5%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11%, about 11.5%, about 12%, about 12.5%, about 13%, about 13.5%, about 14%, about 14.5%, about 15%, about 17.5%, about 20%, or about 25% w/w.

The concentration of immunosuppressant in the extended release formulation may be within the range of 0.01% to 50% w/v (e.g., about 0.1% to 40% w/v or 1% to 25% w/v). In some embodiments, the concentration of immunosuppressant in the extended release formulation is about 0.1% to 25% w/v, e.g., between about 0.1% and 20%, about 0.1% and 15%, about 0.1% and 10%, about 0.5% and 25%, about 0.5% and 20%, about 0.5% and 15%, about 0.5% and 10%, about 1% and 25%, about 1% and 20%, about 1% and 15%, or about 1% and 10% w/v). In some embodiments, the concentration of immunosuppressant in the extended release formulation is about 0.05%, about 0.1%, about 0.5%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10%, about 10.5%, about 11%, about 11.5%, about 12%, about 12.5%, about 13%, about 13.5%, about 14%, about 14.5%, about 15%, about 17.5%, about 20%, or about 25% w/v.

The extended release formulations compositions provided are formulated for sustained or delayed release of an immunosuppressant described herein (e.g., a compound of Formula (I)). Sustained release, as described herein, refers to the property of a composition by which release of an immunosuppressant from the extended release formulation occurs over an extended period of time as compared to the release from an isotonic saline solution. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the immunosuppressant or any other material associated with the sustained release formulation. To illustrate further, a wide range of degradation rates or release rates may be obtained by adjusting the features of the extended release formulation, such as the identity of the polymer or any related feature of the polymer (including the molecular weight, viscosity, end group, etc.), the identity of the organic solvent, the concentration of any one of the polymer, organic solvent, or immunosuppressant, or ratios thereof.

One protocol generally accepted in the field that may be used to determine the release rate of the immunosuppressant from an extended release formulation described herein entails disposing the extended release formulation into a physiological environment and removing samples of the mixture and various time points for analysis, e.g., by HPLC.

The release rate of the immunosuppressant from the extended release formulation may also be characterized by the amount of immunosuppressant released per day per quantity of polymer present in the sustained release formulation. For example, in some embodiments, the release rate may vary from about 1 ng or less of the immunosuppressant per day per quantity of polymer to about 500 or more ng/day/quantity of polymer. Alternatively, the release rate may be about 0.05, 0.5, 5, 10, 25, 50, 75, 100, 125, 150, 175, 200, 250, 300, 350, 400, 450, or 500 ng/day/quantity of polymer. In still other embodiments, the release rate of the immunosuppressant may be 10,000 ng/day/ng/day/quantity of polymer, or even higher.

In an embodiment, the release of immunosuppressant from an extended release formulation described herein may be described as the half-life (i.e., T_{1/2}) of such material in the formulation.

In an embodiment, an extended release formulation described herein provides a dosage of the immunosuppressant for greater than 1 day (e.g., greater than 2 days, 3 days, 4 days, 5 days, 6 days, 1 week, 1.5 weeks, 2 weeks, 2.5 weeks, 3 weeks, 3.5 weeks, 4 weeks, 5 weeks, 6 weeks, 2 months, 3 months or more), e.g., upon administration of the device to a subject. In an embodiment, the extended release formulation provides a dosage of the immunosuppressant for greater than 1 week (e.g., greater than 1.5 weeks, 2 weeks, 2.5 weeks, 3 weeks, 3.5 weeks, 4 weeks, 5 weeks, 6 weeks, 2 months, 3 months or more), e.g., upon administration to a subject. In an embodiment, the extended release formulation provides a dosage of the immunosuppressant between 1 week and 10 weeks (e.g., 1 week and 8 weeks, 1 week and 6 weeks, 1 week and 4 weeks, 2 weeks and 8 weeks, 3 weeks and 6 weeks), e.g., upon administration to a subject. In an embodiment, the extended release formulation provides a dosage of the immunosuppressant between 0.5 ug and 500 ug per day (e.g., between 1 ug and 500 ug, 1 ug and 250 ug, 1 ug and 100 ug, 1 ug and 50 ug, 5 ug and 500 ug, 5 ug and 250 ug, 5 ug and 100 ug, 5 ug and 50 ug, 10 ug and 250 ug, 10 ug and 100 ug, 10 ug and 50 ug, 50 ug and 500 ug, 50 ug and 250 ug, 50 ug and 100 ug).

In an embodiment, the extended release formulation has the property that it forms a drug depot within a device described herein. A drug depot refers to a localized mass of a particular immunosuppressant (e.g., solid particles of a glucocorticoid described herein), wherein the immunosuppressant is gradually released from the device, e.g., by diffusion. A drug depot may comprise at least one region which provides a bolus of the immunosuppressant, and a remaining region which provides a sustained release of the immunosuppressant.

In an embodiment, the extended release formulation comprises a plurality of particles of the immunosuppressant suspended in a hydrogel in the device, e.g., within a hydrogel that forms the outer compartment or the inner, cell-containing compartment of a two-compartment device described herein. In an embodiment, the particles comprise the immunosuppressant in crystalline form.

In an embodiment, the immunosuppressant particles are prepared by adding a desired quantity of an amorphous powder of the immunosuppressant (e.g., a glucocorticoid) to a desired volume of a solution comprising a hydrogel-forming polymer (e.g., an alginate or alginate mixture described herein), sonicating the resulting powder-polymer mixture until a substantially homogenous suspension is formed, and contacting droplets of the resulting suspension with a cross-linking solution. In an embodiment, the quantity of the glucocorticoid powder and the volume of the polymer solution are selected to achieve a mixture of 2.5 mg to 5.0 mg powder per mL polymer solution. In an embodiment, the genetically-modified cells are added to the homogenous suspension and the resulting cell-containing suspension is used to form the inner compartment of a two-compartment hydrogel capsule as described herein. In an embodiment, the glucocorticoid is triamcinolone hexacetonide, fluticasone furoate, fluticasone propionate or mometasone furoate.

### Glucocorticoid Compounds

In some embodiments, the immunosuppressant in an extended release formulation is glucocorticoid. In some embodiments, the glucocorticoid is a compound of Formula (IV):

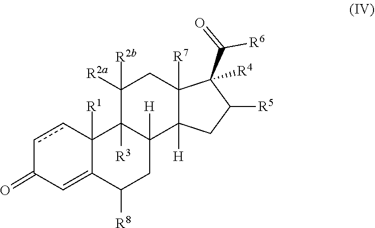

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃). In some embodiments, R⁸ is hydrogen. In some embodiments, [symbol] is a single bond. In some embodiments, [symbol] is a double bond.

In some embodiments, the glucocorticoid compound of Formula (IV) is a compound of Formula (IV-a):

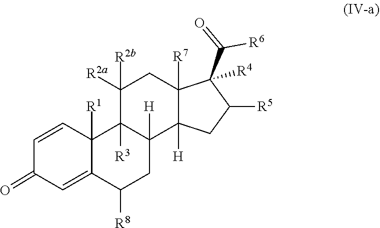

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}, and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃). In some embodiments, R⁸ is hydrogen.

In some embodiments, the glucocorticoid compound of Formula (IV) is a compound of Formula (IV-b):

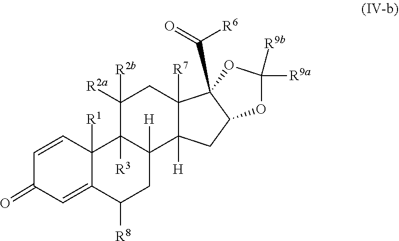

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; each of R⁹ª and R^{9b} is independently halo, C₁-C₆ alkyl, or —OR^{A}, and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, each of R⁹ª and R^{9b} is independently C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl, In some embodiments, R⁶ is C₁-C₆ heteroalkyl (e.g., CH₂OH, CH₂CH₂OH). In some embodiments, R⁷ is C₁-C₆ alkyl (e.g., CH₃). In some embodiments, R⁸ is hydrogen.

In some embodiments, the glucocorticoid compound of Formula (IV) is a compound of Formula (IV-c):

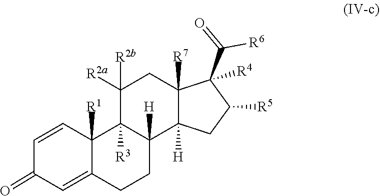

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁹ is halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃).

In some embodiments, the glucocorticoid compound of Formula (IV) is a compound of Formula (IV-d):

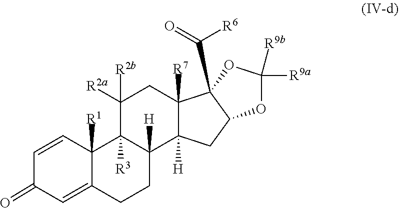

or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; each of R⁹ª and R^{9b} is independently halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁴ and R⁵ is independently —OR^{A}. In some embodiments, each of R⁹ª and R^{9b} is independently C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH). In some embodiments, R⁷ is C₁-C₆ alkyl (e.g., CH₃).

In some embodiments, the glucocorticoid compound of Formula (IV) is a compound of Formula (IV-e):

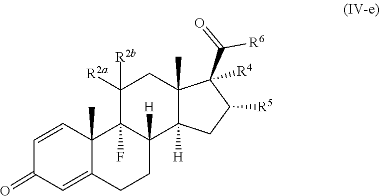

or a pharmaceutically acceptable salt, ester, hydrate, tautomer, or prodrug thereof, wherein each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁹ is halo, C₁-C₆ alkyl, or —OR^{A}; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

In some embodiments, one of R^{2a} and R^{2b} is independently —OR^{A} and the other of R^{2a} and R^{2b} is independently hydrogen. In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁴ and R⁵ are taken together to form a 5-membered ring substituted by 2 R⁹. In some embodiments, R⁹ is C₁-C₆ alkyl (e.g., CH₃). In some embodiments, R⁶ is C₁-C₆ alkyl, C₁-C₆ alkenyl, C₁-C₆ alkynyl, or C₁-C₆ heteroalkyl. In some embodiments, R⁶ is C₁-C₆ heteroalkyl (e.g., CH₂OH, CH₂CH₂OH).

In some embodiments, the glucocorticoid compound of Formula (IV) is a compound of Formula (IV-f):

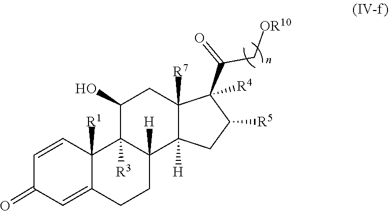

or a pharmaceutically acceptable salt, ester, hydrate, tautomer, or prodrug thereof, wherein R¹ is hydrogen, halo, or C₁-C₆ alkyl; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁹ is halo, C₁-C₆ alkyl, or —OR^{A}; R¹⁰ is hydrogen or C₁-C₆ alkyl; each of R^{A}, R^{B}, R^{C}, R^{D}, and R^{E} is independently hydrogen or C₁-C₆ alkyl; and n is 0, 1, 2, 3, or 4.

In some embodiments, R¹ is C₁-C₆ alkyl (e.g., CH₃). In some embodiments, R³ is halogen (e.g., fluoro). In some embodiments, each of R⁴ and R⁵ is independently —OR^{A}. In some embodiments, R⁴ and R⁵ are taken together to form a ring (e.g., a 5-membered ring) substituted by one or more R⁹. In some embodiments, R⁷ is C₁-C₆ alkyl (e.g., CH₃). In some embodiments, R+ and R⁵ are taken together to form a 5-membered ring substituted by 2 R⁹. In some embodiments, R⁹ is C₁-C₆ alkyl (e.g., CH₃). In some embodiments, R¹⁰ is hydrogen. In some embodiments, n is 1.

In some embodiments, the glucocorticoid compound of Formula (IV) is a compound of Formula (IV-g):

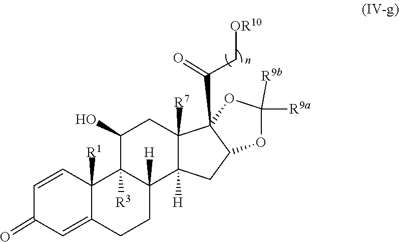

or a pharmaceutically acceptable salt, ester, hydrate, tautomer, or prodrug thereof, wherein R¹ is hydrogen, halo, or C₁-C₆ alkyl; R³ is hydrogen, halo, or C₁-C₆ alkyl; R⁷ is hydrogen, halo, or C₁-C₆ alkyl; each of R⁹ª and R^{9b} is independently halo, C₁-C₆ alkyl, or —OR^{A}; R¹⁰ is hydrogen or C₁-C₆ alkyl; each of R^{A}, R^{B}, R^{C}, R^{D}, and R^{E} is independently hydrogen or C₁-C₆ alkyl; and n is 0, 1, 2, 3, or 4.

In some embodiments, R¹ is C₁-C₆ alkyl (e.g., CH₃). In some embodiments, one of R^{2a} and R^{2b} is independently —OR^{A} and the other of R^{2a} and R^{2b} is independently hydrogen. In some embodiments, R³ is halogen (e.g., fluoro). In some embodiments, each of R⁹ª and Rob is independently C₁-C₆ alkyl (e.g., CH₃). In some embodiments, R⁷ is C₁-C₆ alkyl (e.g., CH₃). In some embodiments, R¹⁰ is hydrogen. In some embodiments, n is 1.

In some embodiments, the glucocorticoid compound of Formula (IV) is triamcinolone or a pharmaceutically acceptable salt, ester, hydrate, tautomer, or prodrug thereof. In some embodiments, the glucocorticoid compound of Formula (IV) is triamcinolone acetate or a pharmaceutically acceptable salt, ester, hydrate, tautomer, or prodrug thereof. In some embodiments, the glucocorticoid compound of Formula (IV) is

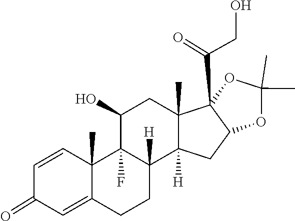

or a pharmaceutically acceptable salt, ester, hydrate, tautomer, or prodrug thereof. In some embodiments, the glucocorticoid compound of Formula (IV) is triamcinolone hexacetonide or a pharmaceutically acceptable salt, ester, hydrate, tautomer, or prodrug thereof. In some embodiments, the glucocorticoid compound of Formula (IV) is

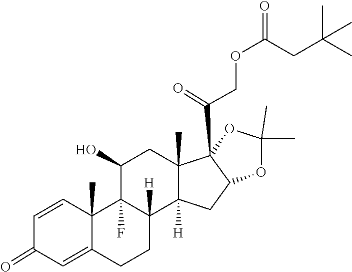

or a pharmaceutically acceptable salt, ester, hydrate, tautomer, or prodrug thereof. In some embodiments, the glucocorticoid is any of the compounds described in U.S. Pat. No. 2,990,401, which is incorporated herein by reference.

In some embodiments, the glucocorticoid compound of Formula (IV) is fluticasone or a pharmaceutically acceptable salt, ester, hydrate, tautomer, or prodrug thereof. In some embodiments, the glucocorticoid compound of Formula (IV) is fluticasone furoate or a pharmaceutically acceptable salt, ester, hydrate, tautomer, or prodrug thereof. In some embodiments, the glucocorticoid compound of Formula (IV) is

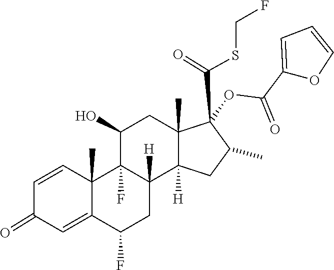

or a pharmaceutically acceptable salt, ester, hydrate, tautomer, or prodrug thereof. In some embodiments, the glucocorticoid is any of the compounds described in U.S. Pat. No. 7,101,866, which is incorporated herein by reference. In some embodiments, the glucocorticoid compound of Formula (IV) is fluticasone propionate or a pharmaceutically acceptable salt, ester, hydrate, tautomer, or prodrug thereof. In some embodiments, the glucocorticoid compound of Formula (IV) is

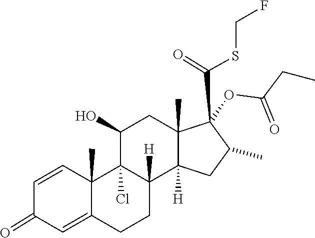

or a pharmaceutically acceptable salt, ester, hydrate, tautomer, or prodrug thereof. In some embodiments, the glucocorticoid is any of the compounds described in U.S. Pat. No. 4,335,121, which is incorporated herein by reference.

In some embodiments, the glucocorticoid compound of Formula (IV) is mometasone or a pharmaceutically acceptable salt, ester, hydrate, tautomer, or prodrug thereof. In some embodiments, the glucocorticoid compound of Formula (IV) is mometasone furoate or a pharmaceutically acceptable salt, ester, hydrate, tautomer, or prodrug thereof. In some embodiments, the glucocorticoid compound of Formula (IV) is

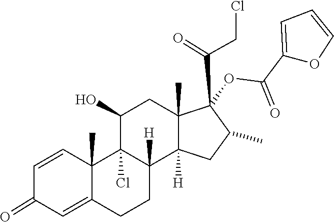

or a pharmaceutically acceptable salt, ester, hydrate, tautomer, or prodrug thereof. In some embodiments, the glucocorticoid is any of the compounds described in U.S. Pat. No. 4,472,393, which is incorporated herein by reference.

In some embodiments, the glucocorticoid compound of Formula (IV) is beclomethasone or a pharmaceutically acceptable salt, ester, hydrate, tautomer, or prodrug thereof. In some embodiments, the glucocorticoid compound of Formula (IV) is beclomethasone dipropionate or a pharmaceutically acceptable salt, ester, hydrate, tautomer, or prodrug thereof. In some embodiments, the glucocorticoid compound of Formula (IV) is

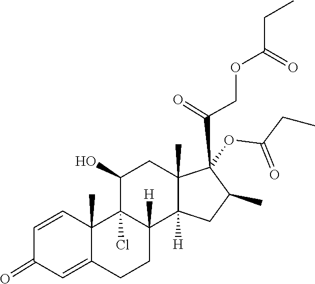

or a pharmaceutically acceptable salt, ester, hydrate, tautomer, or prodrug thereof. In some embodiments, the glucocorticoid is any of the compounds described in U.S. Pat. No. 3,645,590, which is incorporated herein by reference.

### Rapamycin Compounds and Particles

In some embodiments, the immunosuppressant in an extended release formulation is rapamycin, an analogue or derivative of rapamycin, or a pharmaceutically acceptable salt of rapamycin, analogue or derivative. The structure of rapamycin is:

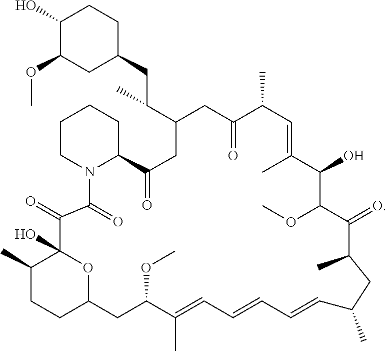 [SMILES: COC1C(=O)[C@H](C)C[C@H](C)/C=C/C=C/C=C(\C)[C@@H](OC)CC2CC[C@@H](C)[C@@](O)(O2)C(=O)C(=O)N2CCCC[C@H]2C(=O)CC([C@H](C)C[C@@H]2CC[C@@H](O)[C@H](OC)C2)CC(=O)[C@H](C)/C=C(\C)[C@H]1O]

The term rapamycin compound as used herein refers to rapamycin and compounds with a structural similarity to rapamycin, e.g., compounds with a similar macrocyclic structure which have been modified to enhance therapeutic benefit. In an embodiment, the mTOR inhibitor is rapamycin or the rapamycin derivative everolimus. In an embodiment, the mTOR inhibitor is the rapamycin ester cell cycle inhibitor-779 (CCI-779). Additional rapamycin compounds which may be used in the devices described herein include but are not limited to the rapamycin analogues and derivatives described in any of the following U.S. Pat. Nos. 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100, 883; 5,023,264; 5,023,263; and 5,023,262, each of which is incorporated herein by reference in its entirety.

Any of the rapamycin compounds described herein may be used to prepare rapamycin particles for encapsulation in hydrogel capsules. In an embodiment, rapamycin particles are prepared using rapamycin.

In an embodiment, the rapamycin particles are biodegradable nanoparticles described in WO2021183781 or WO2016073799.

### Preparation of Two-Compartment Hydrogel Capsules

Each compartment of a device described herein may comprise an unmodified polymer, a polymer modified with a compound of Formula (I), or a blend thereof. Briefly, to prepare a device configured as a two-compartment hydrogel capsule, a volume of a first polymer solution (e.g., comprising an unmodified polymer, a polymer modified with a compound of Formula (I), or a blend thereof, and optionally containing cells,) is loaded into a first syringe connected to the inner lumen of a coaxial needle. The first syringe may then be connected to a syringe pump oriented vertically above a vessel containing an aqueous cross-linking solution which comprises a cross-linking agent, a buffer, and an osmolarity-adjusting agent. A volume of the second polymer solution (e.g., comprising an unmodified polymer, a polymer modified with a compound of Formula (I), or a blend thereof, and optionally containing cells) is loaded into a second syringe connected to the outer lumen of the coaxial needle. The second syringe may then be connected to a syringe pump oriented horizontally with respect to the vessel containing the cross-linking solution. A high voltage power generator may then be connected to the top and bottom of the needle. The syringe pumps and power generator can then be used to extrude the first and second polymer solutions through the syringes with settings determined to achieve a desired droplet rate of polymer solution into the cross-linking solution. The skilled artisan may readily determine various combinations of needle lumen sizes, voltage range, flow rates, droplet rate and drop distance to create 2-compartment hydrogel capsule compositions in which the majority (e.g., at least 80%, 85%, 90% or more) of the capsules are within 10% of the target size and desired shape. After exhausting the first and second volumes of polymer solution, the droplets may be allowed to cross-link in the cross-linking solution for certain amount of time, e.g., about five minutes.

An exemplary process for preparing a composition of millicapsules (e.g., 1.5 mm diameter) is described in WO 2019/195055.

### Device Compositions

A device described herein may be provided as a preparation or composition for implantation or administration to a subject, e.g., a subject with an IMID. In some embodiments, a device preparation or composition comprises at least 2, 4, 8, 16, 32, 64 or more devices, and at least 20%, 30%, 35%, 40%, 45%, 50%, 55%, 60%, 65%, 70%, 75%, 80%, 85%, 90%, 95% or 100% of the devices in the preparation or composition have a characteristic as described herein, e.g., mean diameter or mean pore size or cell density.

A device composition may be configured for implantation, or implanted or disposed into or onto any site of the body. In some embodiments, a device, preparation or composition is configured for implantation, implanted, or disposed into the subcutaneous fat of a subject, or into the muscle tissue of a subject. In some embodiments, the device, device preparation or device composition is configured for implantation or implanted or disposed into the peritoneal cavity (e.g., the omentum). In some embodiments, the device is configured for implantation or implanted or disposed into or onto the lesser sac, also known as the omental bursa or bursalis omentum. The lesser sac refers to a cavity located in the abdomen formed by the omentum, and is in close proximity to, for example, the greater omentum, lesser omentum, stomach, small intestine, large intestine, liver, spleen, gastrosplenic ligament, adrenal glands, and pancreas. Typically, the lesser sac is connected to the greater sac via the omental foramen (i.e., the Foramen of Winslow). In some embodiments, the lesser sac comprises a high concentration of adipose tissue. A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, preparation or composition into the omentum (e.g., the lesser sac) are provided in M. Pellicciaro et al. (2017) CellR4 5(3):e2410.

In some embodiments, a device or preparation is easily retrievable from a subject, e.g., without causing injury to the subject or without causing significant disruption of the surrounding tissue. In an embodiment, the device or preparation can be retrieved with minimal or no surgical separation of the device(s) from surrounding tissue, e.g., via minimally invasive surgical approach, extraction, or resection.

A device, composition or preparation can be configured to provide continuous delivery of an immunomodulatory protein for a variety of time periods after implant into a mammalian recipient (e.g., a human patient), including: a short continuous delivery (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less) or prolonged delivery (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or longer).

In some embodiments, the device composition or preparation does not contain any capsule, device, implant or other object disclosed in any of WO2012/112982, WO2012/167223, WO2014/153126, WO2016/019391, WO2016/187225, WO 2018/232027, WO 2019/068059, WO 2019/169089, US2012-0213708, US 2016-0030359, and US 2016-0030360.

### IMID Therapies

The devices and compositions/preparations thereof may be useful in treating a variety of IMIDs. Selection of an immunomodulatory protein(s) and optional immunosuppressant to be produced by a device for treating a particular IMID would typically include consideration of the etiology of the IMID and the biological activities of the immunomodulatory protein and immunosuppressant.

Table 5A below lists exemplary immunomodulatory proteins and optional immunosuppressants that may be useful for treating particular IMIDs when continuously delivered by administration of a device or device composition described herein.

TABLE 5A
Exemplary IMID Therapies

| Therapeutic Molecules | |
|---|---|
| Delivered by Device | IMID |
| IL-10 | AIH, Psoriasis |
| IL-10 + TAH | AIH, RA |
| IL-22 | AIH, IBD |
| IL-22 + cyclosporine | AIH, IBD |
| IL-22 + rapamycin | AIH, IBD |
| IL-22 + TAH | AIH, IBD |
| IL-22 + IL-10 | AIH, IBD |
| IL-22 + IL-10 + TAH | AIH, IBD |
| IL-22 + IL-10 + cyclosporine | AIH, IBD |
| IL-22 + IL-10 + rapamycin | AIH, IBD |
| sCTLA-4-Ig | GvHD, Sjogren's Syndrome, |
| | rheumatoid arthritis (RA) |
| sCTLA-4-Ig + TAH | GvHD, RA |
| sCTLA-4-Ig + cyclosporine | GvHD |
| sCTLA-4-Ig + rapamycin | GvHD |

Table 5B below lists exemplary IMID conditions and diseases that could be treated by administration (implantation) of a device or device composition that produces the indicated immunomodulatory protein. In an embodiment the device also releases an immuosuppressant (e.g., TAH, rapamycin or cyclosporine) from an extended release formulation contained in the device or in the device composition. In another embodiment, a patient with a specific IMID is treated by co-administration of a device producing one or more of the indicated immunomodulatory protein(s) and an immunosuppressant (e.g., in an extended release formulation).

TABLE 5B
Exemplary Immunomodulatory Therapies and IMID Conditions/Diseases

| Immunomodulatory | Liver | Rheumatoid | Multiple | Inflammatory | | |
|---|---|---|---|---|---|---|
| Protein | Disease | Arthritis | Sclerosis | Bowel Disease | Skin | GvHD |
| CTLA-4-Ig | + | + | | | | + |
| IL-1Ra | | + | | | | |
| IL-2 mutein | | | | | + | |
| IL-10 | + | + | + | + | | |
| IL-10-Fc | + | + | + | + | | |
| IL-22 | + | | + | + | | |
| IL-22-Fc | + | | | | | |
| PD-L1-Fc | | | | + | | |

### ENUMERATED EXEMPLARY EMBODIMENTS

1. An implantable device comprising a first plurality of mammalian cells (e.g., human cells) are genetically modified to express and secrete one or more immunomodulatory proteins, wherein the device is configured to exhibit the following properties when implanted into a subject:
(a) the subject's immune cells do not contact the genetically modified cells;
(b) the genetically modified cells do not exit the device; and
(c) continuously deliver each immunomodulatory protein to the subject in an amount and for a time period effective to induce an anti-inflammatory immune response in the subject.

2. The device of embodiment 1, which comprises at least one of the following features:
(i) the device comprises a second plurality of mammalian cells genetically modified to express and secrete at least one immunomodulatory protein that is different than each immunomodulatory protein secreted by the first plurality of cells;
(ii) an extended release formulation of an immunosuppressant;
(iii) at least one of the immunomodulatory proteins secreted by the first plurality of genetically modified cells comprises a heterologous secretory signal peptide sequence;
(iv) a compound or polymer disposed on the exterior surface of the device that mitigates the foreign body response (FBR) to the device;
(v) the surface of the device does not contain alginate; and
(vi) the cells in the first plurality of genetically modified mammalian cells are derived from ARPE-19 cells or from an induced pluripotent stem cell line.

3. The implantable device of embodiment 1 or 2, wherein the time period is at least any of 30 days, 60 days, 90 days 120 days, 180 days or longer.

4. The implantable device of any one of embodiments 1 to 3, wherein the anti-inflammatory immune response comprises one or both of: (x) increased expression of an anti-inflammatory cytokine in the subject's plasma; and (y) reduced expression of a pro-inflammatory cytokine in the subject's plasma.

5. The implantable device of any one of embodiments 1 to 4, wherein the anti-inflammatory immune response comprises increased expression of IL-10.

6. The implantable device of any one of embodiments 1 to 5, wherein the anti-inflammatory immune response comprises reduced expression of TNF-alpha.

7. The implantable device of any one of embodiments 1 to 6, wherein the anti-inflammatory immune response comprises reduced expression of interferon (IFN)-γ).

8. The implantable device of any one of embodiments 2 to 7, which comprises feature (i).

9. The implantable device of any one of embodiments 2 to 8, which comprises feature (ii).

10. The implantable device of any one of embodiments 2 to 10, which comprises feature (iii).

11. The implantable device of embodiment 10, wherein the heterologous signal peptide sequence consists essentially of MGWRAAGALLLALLLHGRLLA (SEQ ID NO:21).

12. The implantable device of any one of embodiments 2 to 11, which comprises feature (iv). 13. The implantable device of any one of embodiments 2 to 12, which comprises feature (v).

14. The implantable device of any one of embodiments 2 to 13, which comprises feature (vi).

15. The implantable device of any one of embodiments 1 to 14, wherein the immunomodulatory protein is an IL-10 protein, optionally an IL-10-Fc fusion protein.

16. The implantable device of any one of embodiments 1 to 14, wherein the immunomodulatory protein is an IL-22 protein, optionally an IL-22-Fc fusion protein.

17. The implantable device of embodiment 1 to 14, wherein the immunomodulatory protein is an IL-2 mutein protein, optionally an IL-2 mutein fusion protein.

18. The implantable device of any one of embodiments 1 to 14, wherein the immunomodulatory protein is a sCTLA-4 protein, optionally a sCTLA-4-Ig.

19. The implantable device of embodiment 18, wherein the mammalian cells encode the amino acid sequence shown in FIG. 3B.

20. The implantable device of embodiment 1 to 14, wherein the immunomodulatory protein is a sPD-L1 protein, optionally a PD-L1-Ig fusion protein.

21. The implantable device of embodiment 20, wherein the mammalian cells encode the amino acid sequence shown in FIG. 7A.

22. The implantable device of embodiment 14, wherein the immunomodulatory protein is an IL-1Ra protein, optionally an IL-1Ra-Ig fusion protein.

23. The implantable device of any one of embodiments 1 to 22, wherein each plurality of genetically modified cells is contained in a cell-containing compartment surrounded by a barrier compartment.

24. The implantable device of embodiment 23, wherein the cell-containing compartment comprises a first hydrogel-forming polymer and the barrier compartment comprises a second hydrogel-forming polymer.

25. The implantable device of embodiment 24, wherein one or both of the first hydrogel-forming polymer and the second hydrogel forming polymer is an alginate.

26. The implantable device of any one of embodiments 1 to 25, which comprises two or more cell-containing compartments.

27. The implantable device of any one of the above embodiments, wherein the device is configured to deliver an immunosuppressant compound to the subject for at least 20 days or at least 30 days.

28. The implantable device of any one of the above embodiments, wherein all the genetically modified cells in the device are derived from ARPE-19 cells.

29. The implantable device of any one of embodiments 1 to 27, wherein all the genetically modified cells in the device are derived from induced pluripotent stem cells.

30. A device composition comprising a preparation of devices and a pharmaceutically acceptable excipient, wherein each device in the preparation is a device as defined in any of the above embodiments.

31. A hydrogel capsule comprising:
(a) a cell-containing compartment which comprises living cells encapsulated in a first polymer composition, wherein at least a portion of the living cells (e.g., mammalian cells, e.g., human cells) are genetically modified to continuously express and secrete a first immunomodulatory protein; and
(b) a barrier compartment surrounding the cell-containing compartment and comprising a second polymer composition which comprises an alginate covalently modified with an at least one compound that mitigates the FBR;
wherein the hydrogel capsule has a spherical shape and has a diameter of 0.5 millimeter to 5 millimeters and optionally wherein the barrier compartment has an average thickness of about 10 to about 300 microns, about 20 to about 150 microns, or about 40 to about 75 microns.

32. The hydrogel capsule of embodiment 31, wherein the cell-containing compartment comprises living cells genetically modified to express and secrete a second immunomodulatory protein. 33. The hydrogel capsule of embodiment 32, wherein the first and second immunomodulatory proteins are expressed by the same cells.

34. The hydrogel capsule of any one of embodiments 31 to 33, which further comprises an extended release formulation or an immunosuppressant compound in one or both of the cell-containing compartment and the barrier compartment, optionally wherein the immunosuppressant compound is a glucocorticoid compound or a rapamycin compound.

35. The hydrogel capsule of embodiment 34, wherein the first polymer composition comprises a hydrogel-forming polymer and the extended release formulation of the immunosuppressant compound is prepared by a process which comprises adding a desired quantity of an amorphous powder of the immunosuppressant compound to a desired volume of a solution comprising the hydrogel-forming polymer, sonicating the resulting mixture until a substantially homogenous suspension is formed, adding the living cells to the suspension and contacting droplets of the polymer, immunosuppressant compound and cell suspension with a cross-linking solution, optionally wherein the hydrogel-forming polymer is an alginate.

36. The hydrogel capsule of any one of embodiments 31 to 35, wherein the immunomodulatory protein is an IL-10 protein, optionally an IL-10-Fc fusion protein.

37. The hydrogel capsule of any one of embodiments 31 to 35, wherein the immunomodulatory protein is an IL-22 protein, optionally an IL-22-Fc fusion protein.

38. The hydrogel capsule of any one of embodiments 31 to 35, wherein the immunomodulatory protein is an IL-2 mutein protein, optionally an IL-2 mutein fusion protein.

39. The hydrogel capsule of any one of embodiments 31 to 35, wherein the immunomodulatory protein is a sCTLA-4 protein, optionally a sCTLA-4-Ig.

40. The hydrogel capsule of embodiment 39, wherein the mammalian cells encode the amino acid sequence shown in FIG. 3B.

41. The hydrogel capsule of any one of embodiments 31 to 35, wherein the immunomodulatory protein is a sPD-L1 protein, optionally a PD-L1-Ig fusion protein.

42. The hydrogel capsule of embodiment 41, wherein the mammalian cells encode the amino acid sequence shown in FIG. 7A.

43. The hydrogel capsule of any one of embodiments 31 to 35, wherein the immunomodulatory protein is an IL-1Ra protein, optionally an IL-1Ra-Ig fusion protein.

44. The hydrogel capsule of any one of embodiments 31 to 35, wherein all of the genetically modified cells in the capsule are derived from ARPE-19 cells.

45. The hydrogel capsule of any one of embodiments 31 to 35, wherein all of the genetically modified cells in the capsule are derived from induced pluripotent stem cells.

46. A device composition comprising a preparation of hydrogel capsules and a pharmaceutically acceptable excipient, wherein each hydrogel capsule in the preparation is a hydrogel capsule as defined in any of embodiments 31 to 45, and optionally wherein the composition has a volume of less than 10 milliliters, less than 8 ml, or less than 5 ml.

47. A method of providing an immunomodulatory protein to a subject diagnosed with an IMID, comprising administering to the subject the device of any one of embodiments 1 to 29, the hydrogel composition of any one of embodiments 31 to 46, or the device composition of embodiment 30 or 46.

48. The method of embodiment 47, further comprising assaying a plasma sample from the subject for an anti-inflammatory response.

49. The method of embodiment 47 or 48, wherein the plasma sample is collected at two or more time points following the administration, optionally wherein the time points are selected from the group consisting of day 7, day 14, day 21, day 28, day 35, day 42 and day 48.

50. The method of any one of embodiments 47 to 49, wherein the IMID is an inflammatory liver disease, optionally auto-immune hepatitis.

51. The method of any one of embodiments 47 to 49, wherein the IMID is an auto-immune disease, optionally rheumatoid arthritis.

52. The method of any one of embodiments 47 to 49, wherein the IMID is graft versus host disease. 53. The method of any one of embodiments 47 to 49, wherein the IMID is inflammatory bowel disease.

54. The method of any one of embodiments 47 to 49, wherein the IMID is multiple sclerosis.

55. The method of any one of embodiments 47 to 49, wherein the IMID is an inflammatory skin disease, optionally psoriasis.

56. The implantable device, hydrogel capsule, device composition or method of any one of the above embodiments, wherein the FBR-mitigating compound is a compound of Formula (I):

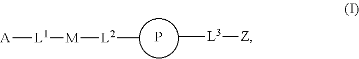

or a pharmaceutically acceptable salt thereof, wherein:
A is alkyl, alkenyl, alkynyl, heteroalkyl, cycloalkyl, heterocyclyl, aryl, heteroaryl, —O—, C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{E}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E}1, S(O)ₓR^{E1}, OS(O)ₓR^{E1}, —N(R^{C1})S(O)ₓR^{E1}, S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

57. The implantable device, hydrogel capsule, device composition or method of any one of the above embodiments, wherein the FBR-mitigating compound is selected from the compounds shown in Table 4 above, or a pharmaceutically acceptable salt of the selected compound.

58. The implantable device, hydrogel capsule, device composition or method of any one of the above embodiments, wherein the FBR-mitigating compound is selected from the group consisting of Compounds 100, 101, 102, 114, 122 and 123 shown in Table 4 above, or a pharmaceutically acceptable salt of the selected compound.

59. The implantable device, hydrogel capsule, device composition or method of any one of the above embodiments, wherein the FBR-mitigating compound is Compound 100 or a pharmaceutically acceptable salt thereof.

60. The implantable device, hydrogel capsule, device composition or method of any one of the above embodiments, wherein the FBR-mitigating compound is Compound 101 or a pharmaceutically acceptable salt thereof.

61. The implantable device, hydrogel capsule, device composition or method of any one of the above embodiments, wherein the FBR-mitigating compound is Compound 114 or a pharmaceutically acceptable salt thereof.

62. The implantable device, hydrogel capsule, device composition or method of any one of the above embodiments, wherein the FBR-mitigating compound is Compound 122 or a pharmaceutically acceptable salt thereof.

63. The implantable device or hydrogel capsule, device composition or method of any one of the above embodiments, wherein the FBR-mitigating compound is Compound 123 or a pharmaceutically acceptable salt thereof.

64. A genetically modified cell comprising an exogenous nucleotide coding sequence shown in FIG. 1B, 1D, 1E, 1G, 2C, 2E, 3C, 3E or 7B.

65. The genetically modified cell of embodiment 64, which is derived from ARPE-19 cells.

### EXAMPLES

In order that the disclosure described herein may be more fully understood, the following examples are set forth. The examples described in this application are offered to illustrate the implantable devices, and compositions and methods provided herein and are not to be construed in any way as limiting their scope.

### Example 1: Culturing of Exemplary Genetically-Modified ARPE-19 Cells for Encapsulation

Genetically modified ARPE-19 cells expressing one or more immunomodulatory proteins described herein may be cultured to produce a composition of cells suitable for encapsulation in two compartment hydrogel capsules. The genetically-modified cells are grown in complete growth medium (DMEM:F12 with 10% FBS) in 150 cm2 cell culture flasks or CellSTACK® Culture Chambers (Corning Inc., Corning, NY). To passage cells, the medium in the culture flask are aspirated, and the cell layer is briefly rinsed with phosphate buffered saline (pH 7.4, 137 mM NaCl, 2.7 mM KCl, 8 mM Na₂HPO₄, and 2 mM KH₂PO₄, Gibco). 5-10 mL of 0.05% (w/v) trypsin/0.53 mM EDTA solution (“TrypsinEDTA”) is added to the flask, and the cells are observed under an inverted microscope until the cell layer is dispersed, usually between 3-5 minutes. To avoid clumping, cells are handled with care and hitting or shaking the flask during the dispersion period is minimized. If the cells do not detach, the flasks are placed at 37° ° C. to facilitate dispersal. Once the cells disperse, 10 mL complete growth medium is added and the cells are aspirated by gentle pipetting. The cell suspension is transferred to a centrifuge tube and spun down at approximately 125×g for 5-10 minutes to remove TrypsinEDTA. The supernatant is discarded, and the cells are resuspended in fresh growth medium. Appropriate aliquots of cell suspension are added to new culture vessels, which are incubated at 37° C. The medium is renewed weekly.

### Example 2: Preparation of Exemplary Modified Polymers

Chemically-modified Polymer. A polymeric material may be chemically modified with a compound of Formula (I) (or pharmaceutically acceptable salt thereof) prior to formation of a device described herein (e.g., a hydrogel capsule). For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest (e.g., Compound 101 shown in Table 4) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a solution of low molecular weight alginate, chemically modified with a medium conjugation density of Compound 101 (CM-LMW-Alg-101-Medium polymer), the dissolved unmodified low molecular weight alginate (approximate MW<75 kDa, G:M ratio ≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a solution of low molecular weight alginate, chemically modified with a high conjugation density of Compound 101 (CM-LMW-Alg-101-High polymer), the dissolved unmodified low-molecular weight alginate (approximate MW<75 kDa, G:M ratio ≥15) is treated with 2-chloro-4,6-dimethoxy-1,3,5-triazine (5.1 mmol/g alginate) and N-methylmorpholine (10.2 mmol/g alginate) and Compound 101 (10.5 mmol/g alginate).

The reaction is warmed to 55ºC for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

CBP-Alginates. A polymeric material may be covalently modified with a cell-binding peptide prior to formation of a device described herein (e.g., a hydrogel capsule described herein) using methods known in the art, see, e.g., Jeon O, et al., Tissue Eng Part A. 16:2915-2925 (2010) and Rowley, J. A. et al., Biomaterials 20:45-53 (1999).

For example, in the case of alginate, an alginate solution (1%, w/v) is prepared with 50 mM of 2-(N-morpholino)-ethanesulfonic acid hydrate buffer solution containing 0.5M NaCl at pH 6.5, and sequentially mixed with N-hydroxysuccinimide and 1-ethyl-3-[3-(dimethylamino)propyl] carbodiimide (EDC). The molar ratio of N-hydroxysuccinimide to EDC is 0.5:1.0. The peptide of interest is added to the alginate solution. The amounts of peptide and coupling reagent added depends on the desired concentration of the peptide bound to the alginate, e.g., peptide conjugation density. By increasing the amount of peptide and coupling reagent, higher conjugation density can be obtained. After reacting for 24 h, the reaction is purified by dialysis against ultrapure deionized water (diH2O) (MWCO 3500) for 3 days, treated with activated charcoal for 30 min, filtered (0.22 mm filter), and concentrated to the desired viscosity.

The conjugation density of a peptide-modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

### Example 3: Preparation of Exemplary Alginate Solutions for Making Hydrogel Capsules

70:30 mixture of chemically-modified and unmodified alginate. A low molecular weight alginate (PRONOVA™ VLVG alginate, NovaMatrix, Sandvika, Norway, cat. #4200506, approximate molecular weight <75 kDa; G:M ratio ≥1.5) is chemically modified with Compound 101 to produce chemically modified low molecular weight alginate (CM-LMW-Alg-101) solution with a viscosity of 25 cp to 35 cP and a conjugation density of 5.1% to 8% N, as determined by combustion analysis for percent nitrogen. A solution of high molecular weight unmodified alginate (U-HMW-Alg) is prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution is blended with the U-HMW-Alg solution at a volume ratio of 70% CM-LMW-Alg to 30% U-HMW-Alg (referred to herein as a 70:30 CM-Alg:UM-Alg solution).

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Alginate Solution Comprising Cell Binding Sites. A solution of SLG20 alginate is modified with a peptide consisting of GRGDSP as described above and concentrated to a viscosity of about 100 cP. The amount of the peptide and coupling reagent used are selected to achieve a target peptide conjugation density of about 0.2 to 0.3, as measured by combustion analysis.

### Example 4: Preparation of Exemplary Immunosuppressant Particles Suspended in Alginate Solutions Using Indirect Sonication

An amount of a solid, amorphous form of the desired immunosuppressant (e.g., a glucocorticoid) is added to an alginate solution (e.g., GRGDSP-medium molecular weight alginate in saline, viscosity of about 100 cP) in a conical tube (e.g., 15 mL to 50 mL) in an amount sufficient to form a suspension with a desired concentration of the solid immunosuppressant in the alginate solution (e.g., 1 mg to 10 mg compound per mL alginate solution). The tube is placed in a 5.7 liter sonication bath (Ultrasonic Cleaner, VWR Catalog No. 97043-940, Input: 117V˜60 Hz, Operating frequency: 35 kHz) at room temperature for 3 minutes to 10 minutes, removed and vortexed for one minute at 3,000 RPM (Thermo Scientific™ LP Vortex Mixer). The sonication and vortex steps are repeated until no visible powder was observed, indicating that a fine, substantially homogenous suspension has been generated. The suspension is kept at 4° C. until use and is used within 6 hours.

### Example 5: Formation of Exemplary Two-Compartment Hydrogel Capsules

Genetically modified cells, and optionally immunosuppressant particles, are encapsulated in two-compartment hydrogel capsules according to the protocol described below.

Prior to fabricating hydrogel capsules, buffers and alginate solutions are sterilized by filtration through a 0.2-μm filter using aseptic processes.

Immediately before encapsulation, a desired volume of a composition comprising the cells (e.g., from a culture of the cells as described in Example 1) is centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, PH≈7.4, ≈290 mOsm). After washing, the cells ae centrifuged again and all of the supernatant is aspirated. The cell pellet is resuspended in the GRGDSP-modified alginate solution described in Example 3 or in Example 4 at a desired cell density (e.g., about 50 to 150 million suspended single cells per ml alginate solution).

To prepare two-compartment hydrogel millicapsules of about 1.5 mm diameter an electrostatic droplet generator is set up as follows: an ES series 0-100-kV, 20-watt high-voltage power generator (EQ series, Matsusada, NC, USA) is connected to the top and bottom of a coaxial needle. For capsules without an immunosuppressant, a suitable needle has an inner lumen of 22G, outer lumen of 18G, Rame-Hart Instrument Co., Succasunna, NJ, USA. To prepare capsules that co-encapsulate immunosuppressant particles in the inner compartment, the inner lumen of the coaxial needle may need to have a larger diameter to avoid needle clogging by the immunosuppressant particles, e.g., a useful coaxial needle has an inner lumen of 21G and an outer lumen of 17G, Ramé-Hart Instrument Co., Succasunna, NJ, USA).

The inner lumen is attached to a first 5-ml Luer-lock syringe (BD, NJ, USA), which is connected to a syringe pump (Pump 11 Pico Plus, Harvard Apparatus, Holliston, MA, USA) that is oriented vertically. The outer lumen is connected via a luer coupling to a second 5-ml Luer-lock syringe which is connected to a second syringe pump (Pump 11 Pico Plus) that is oriented horizontally. A first alginate solution containing the genetically modified cells (as single cells) suspended in a GRGDSP-modified alginate solution is placed in the first syringe and a cell-free alginate solution comprising a mixture of a chemically-modified alginate and unmodified alginate is placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump are 12.06 mm diameter and the flow rates of each pump are adjusted to achieve a flow rate ratio of 1:1 for the two alginate solutions. Thus, with the total flow rate set at 10 ml/h, the flow rate for each alginate solution was about 5 mL/h. Control (empty) capsules are prepared in the same manner except that the alginate solution used for the inner compartment is a cell-free solution.

After extrusion of the desired volumes of alginate solutions, the alginate droplets are crosslinked for five minutes in a cross-linking solution which contained 25 mM HEPES buffer, 20 mM BaCl₂, 0.2M mannitol and 0.01% of poloxamer 188. Capsules that fall to the bottom of the crosslinking vessel are collected by pipetting into a conical tube. After the capsules settle in the tube, the crosslinking buffer is removed, and capsules are washed four times in HEPES buffer, two times in 0.9% saline, and two times in culture media and stored in an incubator at 37ºC.

### Example 6: Evaluating Diffusion of Glucocorticoid from Capsules Containing Encapsulated Glucocorticoid Particles

A glucocorticoid particle suspension is prepared as described in Example 4 using amounts of the test glucocorticoid (solid, amorphous form) and alginate solution to achieve a concentration of 2 mg of the glucocorticoid solid/mL alginate solution. Two-compartment hydrogel capsules containing the suspension in the inner compartment are prepared as in Example 5, except that cells are not typically included. Multiple capsules (e.g., five) are placed in each of a desired number of replicate wells (e.g., four) in a multiwell tissue culture plate (e.g., Falcon® 12-well Clear Flat Bottom Not Treated Multiwell Cell Culture Plate). Each of the replicate wells contains 1 mL of a cell culture media (e.g., DMEMF12, Gibco Cat. No. 113 30-032 with rFBS, Gibco Cat. No. 26140-095). The media in each well is replaced with fresh 1 mL of the media at day 1, day 4 and day 7. Pictures of each well are taken using a BZX Fluorescence Microscope at day 1, day 4, day 7 to visually assess the capsules for the presence of encapsulated glucocorticoid particles.

The quantity of glucocorticoid diffusing from the capsules into the media over time is estimated by removing an aliquot (e.g., 20 μL) of the media supernatant from each replicate well on multiple days (e.g., day 1, day 4 and day 7) and diluting the removed aliquot 10-fold with Methanol (e.g., 180 μL). The sample is vortexed briefly and centrifuged at 12,500×g for 10 minutes. The supernatant is removed and transferred to an LCMS vial for analysis. The samples are analyzed using a Thermo Fisher Vanquish UPLC interfaced to a Thermo Fisher Q Exactive mass spectrometer. The chromatographic separation is performed on a 50 mm×2.1 mm Waters BEH C18 chromatography column with 1.7 μm particle size. Mobile phases A and B are 0.1-% aqueous formic acid and 0.1% formic acid in acetonitrile, respectively. The column temperature is held at 65° C. and the mobile phase is flowed at 500 μL/min. Injection volume is 5 μL. The separation is accomplished with a gradient started at 10% mobile phase B and increasing to 99% over 2.5 minutes. The column is held at 99% B for 1 minute before returning to initial conditions. The initial conditions (10% B) are held for 1.5 minutes before the next LC injection. The Q Exactive mass spectrometer is configured with an electrospray ionization source operating in positive ion mode. Data are acquired using a full scan method scanning from 400-1250 m/z at a resolution of 70,000. Data are analyzed by extracting a 10 ppm window centered on the +H ions for each glucocorticoid of interest. The Retention Time for TAH is 2.41 minutes and m/z is 533.2909.

A suspension of particles of a test glucocorticoid prepared as in Example 4 is likely to provide the desired extended release of the glucocorticoid from the capsules if less than 10% of the encapsulated amount of the glucocorticoid has been released at day 7, and preferably less than <5%, <2%, or <1% has been released at day 7.

### Example 7. Continuous Delivery of IL-10 by Shielded, Encapsulated Cells Modulates Immune Cell Function and Prevents Liver Damage in Animal Model of Autoimmune Hepatitis

Autoimmune hepatitis (AIH) affects an estimated 70,000 individuals in the U.S. every year. The pathology of this disease results from a breakdown in immune tolerance leading to production of pro-inflammatory cytokines by autoreactive T-cells and subsequent hepatocyte destruction. The current standard of care clinical therapy for AIH consists of a combination of corticosteroids and azathioprine.

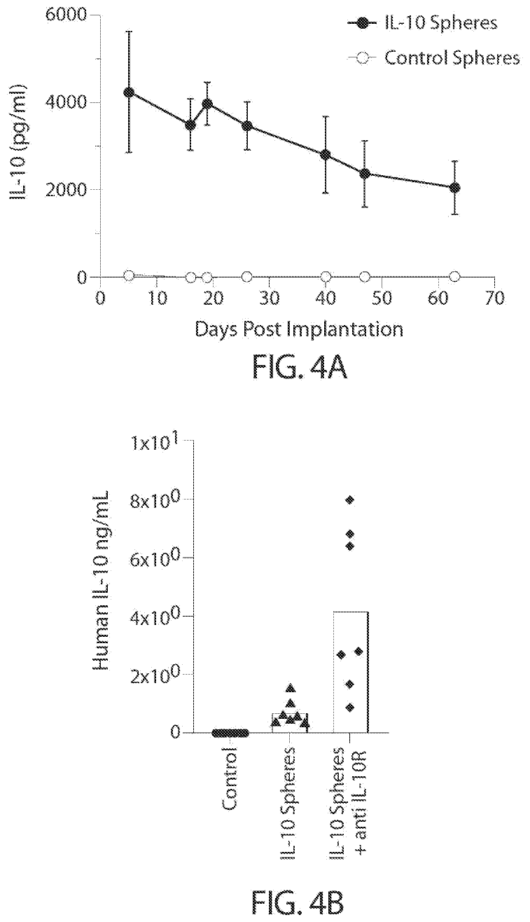

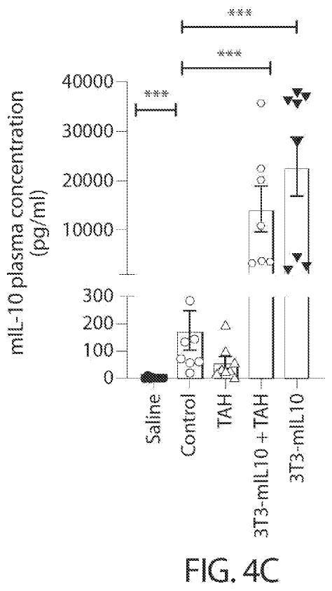

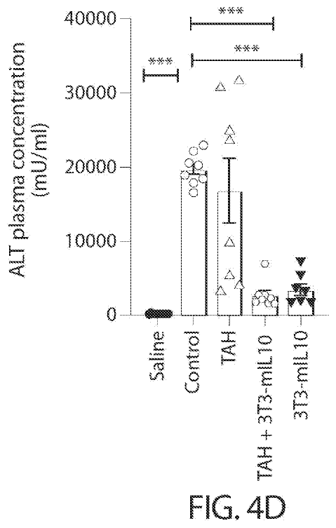

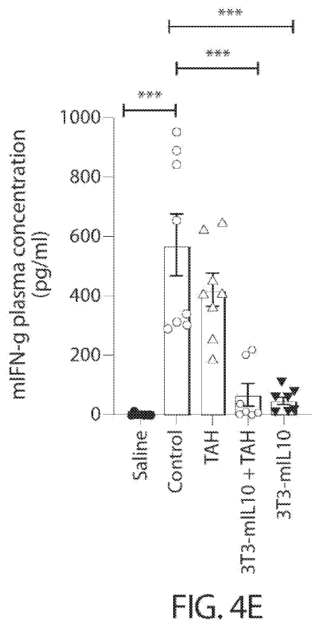

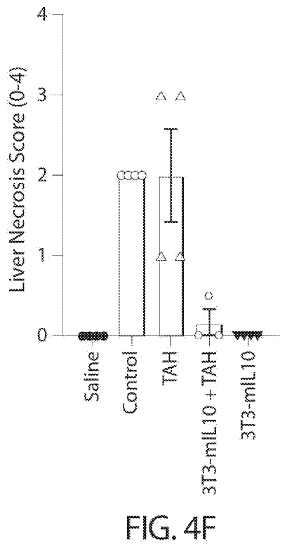

ARPE-19 cells genetically modified to express and secrete active human IL-10 were encapsulated in two-compartment hydrogel capsules as described in Example 5 (without immunosuppressant) and shown to produce functional IL-10 (data not shown). Capsules were placed intraperitoneally in mice, and sustained delivery of IL-10 was observed for greater than 2 months, as shown in FIG. 4A. Treatment of mice with an IL-10 receptor blocking antibody, led to increased circulating levels of human IL-10 to about 4 ng/ml. These data demonstrate that human IL-10 produced by the implanted capsules was functional and able to engage its cognate receptors in vivo. This result indicates that the implanted hydrogel capsules were able to provide sustained delivery of functional human IL-10 for several weeks since human IL-10 in plasma has a half-life of only about 2 hours. The delivered IL-10 resulted in in vivo differentiation of peritoneal macrophages towards the immunomodulatory M² phenotype marked by increased expression of CD206 (data not shown).

Another experiment investigated the ability of sustained delivery of IL-10, TAH or the combination of IL-10 and TAH by exemplary devices of the disclosure in the concanavalin-A model of AIH, a widely used pre-clinical murine model for immune-mediated inflammatory liver diseases such as AIH. C57Bl/6 mice were implanted with control two-compartment hydrogel capsules (no cells or immunosuppressant) or two-compartment hydrogel capsules with one of the following configurations: (i) TAH particles encapsulated in inner compartment, but no cells (TAH), (ii) 3T3 cells genetically modified to secrete mIL-10 and TAH particles co-encapsulated in the inner compartment and (iii) 3T3 cells genetically modified to secrete mIL-10, but no TAH. The mice were then injected IV with either saline or with concanavalin A (ConA) at 20 mg/kg. At 18 hours after the IV injection, levels of mIL-10, ALT, and IFN-gamma were measured in each mice group. As shown in FIG. 4C, the co-encapsulation of TAH particles with the genetically modified cells did not significantly impact plasma levels of mIL-10 produced by the implanted capsules. A significant reduction in plasma levels of pro-inflammatory IFN-gamma (FIG. 4D) and the liver enzyme ALT (FIG. 4E) was observed in mice treated with hydrogel capsules that delivered IL-10 or both IL-10 and TAH, but not in mice treated with the TAH-only capsules. Liver pathological changes were observed by histology with H&E staining and scored for necrosis by a blinded pathologist; as shown in FIG. 4F, continuous intraperitoneal delivery of IL-10, alone or in combination with sustained intraperitoneal release of TAH, protected the mice from ConA-induced acute liver injury.

### Example 8. Secretion of Human IL-22 by Encapsulated Cells Promotes IL-10 Production from Colonic Epithelial Cells

Balb-3T3 cells and ARPE-19 cells were genetically modified to express and secrete murine IL-22 and human IL-22, respectively. The ability of the genetically modified ARPE-19 cells to produce active hIL-22 in vitro was assessed by the induction of IL-10 production by Colo205 cells, which are derived from human colonic epithelium.

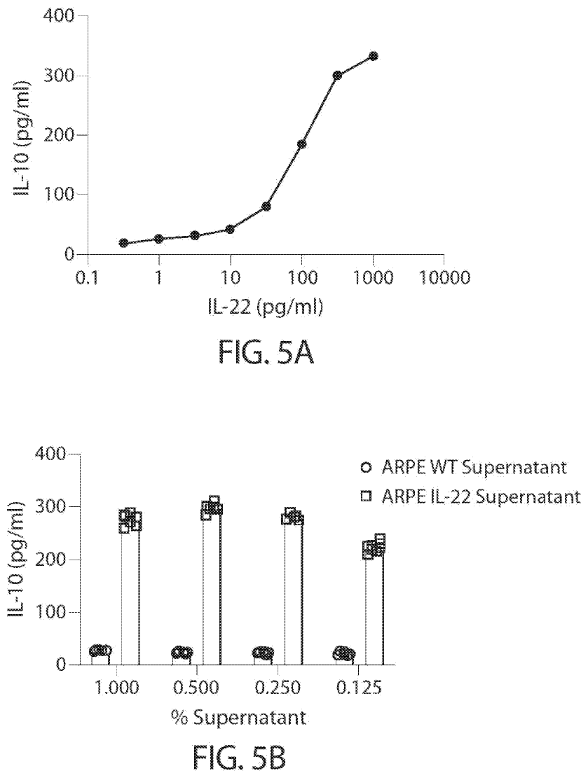

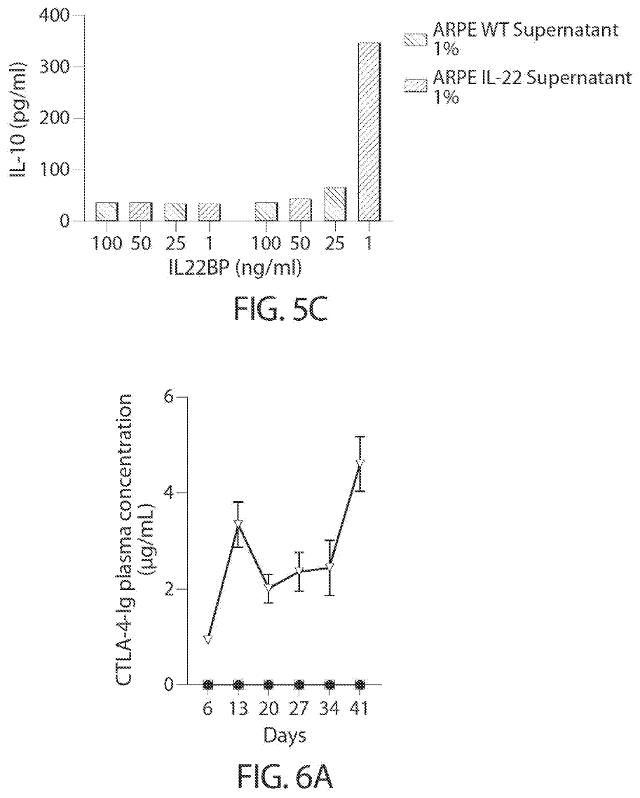

Generation of IL-22 response curve. Col205 cells were seeded at a density of 2×10⁵ cells/well in a 96 well plate and starved in serum free media overnight. The serum free media was removed from the wells and the cells were treated with rhIL-22 (peprotech Cat #200-22) diluted in RPMI/10% FBS at half log dilutions to generate final concentrations of 1000, 316.46, 100.14, 31.69, 10.02, 3.17, 1 and 0.32 pg/ml. The plate was incubated overnight at 37 C, 5% CO2 for ˜18 h. The supernatant was transferred to a new 96 well plate and centrifuged. IL-10 levels in the supernatant were measured by ELISA (abcam, ab 185986), and the results are shown in FIG. 5A.

Biological activity of hIL-22 produced by genetically modified ARPE-19 cells. Col205 cells were seeded at a density of 2×10⁵ cells/well in a 96 well plate and starved in serum free media overnight. The serum free media was removed and the cells were treated with filter sterilized supernatant from hIL22 producing ARPE-19 cells (ARPE-19 IL-22) or unmodified ARPE-19 cells (ARPE-19 WT) diluted in RPMI/10% FBS at final concentrations of 1%, 0.5%, 0.25% and 0.125%. The plate was incubated overnight at 37 C, 5% CO2 for ˜18 h. The supernatant was transferred to a new 96 well plate and centrifuged. IL-10 levels in the supernatant were measured by ELISA (abcam, ab 185986), and the results are shown in FIG. 5B.

Inhibition of IL22 activity in Col205 cells. Col205 cells were seeded at a density of 2×10⁵ cells/well in a 96 well plate and starved in serum free media overnight. The serum free media was removed and the cells were treated with 190 ul of recombinant human IL-22 binding protein (rhIL22BP) diluted in RPMI/10% FBS at concentrations of 100, 50, 25 and 1 ng/ml. Filter sterilized supernatant from hIL22-producing ARPE-19 cells (ARPE IL-22) or unmodified ARPE-19 cells (ARPE WT) was diluted 1:5 in RPMI/10% FBS and 10 ul was added to the wells for a final concentration of 1%. The plate was incubated overnight at 37 C, 5% CO2 for ˜18 h. The supernatant was transferred to a new 96 well plate and centrifuged. IL-10 levels in the supernatant were measured by ELISA (abcam, ab 185986) and the results are shown in FIG. 5C.

The results of these experiments using colonic epithelial cells demonstrate that hIL-22 produced by genetically modified cells derived from the ARPE-19 cell line was functionally active due to its ability to induce IL-10 production by the colonic epithelial cells, and this activity was specific for rhIL-22 since IL-10 production was blocked in the presence of rhIL22BP, an endogenous and specific IL-22 inhibitor.

### Example 9. Continuous Delivery of a Soluble CTLA-4 Protein and an Immunosuppressant Prevent Induction of GvHD in a Xenogeneic Transplant Model

ARPE-19 cells were genetically modified to express and secrete a CTLA-4-Ig protein with the amino acid sequence shown in FIG. 3B. CTLA-4 has been shown to block the activation of T cells when provided in combination with an immunosuppressant. TAH particles were co-encapsulated with either unmodified ARPE-19 cells (ARPE-19 WT) or the CTLA-4-Ig expressing ARPE-19 cells in the inner compartment of two-compartment hydrogel capsules. CTLA-4-Ig was secreted by the genetically modified cells, both in vitro, and when co-encapsulated with TAH particles.

Immunodeficient NSG mice were implanted intraperitoneally with CTLA+TAH capsules, ARPE-19 WT+TAH capsules or without any capsules. NSG injected with peripheral blood mononuclear cells (PBMCs) obtained from a healthy human donor. Weekly blood samples were obtained from each group of mice and plasma levels of CTLA-4-Ig in the samples were measured by ELISA [symbol] Cells in peripheral blood were preincubated with rat anti-mouse FcγRIImAb (clone 2.4G2; BD Biosciences) to block non-specific binding to murine FcRs [symbol] Fluorescently conjugated antibodies specific to human and mouse ly5 and human CD45 were then added to the samples and incubated for 30 min at 4ºC. Stained samples were washed, blood was treated with eBioscience™ 1-step Fix/Lyse Solution. Fixed samples were washed and cells were analyzed using CytoFLEX Flow Cytometer. Data analysis was performed with FlowJo software.

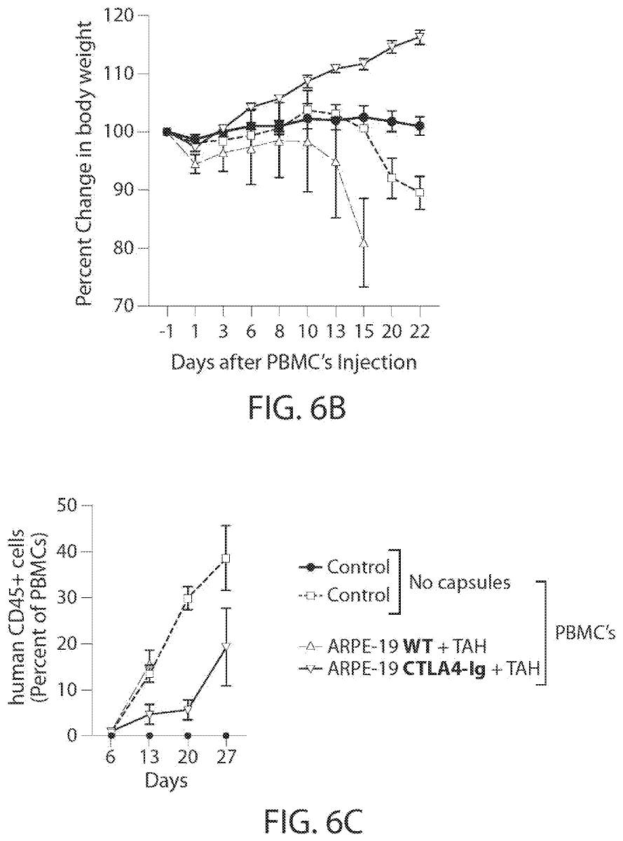

The implanted capsules continuously produced the CTLA-4-Ig protein for over 4 weeks, with plasma levels reaching 3 microgram/mL (FIG. 6A). The secreted CTLA-4-Ig protein efficiently prevented the induction of GvHD-like diseases as monitored by evaluating changes in the body weights of recipient mice. There was no significant change body weight of control mice which neither received any PBMCs nor were implanted with any spheres. In contrast mice injected with PBMCs exhibited rapid body weight loss in all groups except in the group which were implanted with spheres containing ARPE-19 cells secreting CTLA4-Ig and TAH (FIG. 6B). The continuous delivery of CTLA-4-Ig and TAH by the implanted capsules efficiently delayed the engraftment of hCD45+ cells, as measured by their abundance in weekly bleeds over one month (FIG. 6C). These data demonstrate that continuous production of CTLA-Ig along with an immunosuppressant TAH was effective in preventing the engraftment and proliferation of T cells that drive the pathogenesis in this model of GvHD.

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. An implantable device comprising a first plurality of mammalian cells genetically modified to express and secrete one or more immunomodulatory proteins, wherein the device is configured to exhibit the following properties when implanted into a subject:
(a) the subject's immune cells do not contact the genetically modified cells;
(b) the genetically modified cells do not exit the device; and
(c) deliver each immunomodulatory protein to the subject in an amount and for a time period effective to induce an anti-inflammatory immune response in the subject;
wherein the device comprises at least one of the following features:
(i) the device comprises a second plurality of mammalian cells genetically modified to express and secrete at least one immunomodulatory protein that is different than each immunomodulatory protein secreted by the first plurality of cells;
(ii) an extended release formulation of an immunosuppressant;
(iii) at least one of the immunomodulatory proteins secreted by the first plurality of genetically modified cells comprises a heterologous secretory signal peptide sequence;
(iv) a compound or polymer disposed on the exterior surface of the device that mitigates the foreign body response (FBR) to the device;
(v) the surface of the device does not contain alginate; and
(vi) the first plurality of genetically modified mammalian cells are derived from ARPE-19 cells.

2. The implantable device of claim 1, wherein the time period is at least any of 30 days, 60 days, 90 days 120 days, 180 days or longer.

3. The implantable device of claim 1, wherein the heterologous signal peptide sequence consists essentially of MGWRAAGALLLALLLHGRLLA (SEQ ID NO:21).

4. The implantable device of claim 1, wherein the anti-inflammatory immune response comprises one or both of (x) increased expression of an anti-inflammatory cytokine in the subject's plasma and (y) reduced expression of a pro-inflammatory cytokine in the subject's plasma,
optionally wherein the anti-inflammatory cytokine is IL-10 and the pro-inflammatory cytokine is tissue necrosis factor alpha (TNF-alpha) or interferon gamma (IFN-γ).

5. The implantable device of claim 1, which comprises one or more of feature (i), feature (ii) and feature (iii).

6. The implantable device of claim 1, which comprises feature (iv).

7. The implantable device claim 1, which comprises feature (v).

8. The implantable device of claim 1, which comprises feature (vi).

9. The implantable device of claim 1, wherein the cells in the first plurality of genetically modified cells comprise an exogenous nucleotide sequence encoding IL-10 or IL-22.

10. The implantable device of claim 9, wherein the genetically modified cells are derived from ARPE-19 cells.

11. The implantable device of claim 1, which comprises an extended release formulation of an immunosuppressant.

12. The implantable device of claim 1, wherein the immunosuppressant is cyclosporine, rapamycin, or a glucocorticoid.

13. The implantable device of claim 12, wherein the glucocorticoid is a compound of Formula (IV): 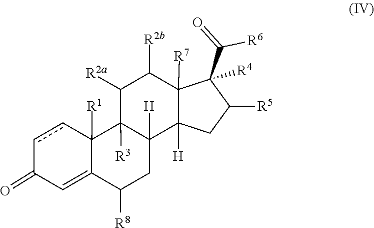
or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

14. The implantable device of claim 12, wherein the glucocorticoid is selected from the group consisting of triamcinolone hexacetonide, triamcinolone acetonide, fluticasone furoate, fluticasone propionate, mometasone furoate, and beclomethasone diproprionate.

15. The implantable device of claim 1, wherein the immunosuppressant is TAH.

16. The implantable device of claim 1, which comprises feature (iv) and feature (v).

17. The implantable device of claim 1, wherein each plurality of genetically modified cells is contained in a cell-containing compartment surrounded by a barrier compartment.

18. The implantable device of claim 1, wherein the barrier compartment comprises a hydrogel-forming polymer, e.g., an alginate.

19. The implantable device of claim 17, wherein the cell-containing compartment comprises a hydrogel-forming polymer, e.g., an alginate, a GRGDSP-modified alginate.

20. The implantable device of claim 1, which comprises two or more cell-containing compartments.

21. The implantable device of claim 1, wherein the FBR-mitigating compound is a compound of Formula (I): 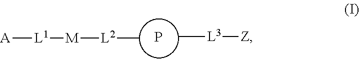
or a pharmaceutically acceptable salt thereof, wherein:
A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)—, —OS(O)—, —N(R^{C})S(O)—, —S(O)ₓN(R^{C})—, —P(R^{F})_{y}—, —Si(OR^{A})₂-, —Si(R^{G})(OR^{A}), —B(OR^{A})—, or a metal, each of which is optionally linked to an attachment group (e.g., an attachment group described herein) and is optionally substituted by one or more R¹;
each of L¹ and L³ is independently a bond, alkyl, or heteroalkyl, wherein each alkyl and heteroalkyl is optionally substituted by one or more R²;
L² is a bond;
M is absent, alkyl, heteroalkyl, cycloalkyl, heterocyclyl, aryl, or heteroaryl, each of which is optionally substituted by one or more R³;
P is absent, cycloalkyl, heterocyclyl, or heteroaryl, each of which is optionally substituted by one or more R⁴;
Z is hydrogen, alkyl, alkenyl, alkynyl, heteroalkyl, OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;
each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;
or R^{C} and R^{D}, taken together with the nitrogen atom to which they are attached, form a ring (e.g., a 5-7 membered ring), optionally substituted with one or more R⁶;
each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;
each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;
each R⁷ is independently alkyl, alkenyl, alkynyl, heteroalkyl, halogen, cyano, oxo, hydroxyl, cycloalkyl, or heterocyclyl;
x is 1 or 2; and
y is 2, 3, or 4.

22. The implantable device of claim 21, wherein the FBR-mitigating compound is selected from the compounds shown in Table 4, or a pharmaceutically acceptable salt thereof.

23. The implantable device of claim 22, wherein the FBR-mitigating compound is: 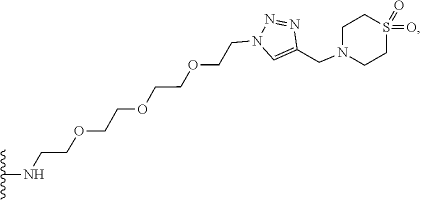 [SMILES: *NCCOCCOCCOCCn1cc(CN2CCS(=O)(=O)CC2)nn1]
or a pharmaceutically acceptable salt thereof.

24. The implantable device of claim 22, wherein the FBR-mitigating compound is 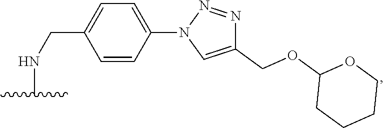 [SMILES: *NCc1ccc(-n2cc(COC3CCCCO3)nn2)cc1]
or a pharmaceutically acceptable salt thereof.

25. A hydrogel capsule comprising:
(a) a cell-containing compartment which comprises living cells encapsulated in a first polymer composition, wherein at least a portion of the living cells are genetically modified to continuously express and secrete a first immunomodulatory protein; and
(b) a barrier compartment surrounding the cell-containing compartment and comprising a second polymer composition which comprises an alginate covalently modified with at least one compound selected from the group consisting of Compound 100, Compound 101, Compound 110, Compound 112, Compound 113, Compound 114, Compound 122 and Compound 123 shown in Table 4 above, or a pharmaceutically acceptable salt of the compound,
wherein the hydrogel capsule has a spherical shape and has a diameter of 0.5 millimeter to 5 millimeters.

26. The hydrogel capsule of claim 25, which further comprises an extended release formulation of an immunosuppressant.

27. The hydrogel capsule of claim 26, wherein the extended release formulation is present in one or both of the cell-containing compartment or the barrier compartment.

28. The hydrogel capsule of claim 25, wherein the immunosuppressant is a glucocorticoid, cyclosporine or rapamycin.

29. The hydrogel capsule of claim 28, wherein the glucocorticoid is a compound of Formula (IV): 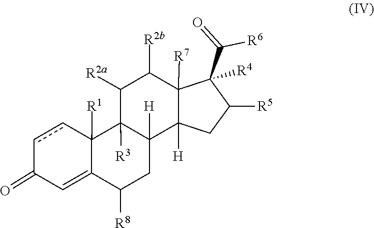
or a pharmaceutically acceptable salt, ester, hydrate, tautomer, or prodrug thereof, wherein R¹ is hydrogen, halo, or C₁-C₆ alkyl; each of R^{2a} and R^{2b} is independently hydrogen, C₁-C₆ alkyl, or —OR^{A}, wherein one of R^{2a} and R^{2b} is independently —OR^{A}; or R^{2a} and R^{2b} are taken together to form an oxo group; R³ is hydrogen, halo, or C₁-C₆ alkyl; each of R⁴ and R⁵ is independently hydrogen, halo, C₁-C₆ alkyl, or —OR^{A}; or R⁴ and R⁵ are taken together to form a ring substituted by one or more R⁹; R⁶ is hydrogen, C₁-C₆ alkyl, C₂-C₆ alkenyl, C₂-C₆ alkynyl, C₁-C₆ heteroalkyl, —OR^{A}, —N(R^{C})(R^{D}), —SR^{E}, cycloalkyl, heterocyclyl; R⁷ is hydrogen, halo, or C₁-C₆ alkyl; R⁸ is hydrogen, halo, or C₁-C₆ alkyl; R⁹ is halo, C₁-C₆ alkyl, or —OR^{A}; [symbol] is a single or double bond; and each of R^{A}, R^{B}, R^{C}, R^{D}, and R^{E} is independently hydrogen, C₁-C₆ alkyl, C(O)—C₁-C₆ alkyl, C(O)-aryl, or C(O)—C₁-C₆ heteroaryl.

30. The hydrogel capsule of claim 28, wherein the glucocorticoid is selected from the group consisting of triamcinolone hexacetonide, triamcinolone acetonide, fluticasone furoate, fluticasone propionate, mometasone furoate, and beclomethasone diproprionate.

31. The hydrogel capsule of claim 25, wherein the immunosuppressant is selected from the group consisting of:
(i) triamcinolone or an ester derivative thereof (e.g., triamcinolone hexacetonide (TAH), triamcinolone acetonide, triamcinolone benetonide, triamcinolone diacetate);
(ii) fluticasone or an ester derivative thereof (e.g., fluticasone furoate, fluticasone propionate); and
(iii) mometasone or an ester derivative thereof (e.g., mometasone furoate).

32. The hydrogel capsule of claim 31, wherein the immunosuppressant compound is TAH.

33. The hydrogel capsule of claim 25, wherein the first polymer composition comprises a hydrogel-forming polymer (e.g., an alginate, a GRGDSP-modified alginate) and the extended release formulation of TAH is prepared by a process which comprises adding a desired quantity of an amorphous powder of TAH to a desired volume of a solution comprising the hydrogel-forming polymer, sonicating the resulting mixture until a substantially homogenous suspension is formed, adding the living cells to the suspension and contacting droplets of the polymer, TAH and cell suspension with a cross-linking solution.

34. The hydrogel capsule of claim 33, wherein the quantity of TAH powder and the volume of the polymer solution are selected to achieve a mixture of 2.5 mg to 5.0 mg powder per mL polymer solution.

35. The hydrogel capsule of claim 25, wherein the barrier compartment comprises an alginate covalently modified with 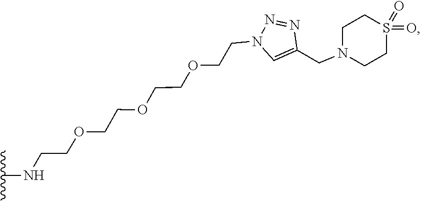 [SMILES: *NCCOCCOCCOCCn1cc(CN2CCS(=O)(=O)CC2)nn1]
or a pharmaceutically acceptable salt thereof.

36. The hydrogel capsule of claim 25, wherein the barrier compartment has an average thickness of about 10 to about 300 microns, about 20 to about 150 microns, or about 40 to about 75 microns.

37. The hydrogel capsule of claim 25, wherein the barrier compartment further comprises an unmodified alginate.

38. The hydrogel capsule of claim 25, wherein at least a portion of the living cells are genetically modified to continuously express and secrete a second immunomodulatory protein, wherein the first and second immunomodulatory proteins are expressed and secreted by the same cells or by different cells.

39. The hydrogel capsule of claim 25, wherein all of the genetically modified cells in the capsule are derived from ARPE-19 cells.

40. The hydrogel capsule of claim 25, wherein the first immunomodulatory protein is an IL-10 protein, optionally wherein the IL-10 protein is encoded by an exogenous nucleotide sequence shown in FIG. 1B, 1D, 1E or 1F.

41. The hydrogel capsule of claim 25, wherein the first immunomodulatory protein is an IL-22 protein, optionally wherein the IL-22 protein is encoded by an exogenous nucleotide sequence shown in FIG. 2C or 2E.

42. The hydrogel capsule of claim 25, wherein the first immunomodulatory protein is a soluble CTLA-4 protein, optionally wherein the CTLA-4 protein is encoded by an exogenous nucleotide sequence shown in FIG. 3C or 3E.

43. A device composition comprising a preparation of hydrogel capsules and a pharmaceutically acceptable excipient, wherein each hydrogel capsule in the preparation is a hydrogel capsule as defined in claim 25.

44. The device composition of claim 43, which has a volume of less than 10 milliliters, optionally less than 8 ml, or less than 5 ml.

45. A method of treating a subject in need of therapy with an immunomodulatory protein, comprising administering to the subject the device of any one of claims 1 to 22, the hydrogel capsule of any one of claims 25 to 42 or the device composition of any one of claim 43 or 44.

46. The method of claim 45, wherein the device, capsule or device composition is administered by implantation into the peritoneal cavity of the subject.

47. A genetically modified cell comprising an exogenous nucleotide coding sequence shown in any one of FIG. 1B, 1D, 1E, 1F, 2C, 2E, 3C, 3E or 7B.

48. The genetically modified cell of claim 47, which is derived from ARPE-19 cells.
